(19) United States
(12) Patent Application Publication
(10) Pub. No.: US 2024/0228638 A1
(43) Pub. Date: Jul. 11, 2024

(54) Method for Treating Chronic Obstructive Pulmonary Disease With an ST2 Antagonist

(71) Applicant: Genentech, Inc., South San Francisco, CA (US)

(72) Inventors: Michele Anne Grimbaldeston, Menlo Park, CA (US); Divya Mohan, South San Francisco, CA (US); Ahmed Yousuf, Leicester (GB); Christopher Brightling, Leicester (GB); Dorothy Sze-Wing Cheung, Palo Alto, CA (US); David Fong Choy, South San Fransciso, CA (US)

(73) Assignee: Genentech, Inc., South San Francisco, CA (US)

(21) Appl. No.: 18/528,122

(22) Filed: Dec. 4, 2023

Related U.S. Application Data
(63) Continuation of application No. PCT/US2022/032979, filed on Jun. 10, 2022.
(60) Provisional application No. 63/209,624, filed on Jun. 11, 2021.

(51) Int. Cl. C07K 16/28; A61K 39/00; A61P 11/00

(52) U.S. Cl. CPC C07K 16/2866; A61P 11/00; A61K 2039/505; A61K 2039/545

(57) ABSTRACT

The application describes a method of treating chronic obstructive pulmonary disease (COPD) in a patient comprising administering 476 mg of an ST2 antagonist to the patient on Day 1 of a treatment period. The application also describes methods of treating or preventing frequency of moderate to severe exacerbations in a patient having COPD comprising administering an effective amount of an ST2 antagonist to achieve a clinical improvement of at least 10%, at least 20%, at least 21%, at least 22%, at least 25%, at least 30%, at least 35%, at least 40% or at least 45% annualized exacerbation rate reduction than standard of care (SOC).

### CROSS-REFERENCE TO RELATED APPLICATIONS

This application is a continuation of International Application No. PCT/US2022/032979, filed Jun. 10, 2022, which claims the benefit of priority of U.S. Provisional Application No. 63/209,624, filed Jun. 11, 2021, which is incorporated by reference herein in its entirety for any purpose.

### SEQUENCE LISTING

The present application contains a Sequence Listing that has been submitted electronically in XML format. Said XML copy, created on Nov. 27, 2023, is named “01146-0108-04US_ST26.xml” and is 39,852 bytes in size. The information in the electronic format of the sequence listing is incorporated herein by reference in its entirety.

### FIELD OF THE INVENTION

The invention concerns methods of treating chronic obstructive pulmonary disease (COPD) in patients with an ST2 antagonist.

### BACKGROUND OF THE INVENTION

ST2 is a binding receptor for interleukin-33 (IL-33), a cytokine related to IL-1 and IL-18 and also known as NF-HEV or IL-1F11. ST2 is expressed as both a soluble non-signaling variant (soluble ST2 or sST2) and a full-length membrane-spanning form (FL ST2, ST2 or ST2L) that mediates cellular responses to IL-33. The latter form is expressed on a wide range of cell types implicated in pathologic inflammation in a number of disease settings. These include lymphocytes, particularly IL-5 and IL-13-expressing T helper cells, natural killer (NK) and natural killer-T (NKT) cells, as well as many so-called innate immune cells, such as mast cells, basophils, eosinophils, macrophages and type 2 innate lymphoid cells (ILC2) (Neill, Wong et al. 2010). IL-33 binding to ST2 on these cells leads to the recruitment of a broadly-expressed co-receptor known as the IL-1R Accessory Protein (AcP) and the activation of pro-inflammatory signaling, similar to IL-1 and IL-18. IL-33 is thus able to directly activate ST2-expressing cells or enhance their activation when in the presence of other activating stimuli. Examples of IL-33-induced cellular responses include the production of inflammatory cytokines, such as IL-5, IL-6, IL-13, TNF, IFN-γ and GM-CSF as well as the production of chemokines, such as CXCL8, CCL17 and CCL24. IL-33 has also been shown to enhance acute allergic responses by augmenting mast cell and basophil activation triggered by IgE receptor signaling or other mast cell and basophil activators. IL-33 will also enhance the recruitment, survival and adhesive properties of ST2 expressing immune cells and thus is important in provoking and sustaining cellular inflammation in local tissues.

The pro-inflammatory actions of IL-33 on innate and adaptive immune cells culminate to promote a number of pathologic processes. In the lungs, these include increased airway inflammation, mucus production, airway hyper responsiveness and fibrotic remodeling. IL-33 can also contribute to localized inflammation in the joints as well as cutaneous and articular hypernociception, by promoting the production of proinflammatory cytokines (Verri, Guerrero et al. 2008; Xu, Jiang et al. 2008). Excessive IL-33 has been linked to pathologic collagen deposition and fibrosis and also contributes to epithelial damage in the setting of inflammatory bowel disease. Through its potent effects on basophils and IgE-sensitized mast cells, IL-33 can also trigger anaphylactic shock (Pushparaj, Tay et al. 2009) and may play a contributing role in allergic disease. Many of these diseases are chronic and progressive in nature and difficult to treat and there is a need for more effective treatments.

Additional evidence linking the IL-33/ST2 pathway to human disease is provided by genetic studies, which have identified IL-33 and/or ST2 gene polymorphisms in the general population that are significantly associated with increased risk of disease or parameters of disease severity. Several large genome-wide association studies have linked genetic variation in ST2 (IL1RL1) or IL-33 with increased risk of asthma (Gudbjartsson, Bjomsdottir et al. 2009; Moffatt, Gut et al. 2010; Wu, Romieu et al. 2010) and other studies have genetically linked this pathway to increased asthma severity (Ali, Zhang et al. 2009) and bronchial hyper responsiveness (Reijmerink, Postma et al. 2008). Similar findings have genetically implicated this pathway in allergic disorders such as atopic dermatitis (Shimizu, Matsuda et al. 2005), rhinosinusitis (Sakashita, Yoshimoto et al. 2008; Castano R 2009) as well as nasal polyposis (Buysschaert, Grulois et al. 2010).

Chronic obstructive pulmonary disease (COPD) was the third leading cause of death globally in 2019, causing approximately 6% of the world's total deaths (WHO Factsheet 2020). The WHO estimates that 65 million people currently have moderate to severe COPD (WHO 2021). Development of COPD is attributed to long-term exposure to inhaled cigarette smoke or noxious particles such as smoke from biomass fuels. Although cigarette smoking has traditionally been the single most important risk factor for COPD, there is consistent evidence that non-smokers can also develop COPD (Lamprecht et al. 2011). The risk of COPD increases with age and usually occurs in patients aged older than 40 years; the prevalence is greater among men than women (Landis et al. 2014).

COPD is characterized by persistent respiratory symptoms and airflow limitation caused by airway and/or alveolar abnormalities, usually as a result of significant exposure to noxious gases or particles (GOLD 2021). The chronic airflow limitation is caused by a mixture of small airways diseases and parenchymal destruction (emphysema), which may be associated with narrowing of small airways and decreased lung elastic recoil (GOLD 2021). Clinically, the characteristic symptoms of COPD can include dyspnea, cough, and sputum production. COPD is a heterogeneous and progressive disease, with progression being strongly associated with airway-wall thickening and airflow limitation. The Global Initiative for Chronic Obstructive Lung Disease (GOLD) has introduced a classification of airflow limitation severity based on measurements of the forced expiratory volume in 1 second (FEV1) and by the ratio of FEV1 to the forced vital capacity (FVC). This assessment has been widely accepted as a key marker for disease progression (Hogg et al. 2004, Celli et al. 2008, GOLD 2021).

Another key marker of disease activity is COPD exacerbation, defined as an acute worsening of respiratory symptoms that result in additional therapy (GOLD 2021). While there exists some variability in the definition of exacerbation severity, severity is often classified as mild (increase in respiratory symptoms controllable by an increase of usual medication), moderate (requiring treatment with systemic corticosteroids and/or antibiotics), or severe (requiring hospitalization) (Solem et al., 2013, GOLD 2021). An increasing rate of COPD exacerbations is associated with decline in lung function, reduced quality of life, and death (Miravitlles et al. 2004. Halpin et al. 2012). Although the frequency of exacerbations increases with the severity of disease (Halpin et al. 2012), exacerbations affect individuals even with moderate COPD. Prior history of exacerbation, rather than airflow limitation, is the single best predictor for increased risk of future exacerbations (Hurst et al. 2010). COPD exacerbations are not only a major cause of morbidity and mortality, but also account for the greatest proportion of the total COPD healthcare costs (AbuDagga et al. 2013, Solem et al. 2013).

Current treatment options for COPD include non-pharmacologic and pharmacologic measures. Smoking cessation is a vital intervention for patients who continue to smoke and has the greatest potential impact on the natural course of COPD. Pulmonary rehabilitation similarly represents an important intervention for people with COPD, yet application remains low due to a variety of factors, including transportation, cost, and access. Pharmacologic treatment options include inhaled bronchodilators (beta-agonists and anticholinergics), inhaled and systemic corticosteroids, azithromycin, and phosphodiesterase inhibitors (GOLD 2021). Despite these treatment options, deceleration of disease progression and prevention of COPD exacerbations are still unmet needs (Patalano et al. 2014, Diette et al. 2015). Alternative and more effective therapeutic approaches that will relieve patient symptoms and alter the disease trajectory are urgently needed (Cazzola et al. 2016). Thus, there remains an urgent need for effective treatments for chronic obstructive pulmonary disease.

### SUMMARY OF THE INVENTION

The present invention provides, in some embodiments, methods of treating chronic obstructive pulmonary disease (COPD) using ST2 antagonists.

Embodiment 1. A method of treating chronic obstructive pulmonary disease (COPD) in a patient comprising administering 476 mg of an ST2 antagonist to the patient on Day 1 of a treatment period.

Embodiment 2. A method of reducing the frequency of moderate to severe exacerbations in a patient having COPD comprising administering 476 mg of an ST2 antagonist to the patient on Day 1 of a treatment period.

Embodiment 3. A method of reducing the frequency of moderate to severe exacerbations in a patient having COPD comprising administering an effective amount of an ST2 antagonist to achieve a clinical improvement of at least 10%, at least 20%, at least 21%, at least 22%, at least 25%, at least 30%, at least 35%, at least 40% or at least 45% annualized exacerbation rate reduction than standard of care (SOC).

Embodiment 4. A method of reducing the frequency of moderate to severe exacerbations in a patient having COPD comprising administering an ST2 antagonist to the patient in an amount effective to achieve a greater clinical improvement in the number of exacerbations than standard of care (SOC), said patient having a baseline blood eosinophil count <300 eosinophils/μL.

Embodiment 5. A method of reducing the frequency of moderate to severe exacerbations in a patient having COPD comprising administering an ST2 antagonist to the patient in an amount effective to achieve a greater clinical improvement in the number of exacerbations than SOC, said patient having a baseline blood eosinophil count ≤170 eosinophils/μL.

Embodiment 6. A method of reducing the frequency of moderate to severe exacerbations in a patient having COPD comprising administering an ST2 antagonist to the patient in an amount effective to achieve a greater clinical improvement in the number of exacerbations than SOC, said patient having a post-bronchodilator (post-BD) spirometry measurement of <0.7 as measured by forced expiratory volume in one second (FEV1) and/or forced vital capacity (FVC).

Embodiment 7. A method of reducing the frequency of moderate to severe exacerbations in a patient having COPD comprising administering an ST2 antagonist to the patient in an amount effective to achieve a greater clinical improvement in the number of exacerbations than SOC, said patient having a modified Medical Research Council (mMRC) dyspnea scale score ≥2 and a COPD assessment test score (CAT) of ≥10.

Embodiment 8. A method of treating or preventing COPD comprising administering an ST2 antagonist to a patient in an amount effective to achieve a greater clinical improvement than SOC as measured by patient reported outcome (PRO), wherein the PRO is an improvement of at least about 1, at least about 2, at least about 3, or at least about 4 points from baseline in a St. George's Respiratory Questionnaire for COPD patients (SGRQ-C) at 4 weeks, 12 weeks, 24 weeks, 36 weeks, or 48 weeks from the start of treatment.

Embodiment 9. A method for maintaining and/or improving lung function in a patient having COPD comprising administering an ST2 antagonist to the patient in an amount effective to achieve a greater clinical improvement in lung function than SOC, wherein clinical improvement is demonstrated by a mean difference compared to baseline of at least 0.04 L, 0.05 L, 0.06 L, 0.07 L, 0.08 L, or 0.09 L as measured by post-BD FEV1 at 4 weeks, 12 weeks, 24 weeks, 36 weeks, or 48 weeks from the start of treatment.

Embodiment 10. A method of improving baseline blood eosinophil count in a patient having COPD comprising administering an ST2 antagonist to the patient in an amount effective to reduce mean blood eosinophil count by at least about 25%, e.g., at least about 30%, at least about 35%, at least about 40%, at least about 45% compared to baseline, after about 4 weeks, 12 weeks, 24 weeks, 36 weeks, or 48 weeks following administration of a first dose of an ST2 antagonist.

Embodiment 11. A method of improving baseline blood eosinophil count in a patient having COPD comprising administering an ST2 antagonist to the patient in an amount effective to reduce mean blood eosinophil count by at least about 25%, e.g., at least about 30%, at least about 35%, at least about 40%, at least about 45% compared to baseline, after about 4 weeks following administration of a first dose of an ST2 antagonist.

Embodiment 12. A method of reducing the frequency of moderate to severe exacerbations in a patient having COPD comprising administering an ST2 antagonist to the patient in an amount effective to achieve a reduction of at least about 25%, e.g., at least about 30%, at least about 35%, at least about 40%, or at least about 45% in the number of moderate to severe exacerbations at 50 weeks and/or 52 weeks from the start of treatment, as measured by annualized exacerbation rate as compared to SOC.

Embodiment 13. A method for maintaining and/or improving lung function in a patient having COPD comprising administering an ST2 antagonist to the patient in an amount effective to achieve a greater clinical improvement in lung function than SOC, wherein clinical improvement is demonstrated by a mean difference compared to baseline of at least about 5% as measured by post-BD FEV1 at 4 weeks, 12 weeks, 24 weeks, 36 weeks, or 48 weeks from the start of treatment.

Embodiment 14. A method of treating COPD in a patient comprising administering an effective amount of an ST2 antagonist to the patient, wherein the patient is selected for treatment on the basis of the genotype of the patient determined to comprise a TT allele or CT allele at polymorphism rs10206753.

Embodiment 15. A method of reducing the frequency of moderate to severe exacerbations in a patient having COPD comprising administering an effective amount of an ST2 antagonist to the patient, wherein the patient is selected for treatment on the basis of the genotype of the patient determined to comprise a TT allele or CT allele at polymorphism rs10206753.

Embodiment 16. A method of treating COPD in a patient comprising administering an effective amount of an ST2 antagonist to the patient, wherein the patient is selected for treatment on the basis of the level of sST2 in a sample derived from the patient is determined to be at or above a reference level of sST2.

Embodiment 17. A method of reducing the frequency of moderate to severe exacerbations in a patient having COPD comprising administering an effective amount of an ST2 antagonist to the patient, wherein the patient is selected for treatment on the basis of the level of sST2 in a sample derived from the patient is determined to be at or above a reference level of sST2.

Embodiment 18. The method of embodiment 16 or embodiment 17, wherein the reference level of sST2 is at least 1 ng/mL, 5 ng/mL, 10 ng/mL, 15 ng/mL, 19 ng/mL.

Embodiment 19. A method of treating COPD in a patient comprising administering an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient.

Embodiment 20. A method of reducing the frequency of moderate to severe exacerbations in a patient having COPD comprising administering an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient.

Embodiment 21. A method of treating COPD in a patient comprising administering an effective amount of an ST2 antagonist to the patient, wherein the patient is selected for treatment on the basis of the level of baseline α-diversity in a sample derived from the patient is determined to be below a reference level of alpha-diversity index.

Embodiment 22. A method of reducing the frequency of moderate to severe exacerbations in a patient having COPD comprising administering an effective amount of an ST2 antagonist to the patient, wherein the patient is selected for treatment on the basis of the level of baseline α-diversity in a sample derived from the patient is determined to be below a reference level of alpha-diversity.

Embodiment 23. The method of embodiment 21 or embodiment 22, wherein the reference level of baseline α-diversity is an α-diversity index of about 3.4, as calculated by Shannon-Weaver method.

Embodiment 24. The method of embodiment 21 or embodiment 22, wherein the reference level of baseline α-diversity is an α-diversity index is in the range of about 0 to 5 as calculated by Shannon-Weaver method.

Embodiment 25. The method of any one of embodiments 16 to 24, wherein the sample is a blood, serum, plasma, or urine sample.

Embodiment 26. The method of any one of embodiments 16 to 24, wherein the sample is a serum sample.

Embodiment 27. The method of any one of embodiments 3 to 26, comprising administering 476 mg of the ST2 antagonist to the patient on Day 1 of a treatment period.

Embodiment 28. The method of any one of the preceding embodiments, comprising administering the ST2 antagonist every 4 weeks.

Embodiment 29. The method of any one of the preceding embodiments, comprising administering the ST2 antagonist every 2 weeks.

Embodiment 30. The method of any one of the preceding embodiments, comprising administering 476 mg of the ST2 antagonist every 4 weeks.

Embodiment 31. The method of any one of the preceding embodiments, comprising administering 476 mg of the ST2 antagonist every 2 weeks.

Embodiment 32. The method of any one of embodiments 3 to 26, 28, or 29, comprising administering 490 mg of the ST2 antagonist.

Embodiment 33. The method of any one of embodiments 3 to 26, 28, or 29, comprising administering 490 mg of the ST2 antagonist every 4 weeks.

Embodiment 34. The method of any one of embodiments 3 to 26, 28, or 29, comprising administering 490 mg of the ST2 antagonist every 2 weeks.

Embodiment 35. The method of any one of the preceding embodiments, comprising subcutaneous administration of the ST2 antagonist.

Embodiment 36. The method of any one of the preceding embodiments, wherein the patient has had two or more moderate-to-severe exacerbations within a 12-month period prior to treatment.

Embodiment 37. The method of any one of the preceding embodiments, wherein the patient has a mMRC dyspnea score ≥2.

Embodiment 38. The method of any one of the preceding embodiments, wherein the patient has post-bronchodilator FEV1≥20 and <80% of predicted normal value.

Embodiment 39. The method of any one of the preceding embodiments, wherein the patient has post-bronchodilator FEV1/FVC<0.7.

Embodiment 40. The method of any one of the preceding embodiments, which achieves a greater improvement in clinical outcome compared to standard of care (SOC).

Embodiment 41. The method of any one of the preceding embodiments, which reduces the number of moderate to severe exacerbations as measured by annualized exacerbation rate reduction (AERR) as compared to SOC at 4 weeks, 12 weeks, 24 weeks, 36 weeks, 48 weeks, 50 weeks, or 52 weeks from the start of treatment.

Embodiment 42. The method of any one of the preceding embodiments, which reduces the number of moderate to severe exacerbations as measured by AERR by at least about 25%, at least about 30%, at least about 35%, at least about 40%, or at least about 45% as compared to SOC.

Embodiment 43. The method of any one of the preceding embodiments, which increases the time to first moderate or severe COPD exacerbation as compared to SOC.

Embodiment 44. The method of any one of the preceding embodiments, which improves absolute change from baseline in health-related quality of life (HRQoL) as assessed through a St.

George's Respiratory Questionnaire for COPD patients (SGRQ-C) total score as compared to SOC at 4 weeks, 12 weeks, 24 weeks, 36 weeks, 48 weeks, 50 weeks, or 52 weeks from the start of treatment.

Embodiment 45. The method of any one of the preceding embodiments, which improves the proportion of patients with improvement of HRQoL, defined as a decrease from baseline of ≥4 points in SGRQ-C total score at 4 weeks, 12 weeks, 24 weeks, 36 weeks, 48 weeks, 50 weeks, or 52 weeks from the start of treatment.

Embodiment 46. The method of any one of the preceding embodiments, which improves absolute change from baseline post-bronchodilator in forced expiratory volume in one second (FEV1) (liters) at 4 weeks, 12 weeks, 24 weeks, 36 weeks, 48 weeks, 50 weeks, or 52 weeks from the start of treatment.

Embodiment 47. The method of any one of the preceding embodiments, which improves absolute change from baseline in Evaluating Respiratory Symptoms in COPD (ERS:COPD) total score from baseline at 4 weeks, 12 weeks, 24 weeks, 36 weeks, 48 weeks, 50 weeks, or 52 weeks from the start of treatment.

Embodiment 48. The method of any one of the preceding embodiments, which improves the annualized rate of severe COPD exacerbations at 4 weeks, 12 weeks, 24 weeks, 36 weeks, 48 weeks, 50 weeks, or 52 weeks from the start of treatment.

Embodiment 49. The method of any one of the preceding embodiments, which improves absolute change from baseline in five-repetition sit-to-stand test (5STS) time (seconds) at 4 weeks, 12 weeks, 24 weeks, 36 weeks, 48 weeks, 50 weeks, or 52 weeks from the start of treatment.

Embodiment 50. The method of any one of the preceding embodiments, which improves annualized rate of EXAcerbations of Chronic Pulmonary Disease Tool and Evaluating Respiratory Symptoms in COPD (EXACT)-defined exacerbation events from baseline at 4 weeks, 12 weeks, 24 weeks, 36 weeks, 48 weeks, 50 weeks, or 52 weeks from the start of treatment.

Embodiment 51. The method of any one of the preceding embodiments, which improves an EXACT exacerbation event.

Embodiment 52. The method of any one of the preceding embodiments, which improves at least one non-E-RS COPD domain from baseline at 4 weeks, 12 weeks, 24 weeks, 36 weeks, 48 weeks, 50 weeks, or 52 weeks from the start of treatment.

Embodiment 53. The method of embodiment 52, wherein the non-E-RS COPD domain is tiredness/weakness, sleep disturbance, or fear/worry.

Embodiment 54. The method of any one of the preceding embodiments, which improves the proportion of patients with HRQoL improvement, defined as a decrease from baseline of ≥4 points in SGRQ-C total score, at 4 weeks, 12 weeks, 24 weeks, 36 weeks, 48 weeks, 50 weeks, or 52 weeks from the start of treatment.

Embodiment 55. The method of any one of the preceding embodiments, which improves the proportion of patients with symptom improvement, defined as decrease from baseline of ≥2 points from baseline in E-RS:COPD total score, at 4 weeks, 12 weeks, 24 weeks, 36 weeks, 48 weeks, 50 weeks, or 52 weeks from the start of treatment.

Embodiment 56. The method of any one of the preceding embodiments, which results in symptom improvement in the patient, defined as decrease from baseline of ≥2 points from baseline in E-RS:COPD total score, at 4 weeks, 12 weeks, 24 weeks, 36 weeks, 48 weeks, 50 weeks, or 52 weeks from the start of treatment.

Embodiment 57. The method of any one of the preceding embodiments, which improves the E-RS:COPD cough and sputum domain from baseline at 4 weeks, 12 weeks, 24 weeks, 36 weeks, 48 weeks, 50 weeks, or 52 weeks from the start of treatment.

Embodiment 58. The method of any one of the preceding embodiments, which improves the E-RS:COPD breathlessness domain from baseline at 4 weeks, 12 weeks, 24 weeks, 36 weeks, 48 weeks, 50 weeks, or 52 weeks from the start of treatment.

Embodiment 59. The method of any one of the preceding embodiments, which improves the E-RS:COPD chest symptom domain from baseline at 4 weeks, 12 weeks, 24 weeks, 36 weeks, 48 weeks, 50 weeks, or 52 weeks from the start of treatment.

Embodiment 60. The method of any one of the preceding embodiments, which improves the absolute change from baseline in post-bronchodilator FEV1 (liters) at 4 weeks, 12 weeks, 24 weeks, 36 weeks, 48 weeks, 50 weeks, or 52 weeks from the start of treatment.

Embodiment 61. The method of any one of the preceding embodiments, which improves the annualized rate of moderate COPD exacerbations at 4 weeks, 12 weeks, 24 weeks, 36 weeks, 48 weeks, 50 weeks, or 52 weeks from the start of treatment.

Embodiment 62. The method of any one of the preceding embodiments, which improves the duration of hospital stay for severe COPD exacerbations.

Embodiment 63. The method of any one of the preceding embodiments, which reduces healthcare utilization for severe COPD exacerbations.

Embodiment 64. The method of any one of the preceding embodiments, which improves the proportion of severe COPD exacerbations requiring hospital readmission within 30 days.

Embodiment 65. The method of any one of the preceding embodiments, which improves the absolute change from baseline in residual volume/forced lung capacity ratio from baseline at 4 weeks, 12 weeks, 24 weeks, 36 weeks, 48 weeks, 50 weeks, or 52 weeks from the start of treatment.

Embodiment 66. The method of any one of the preceding embodiments, which improves the absolute change from baseline in daily step count at 4 weeks, 12 weeks, 24 weeks, 36 weeks, 48 weeks, 50 weeks, or 52 weeks from the start of treatment.

Embodiment 67. The method of any one of the preceding embodiments, which improves the absolute change from baseline in time in moderate and vigorous physical activity at 4 weeks, 12 weeks, 24 weeks, 36 weeks, 48 weeks, 50 weeks, or 52 weeks from the start of treatment.

Embodiment 68. The method of any one of the preceding embodiments, which improves the absolute change from baseline in COPD Assessment Test (CAT) score at 4 weeks, 12 weeks, 24 weeks, 36 weeks, 48 weeks, 50 weeks, or 52 weeks from the start of treatment.

Embodiment 69. The method of any one of the preceding embodiments, which improves the annualized rate of moderate and severe COPD exacerbations over a blinded treatment period.

Embodiment 70. The method of any one of the preceding embodiments, which improves health-related quality of life as measured by patient reported outcome (PRO) as compared to SOC.

Embodiment 71. The method of any one of the preceding embodiments, which improves PRO as assessed through SGRQ-C by at least about 1, at least about 2, at least about 3, or at least about 4 points from baseline at 4 weeks, 12 weeks, 24 weeks, 36 weeks, 48 weeks, 50 weeks, or 52 weeks from the start of treatment.

Embodiment 72. The method of any one of the preceding embodiments, which improves FEV1 by at least 5% from baseline at 4 weeks, 12 weeks, 24 weeks, 36 weeks, 48 weeks, 50 weeks, or 52 weeks from the start of treatment.

Embodiment 73. The method of any one of the preceding embodiments, which improves ERS:COPD total score from baseline by a decrease of at least about 2 points, at 4 weeks, 12 weeks, 24 weeks, 36 weeks, 48 weeks, 50 weeks, or 52 weeks from the start of treatment.

Embodiment 74. The method of any one of the preceding embodiments, which improves the absolute change from baseline in rescue inhaler use at 4 weeks, 12 weeks, 24 weeks, 36 weeks, 48 weeks, 50 weeks, or 52 weeks from the start of treatment.

Embodiment 75. The method of any one of the preceding embodiments, which improves the absolute change from baseline in nightly total sleep time at 4 weeks, 12 weeks, 24 weeks, 36 weeks, 48 weeks, 50 weeks, or 52 weeks from the start of treatment.

Embodiment 76. The method of any one of the preceding embodiments, wherein the ST2 antagonist is administered to the patient in combination with SOC.

Embodiment 77. The method of any one of the preceding embodiments, wherein the ST2 antagonist is administered to the patient in combination with inhaled corticosteroids (ICS).

Embodiment 78. The method of any one of the preceding embodiments, wherein the ST2 antagonist is administered to the patient in combination with ICS≥500 mcg/day fluticasone propionate dose-equivalent.

Embodiment 79. The method of any one of the preceding embodiments, wherein the ST2 antagonist is administered to the patient in combination with ICS plus long-acting betaagonist (LABA).

Embodiment 80. The method of any one of the preceding embodiments, wherein the ST2 antagonist is administered to the patient in combination with ICS≥500 mcg/day fluticasone propionate dose-equivalent plus LABA.

Embodiment 81. The method of any one of the preceding embodiments, wherein the ST2 antagonist is administered to the patient in combination with Long-acting muscarinic antagonist (LAMA) plus LABA.

Embodiment 82. The method of any one of the preceding embodiments, wherein the ST2 antagonist is administered to the patient in combination with ICS plus LAMA plus LABA.

Embodiment 83. The method of any one of the preceding embodiments, wherein the ST2 antagonist is administered to the patient in combination with ICS≥500 mcg/day fluticasone propionate dose-equivalent plus LAMA plus LABA.

Embodiment 84. The method of any one of the preceding embodiments, which is associated with acceptable safety outcome compared with standard of care.

Embodiment 85. The method of embodiment 84, wherein the safety outcome is selected from any one or more of: incidence and severity of adverse events, with severity determined according to the Division of AIDS Table for Grading the Severity of Adult and Pediatric Adverse Events, Version 2.1 (DAIDS Table v2.1) toxicity scale; change from baseline in targeted vital signs; and/or change from baseline in targeted clinical laboratory test results and ECGs.

Embodiment 86. The method of any one of the preceding embodiments, wherein the patient is a former smoker.

Embodiment 87. The method of any one of embodiments 1-85, wherein the patient is a current smoker.

Embodiment 88. The method of any one of the preceding embodiments, wherein the patient has a baseline blood eosinophil count <300 eosinophils/μL.

Embodiment 89. The method of any one of the preceding embodiments, wherein the ST2 antagonist is an inhibitor of ST2 biological activity.

Embodiment 90. The method of any one of the preceding embodiments, wherein the ST2 antagonist binds to human ST2 or to human IL-33.

Embodiment 91. The method of any one of the preceding embodiments, wherein the ST2 antagonist is an anti-ST2 antibody.

Embodiment 92. The method of any one of the preceding embodiments, wherein the ST2 antagonist is astegolimab.

Embodiment 93. The method of embodiment 92, wherein the anti-ST2 antibody is a human antibody.

Embodiment 94. The method of embodiment 92 or embodiment 93, wherein the anti-ST2 antibody comprises:
a) heavy chain complementarity determining region (H-CDR) 1 comprising an amino acid sequence that is at least 90% identical to the amino acid sequence of SEQ ID NO: 1, H-CDR2 comprising an amino acid sequence that is at least 90% identical to the amino acid sequence of SEQ ID NO: 2 or SEQ ID NO: 31, H-CDR3 comprising an amino acid sequence that is at least 90% identical to the amino acid sequence of SEQ ID NO: 3, light chain complementarity determining region (L-CDR) 1 comprising an amino acid sequence that is at least 90% identical to the amino acid sequence of SEQ ID NO: 4, L-CDR2 comprising an amino acid sequence that is at least 90% identical to the amino acid sequence of SEQ ID NO: 5, and L-CDR3 comprising an amino acid sequence that is at least 90% identical to the amino acid sequence of SEQ ID NO: 6;
b) heavy chain complementarity determining region (H-CDR) 1 comprising an amino acid sequence that is at least 90% identical to the amino acid sequence of SEQ ID NO: 35, H-CDR2 comprising an amino acid sequence that is at least 90% identical to the amino acid sequence of SEQ ID NO: 36, H-CDR3 comprising an amino acid sequence that is at least 90% identical to the amino acid sequence of SEQ ID NO: 37, light chain complementarity determining region (L-CDR) 1 comprising an amino acid sequence that is at least 90% identical to the amino acid sequence of SEQ ID NO: 38, L-CDR2 comprising an amino acid sequence that is at least 90% identical to the amino acid sequence of SEQ ID NO: 39, and L-CDR3 comprising an amino acid sequence that is at least 90% identical to the amino acid sequence of SEQ ID NO: 40;
c) heavy chain complementarity determining region (H-CDR) 1 comprising an amino acid sequence that is at least 90% identical to the amino acid sequence of SEQ ID NO: 11, H-CDR2 comprising an amino acid sequence that is at least 90% identical to the amino acid sequence of SEQ ID NO: 12, H-CDR3 comprising an amino acid sequence that is at least 90% identical to the amino acid sequence of SEQ ID NO: 13, light chain complementarity determining region (L-CDR) 1 comprising an amino acid sequence that is at least 90% identical to the amino acid sequence of SEQ ID NO: 14, L-CDR2 comprising an amino acid sequence that is at least 90% identical to the amino acid sequence of SEQ ID NO: 15, and L-CDR3 comprising an amino acid sequence that is at least 90% identical to the amino acid sequence of SEQ ID NO: 16; or
d) heavy chain complementarity determining region (H-CDR) 1 comprising an amino acid sequence that is at least 90% identical to the amino acid sequence of SEQ ID NO: 21, H-CDR2 comprising an amino acid sequence that is at least 90% identical to the amino acid sequence of SEQ ID NO: 22, H-CDR3 comprising an amino acid sequence that is at least 90% identical to the amino acid sequence of SEQ ID NO: 23, light chain complementarity determining region (L-CDR) 1 comprising an amino acid sequence that is at least 90% identical to the amino acid sequence of SEQ ID NO: 24, L-CDR2 comprising an amino acid sequence that is at least 90% identical to the amino acid sequence of SEQ ID NO: 25, and L-CDR3 comprising an amino acid sequence that is at least 90% identical to the amino acid sequence of SEQ ID NO: 26.

Embodiment 95. The method of embodiment 92 or embodiment 93, wherein the anti-ST2 antibody comprises:
a) heavy chain complementarity determining region (H-CDR) 1 comprising the amino acid sequence of SEQ ID NO: 1, H-CDR2 comprising the amino acid sequence of SEQ ID NO: 2 or SEQ ID NO: 31, H-CDR3 comprising the amino acid sequence of SEQ ID NO: 3, light chain complementarity determining region (L-CDR) 1 comprising the amino acid sequence of SEQ ID NO: 4, L-CDR2 comprising the amino acid sequence of SEQ ID NO: 5, and L-CDR3 comprising the amino acid sequence of SEQ ID NO: 6;
b) heavy chain complementarity determining region (H-CDR) 1 comprising the amino acid sequence of SEQ ID NO: 35, H-CDR2 comprising the amino acid sequence of SEQ ID NO: 36, H-CDR3 comprising the amino acid sequence of SEQ ID NO: 37, light chain complementarity determining region (L-CDR) 1 comprising the amino acid sequence of SEQ ID NO: 38, L-CDR2 comprising the amino acid sequence of SEQ ID NO: 39, and L-CDR3 comprising the amino acid sequence of SEQ ID NO: 40;
c) heavy chain complementarity determining region (H-CDR) 1 comprising the amino acid sequence of SEQ ID NO: 11, H-CDR2 comprising the amino acid sequence of SEQ ID NO: 12, H-CDR3 comprising the amino acid sequence of SEQ ID NO: 13, light chain complementarity determining region (L-CDR) 1 comprising the amino acid sequence of SEQ ID NO: 14, L-CDR2 comprising the amino acid sequence of SEQ ID NO: 15, and L-CDR3 comprising the amino acid sequence of SEQ ID NO: 16; or
d) heavy chain complementarity determining region (H-CDR) 1 comprising the amino acid sequence of SEQ ID NO: 21, H-CDR2 comprising the amino acid sequence of SEQ ID NO: 22, H-CDR3 comprising the amino acid sequence of SEQ ID NO: 23, light chain complementarity determining region (L-CDR) 1 comprising the amino acid sequence of SEQ ID NO: 24, L-CDR2 comprising the amino acid sequence of SEQ ID NO: 25, and L-CDR3 comprising the amino acid sequence of SEQ ID NO: 26.

Embodiment 96. The method of embodiment 92 or embodiment 93, wherein the anti-ST2 antibody comprises (a) heavy chain complementarity determining region (H-CDR) 1 comprising the amino acid sequence of SEQ ID NO: 1, H-CDR2 comprising the amino acid sequence of SEQ ID NO: 2 or SEQ ID NO: 31, H-CDR3 comprising the amino acid sequence of SEQ ID NO: 3, light chain complementarity determining region (L-CDR) 1 comprising the amino acid sequence of SEQ ID NO: 4, L-CDR2 comprising the amino acid sequence of SEQ ID NO: 5, and L-CDR3 comprising the amino acid sequence of SEQ ID NO: 6; or (b) heavy chain complementarity determining region (H-CDR) 1 comprising the amino acid sequence of SEQ ID NO: 35, H-CDR2 comprising the amino acid sequence of SEQ ID NO: 36, H-CDR3 comprising the amino acid sequence of SEQ ID NO: 37, light chain complementarity determining region (L-CDR) 1 comprising the amino acid sequence of SEQ ID NO: 38, L-CDR2 comprising the amino acid sequence of SEQ ID NO: 39, and L-CDR3 comprising the amino acid sequence of SEQ ID NO: 40.

Embodiment 97. The method of any one of embodiments 92 to 96, wherein the anti-ST2 antibody comprises:
a) a heavy chain variable region comprising an amino acid sequence that is at least 90%, at least 95%, or at least 98% identical to the amino acid sequence of SEQ ID NO: 7 and a light chain variable region comprising an amino acid sequence that is at least 90%, at least 95%, or at least 98% identical to the amino acid sequence of SEQ ID NO: 8;
b) a heavy chain variable region comprising an amino acid sequence that is at least 90%, at least 95%, or at least 98% identical to the amino acid sequence of SEQ ID NO: 17 and a light chain variable region comprising an amino acid sequence that is at least 90%, at least 95%, or at least 98% identical to the amino acid sequence of SEQ ID NO: 18; or
c) a heavy chain variable region comprising an amino acid sequence that is at least 90%, at least 95%, or at least 98% identical to the amino acid sequence of SEQ ID NO: 27 and a light chain variable region comprising an amino acid sequence that is at least 90%, at least 95%, or at least 98% identical to the amino acid sequence of SEQ ID NO: 28.

Embodiment 98. The method of any one of embodiments 92 to 97, wherein the anti-ST2 antibody comprises:
a) a heavy chain variable region comprising the amino acid sequence of SEQ ID NO: 7 and a light chain variable region comprising the amino acid sequence of SEQ ID NO: 8;
b) a heavy chain variable region comprising the amino acid sequence of SEQ ID NO: 17 and a light chain variable region comprising the amino acid sequence of SEQ ID NO: 18; or
c) a heavy chain variable region comprising the amino acid sequence of SEQ ID NO: 27 and a light chain variable region comprising the amino acid sequence of SEQ ID NO: 28.

Embodiment 99. The method of any one of embodiments 92 to 97, wherein the anti-ST2 antibody comprises a heavy chain variable region comprising the amino acid sequence of SEQ ID NO: 7 and a light chain variable region comprising the amino acid sequence of SEQ ID NO: 8.

Embodiment 100. The method of any one of embodiments 92 to 99, wherein the anti-ST2 antibody comprises:
a) a heavy chain comprising an amino acid sequence that is at least 90%, at least 95%, or at least 98% identical to the amino acid sequence of SEQ ID NO: 9 or SEQ ID NO: 32 and a light chain comprising an amino acid sequence that is at least 90%, at least 95%, or at least 98% identical to the amino acid sequence of SEQ ID NO: 10;
b) a heavy chain comprising an amino acid sequence that is at least 90%, at least 95%, or at least 98% identical to the amino acid sequence of SEQ ID NO: 19 and a light chain comprising an amino acid sequence that is at least 90%, at least 95%, or at least 98% identical to the amino acid sequence of SEQ ID NO: 20; or
c) a heavy chain comprising an amino acid sequence that is at least 90%, at least 95%, or at least 98% identical to the amino acid sequence of SEQ ID NO: 29 and a light chain comprising an amino acid sequence that is at least 90%, at least 95%, or at least 98% identical to the amino acid sequence of SEQ ID NO: 30.

Embodiment 101. The method of any one of embodiments 92 to 100, wherein the anti-ST2 antibody comprises:
a) a heavy chain comprising the amino acid sequence of SEQ ID NO: 9 or SEQ ID NO: 32 and a light chain comprising the amino acid sequence of SEQ ID NO: 10;
b) a heavy chain comprising the amino acid sequence of SEQ ID NO: 19 and a light chain comprising the amino acid sequence of SEQ ID NO: 20; or
c) a heavy chain comprising the amino acid sequence of SEQ ID NO: 29 and a light chain comprising the amino acid sequence of SEQ ID NO: 30.

Embodiment 102. The method of any one of embodiments 92 to 101, wherein the anti-ST2 antibody comprises a heavy chain comprising the amino acid sequence of SEQ ID NO: 9 or SEQ ID NO: 32 and a light chain comprising the amino acid sequence of SEQ ID NO: 10.

Embodiment 103. A kit comprising an ST2 antagonist and instructions to administer the ST2 antagonist to a patient in accordance with the method of any one of embodiments 1-102.

Embodiment 104. An ST2 antagonist for use in a method of treating chronic obstructive pulmonary disease (COPD) in a patient comprising administering 476 mg of an ST2 antagonist to the patient on Day 1 of a treatment period.

Embodiment 105. An ST2 antagonist for use in a method of reducing the frequency of moderate to severe exacerbations in a patient having COPD comprising administering 476 mg of an ST2 antagonist to the patient on Day 1 of a treatment period.

Embodiment 106. An ST2 antagonist for use in a method of reducing the frequency of moderate to severe exacerbations in a patient having COPD comprising administering an effective amount of an ST2 antagonist to achieve a clinical improvement of at least 10%, at least 20%, at least 21%, at least 22%, at least 25%, at least 30%, at least 35%, at least 40% or at least 45% annualized exacerbation rate reduction than standard of care (SOC).

Embodiment 107. An ST2 antagonist for use in a method of reducing the frequency of moderate to severe exacerbations in a patient having COPD comprising administering an ST2 antagonist to the patient in an amount effective to achieve a greater clinical improvement in the number of exacerbations than standard of care (SOC), said patient having a baseline blood eosinophil count <300 eosinophils/μL.

Embodiment 108. An ST2 antagonist for use in a method of reducing the frequency of moderate to severe exacerbations in a patient having COPD comprising administering an ST2 antagonist to the patient in an amount effective to achieve a greater clinical improvement in the number of exacerbations than SOC, said patient having a baseline blood eosinophil count ≤170 eosinophils/μL.

Embodiment 109. An ST2 antagonist for use in a method of reducing the frequency of moderate to severe exacerbations in a patient having COPD comprising administering an ST2 antagonist to the patient in an amount effective to achieve a greater clinical improvement in the number of exacerbations than SOC, said patient having a post-bronchodilator (post-BD) spirometry measurement of <0.7 as measured by forced expiratory volume in one second (FEV1) and/or forced vital capacity (FVC).

Embodiment 110. An ST2 antagonist for use in a method of reducing the frequency of moderate to severe exacerbations in a patient having COPD comprising administering an ST2 antagonist to the patient in an amount effective to achieve a greater clinical improvement in the number of exacerbations than SOC, said patient having a modified Medical Research Council (mMRC) dyspnea scale score ≥2 and a COPD assessment test score (CAT) of ≥10.

Embodiment 111. An ST2 antagonist for use in a method of treating or preventing COPD comprising administering an ST2 antagonist to a patient in an amount effective to achieve a greater clinical improvement than SOC as measured by patient reported outcome (PRO), wherein the PRO is an improvement of at least about 1, at least about 2, at least about 3, or at least about 4 points from baseline in a St. George's Respiratory Questionnaire for COPD patients (SGRQ-C) at 4 weeks, 12 weeks, 24 weeks, 36 weeks, or 48 weeks from the start of treatment.

Embodiment 112. An ST2 antagonist for use in a method for maintaining and/or improving lung function in a patient having COPD comprising administering an ST2 antagonist to the patient in an amount effective to achieve a greater clinical improvement in lung function than SOC, wherein clinical improvement is demonstrated by a mean difference compared to baseline of at least 0.04 L, 0.05 L, 0.06 L, 0.07 L, 0.08 L, or 0.09 L as measured by post-BD FEV1 at 4 weeks, 12 weeks, 24 weeks, 36 weeks, or 48 weeks from the start of treatment.

Embodiment 113. An ST2 antagonist for use in a method of improving baseline blood eosinophil count in a patient having COPD comprising administering an ST2 antagonist to the patient in an amount effective to reduce mean blood eosinophil count by at least about 25%, e.g., at least about 30%, at least about 35%, at least about 40%, at least about 45% compared to baseline, after about 4 weeks, 12 weeks, 24 weeks, 36 weeks, or 48 weeks following administration of a first dose of an ST2 antagonist.

Embodiment 114. An ST2 antagonist for use in a method of improving baseline blood eosinophil count in a patient having COPD comprising administering an ST2 antagonist to the patient in an amount effective to reduce mean blood eosinophil count by at least about 25%, e.g., at least about 30%, at least about 35%, at least about 40%, at least about 45% compared to baseline, after about 4 weeks following administration of a first dose of an ST2 antagonist.

Embodiment 115. An ST2 antagonist for use in a method of reducing the frequency of moderate to severe exacerbations in a patient having COPD comprising administering an ST2 antagonist to the patient in an amount effective to achieve a reduction of at least about 25%, e.g., at least about 30%, at least about 35%, at least about 40%, or at least about 45% in the number of moderate to severe exacerbations at 50 weeks and/or 52 weeks from the start of treatment, as measured by annualized exacerbation rate as compared to SOC.

Embodiment 116. An ST2 antagonist for use in a method for maintaining and/or improving lung function in a patient having COPD comprising administering an ST2 antagonist to the patient in an amount effective to achieve a greater clinical improvement in lung function than SOC, wherein clinical improvement is demonstrated by a mean difference compared to baseline of at least about 5% as measured by post-BD FEV1 at 4 weeks, 12 weeks, 24 weeks, 36 weeks, or 48 weeks from the start of treatment.

Embodiment 117. An ST2 antagonist for use in a method of treating chronic obstructive pulmonary disease (COPD) in a patient comprising administering an effective amount of an ST2 antagonist to the patient, wherein the patient is selected for treatment on the basis of level of sST2 in a sample derived from the patient is determined to be at or above a reference level of sST2.

Embodiment 118. An ST2 antagonist for use in a method of reducing the frequency of moderate to severe exacerbations in a patient having COPD comprising administering an effective amount of an ST2 antagonist to the patient, wherein the patient is selected for treatment on the basis of the level of sST2 in a sample derived from the patient is determined to be at or above a reference level of sST2.

Embodiment 119. An ST2 antagonist for use in a method of treating COPD in a patient comprising administering an effective amount of an ST2 antagonist to the patient, wherein the patient is selected for treatment on the basis of the genotype of the patient determined to comprise a TT allele or CT allele at polymorphism rs10206753.

Embodiment 120. An ST2 antagonist for use in a method of reducing the frequency of moderate to severe exacerbations in a patient having COPD comprising administering an effective amount of an ST2 antagonist to the patient, wherein the patient is selected for treatment on the basis of the genotype of the patient determined to comprise a TT allele or CT allele at polymorphism rs10206753.

Embodiment 121. An ST2 antagonist for use in a method of treating COPD in a patient comprising administering an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient.

Embodiment 122. An ST2 antagonist for use in a method of reducing the frequency of moderate to severe exacerbations in a patient having COPD comprising administering an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient.

Embodiment 123. An ST2 antagonist for use in a method of treating COPD in a patient comprising administering an effective amount of an ST2 antagonist to the patient, wherein the patient is selected for treatment on the basis of the level of baseline α-diversity in a sample derived from the patient is determined to be below a reference level of alpha-diversity index.

Embodiment 124. An ST2 antagonist for use in a method of reducing the frequency of moderate to severe exacerbations in a patient having COPD comprising administering an effective amount of an ST2 antagonist to the patient, wherein the patient is selected for treatment on the basis of the level of baseline α-diversity in a sample derived from the patient is determined to be below a reference level of alpha-diversity.

Embodiment 125. The ST2 antagonist of any one of embodiments 106 to 124, wherein the use comprises administering 476 mg of the ST2 antagonist to the patient on Day 1 of a treatment period.

Embodiment 126. The ST2 antagonist of any one of embodiments 104 to 125, wherein the use comprises administering the ST2 antagonist every 4 weeks.

Embodiment 127. The ST2 antagonist of any one of embodiments 104 to 125, wherein the use comprises administering the ST2 antagonist every 2 weeks.

Embodiment 128. The ST2 antagonist of any one of embodiments 104 to 125, wherein the use comprises administering 476 mg of the ST2 antagonist every 4 weeks.

Embodiment 129. The ST2 antagonist of any one of embodiments 104 to 125, wherein the use comprises administering 476 mg of the ST2 antagonist every 2 weeks.

Embodiment 130. The ST2 antagonist of any one of embodiments 106 to 124, wherein the use comprises administering 490 mg of the ST2 antagonist.

Embodiment 131. The ST2 antagonist of any one of embodiments 106 to 124, wherein the use comprises administering 490 mg of the ST2 antagonist every 4 weeks.

Embodiment 132. The ST2 antagonist of any one of embodiments 106 to 124, wherein the use comprises administering 490 mg of the ST2 antagonist every 2 weeks.

Embodiment 133. The ST2 antagonist of any one of embodiments 104 to 132, wherein the ST2 antagonist is an inhibitor of ST2 biological activity.

Embodiment 134. The ST2 antagonist of any one of embodiments 102 to 133, wherein the ST2 antagonist binds to human ST2 or to human IL-33.

Embodiment 135. The ST2 antagonist of any one of embodiments 102 to 134, wherein the ST2 antagonist is an anti-ST2 antibody.

Embodiment 136. The ST2 antagonist of embodiment 135, wherein the anti-ST2 antibody is a human antibody.

Embodiment 137. The anti-ST2 antibody of embodiment 135 or embodiment 136, wherein the anti-ST2 antibody comprises:
a) heavy chain complementarity determining region (H-CDR) 1 comprising an amino acid sequence that is at least 90% identical to the amino acid sequence of SEQ ID NO: 1, H-CDR2 comprising an amino acid sequence that is at least 90% identical to the amino acid sequence of SEQ ID NO: 2 or SEQ ID NO: 31, H-CDR3 comprising an amino acid sequence that is at least 90% identical to the amino acid sequence of SEQ ID NO: 3, light chain complementarity determining region (L-CDR) 1 comprising an amino acid sequence that is at least 90% identical to the amino acid sequence of SEQ ID NO: 4, L-CDR2 comprising an amino acid sequence that is at least 90% identical to the amino acid sequence of SEQ ID NO: 5, and L-CDR3 comprising an amino acid sequence that is at least 90% identical to the amino acid sequence of SEQ ID NO: 6;
b) heavy chain complementarity determining region (H-CDR) 1 comprising an amino acid sequence that is at least 90% identical to the amino acid sequence of SEQ ID NO: 35, H-CDR2 comprising an amino acid sequence that is at least 90% identical to the amino acid sequence of SEQ ID NO: 36, H-CDR3 comprising an amino acid sequence that is at least 90% identical to the amino acid sequence of SEQ ID NO: 37, light chain complementarity determining region (L-CDR) 1 comprising an amino acid sequence that is at least 90% identical to the amino acid sequence of SEQ ID NO: 38, L-CDR2 comprising an amino acid sequence that is at least 90% identical to the amino acid sequence of SEQ ID NO: 39, and L-CDR3 comprising an amino acid sequence that is at least 90% identical to the amino acid sequence of SEQ ID NO: 40;
c) heavy chain complementarity determining region (H-CDR) 1 comprising an amino acid sequence that is at least 90% identical to the amino acid sequence of SEQ ID NO: 11, H-CDR2 comprising an amino acid sequence that is at least 90% identical to the amino acid sequence of SEQ ID NO: 12, H-CDR3 comprising an amino acid sequence that is at least 90% identical to the amino acid sequence of SEQ ID NO: 13, light chain complementarity determining region (L-CDR) 1 comprising an amino acid sequence that is at least 90% identical to the amino acid sequence of SEQ ID NO: 14, L-CDR2 comprising an amino acid sequence that is at least 90% identical to the amino acid sequence of SEQ ID NO: 15, and L-CDR3 comprising an amino acid sequence that is at least 90% identical to the amino acid sequence of SEQ ID NO: 16; or
d) heavy chain complementarity determining region (H-CDR) 1 comprising an amino acid sequence that is at least 90% identical to the amino acid sequence of SEQ ID NO: 21, H-CDR2 comprising an amino acid sequence that is at least 90% identical to the amino acid sequence of SEQ ID NO: 22, H-CDR3 comprising an amino acid sequence that is at least 90% identical to the amino acid sequence of SEQ ID NO: 23, light chain complementarity determining region (L-CDR) 1 comprising an amino acid sequence that is at least 90% identical to the amino acid sequence of SEQ ID NO: 24, L-CDR2 comprising an amino acid sequence that is at least 90% identical to the amino acid sequence of SEQ ID NO: 25, and L-CDR3 comprising an amino acid sequence that is at least 90% identical to the amino acid sequence of SEQ ID NO: 26.

Embodiment 138. The anti-ST2 antibody of embodiment 135 or embodiment 136, wherein the anti-ST2 antibody comprises:
a) heavy chain complementarity determining region (H-CDR) 1 comprising the amino acid sequence of SEQ ID NO: 1, H-CDR2 comprising the amino acid sequence of SEQ ID NO: 2 or SEQ ID NO: 31, H-CDR3 comprising the amino acid sequence of SEQ ID NO: 3, light chain complementarity determining region (L-CDR) 1 comprising the amino acid sequence of SEQ ID NO: 4, L-CDR2 comprising the amino acid sequence of SEQ ID NO: 5, and L-CDR3 comprising the amino acid sequence of SEQ ID NO: 6;
b) heavy chain complementarity determining region (H-CDR) 1 comprising the amino acid sequence of SEQ ID NO: 35, H-CDR2 comprising the amino acid sequence of SEQ ID NO: 36, H-CDR3 comprising the amino acid sequence of SEQ ID NO: 37, light chain complementarity determining region (L-CDR) 1 comprising the amino acid sequence of SEQ ID NO: 38, L-CDR2 comprising the amino acid sequence of SEQ ID NO: 39, and L-CDR3 comprising the amino acid sequence of SEQ ID NO: 40;
c) heavy chain complementarity determining region (H-CDR) 1 comprising the amino acid sequence of SEQ ID NO: 11, H-CDR2 comprising the amino acid sequence of SEQ ID NO: 12, H-CDR3 comprising the amino acid sequence of SEQ ID NO: 13, light chain complementarity determining region (L-CDR) 1 comprising the amino acid sequence of SEQ ID NO: 14, L-CDR2 comprising the amino acid sequence of SEQ ID NO: 15, and L-CDR3 comprising the amino acid sequence of SEQ ID NO: 16; or
d) heavy chain complementarity determining region (H-CDR) 1 comprising the amino acid sequence of SEQ ID NO: 21, H-CDR2 comprising the amino acid sequence of SEQ ID NO: 22, H-CDR3 comprising the amino acid sequence of SEQ ID NO: 23, light chain complementarity determining region (L-CDR) 1 comprising the amino acid sequence of SEQ ID NO: 24, L-CDR2 comprising the amino acid sequence of SEQ ID NO: 25, and L-CDR3 comprising the amino acid sequence of SEQ ID NO: 26.

Embodiment 139. The anti-ST2 antibody of embodiment 135 or embodiment 136, wherein the anti-ST2 antibody comprises (a) heavy chain complementarity determining region (H-CDR) 1 comprising the amino acid sequence of SEQ ID NO: 1, H-CDR2 comprising the amino acid sequence of SEQ ID NO: 2 or SEQ ID NO: 31, H-CDR3 comprising the amino acid sequence of SEQ ID NO: 3, light chain complementarity determining region (L-CDR) 1 comprising the amino acid sequence of SEQ ID NO: 4, L-CDR2 comprising the amino acid sequence of SEQ ID NO: 5, and L-CDR3 comprising the amino acid sequence of SEQ ID NO: 6; or (b) heavy chain complementarity determining region (H-CDR) 1 comprising the amino acid sequence of SEQ ID NO: 35, H-CDR2 comprising the amino acid sequence of SEQ ID NO: 36, H-CDR3 comprising the amino acid sequence of SEQ ID NO: 37, light chain complementarity determining region (L-CDR) 1 comprising the amino acid sequence of SEQ ID NO: 38, L-CDR2 comprising the amino acid sequence of SEQ ID NO: 39, and L-CDR3 comprising the amino acid sequence of SEQ ID NO: 40.

Embodiment 140. The anti-ST2 antibody of any one of embodiments 135 to 139, wherein the anti-ST2 antibody comprises:
a) a heavy chain variable region comprising an amino acid sequence that is at least 90%, at least 95%, or at least 98% identical to the amino acid sequence of SEQ ID NO: 7 and a light chain variable region comprising an amino acid sequence that is at least 90%, at least 95%, or at least 98% identical to the amino acid sequence of SEQ ID NO: 8;
b) a heavy chain variable region comprising an amino acid sequence that is at least 90%, at least 95%, or at least 98% identical to the amino acid sequence of SEQ ID NO: 17 and a light chain variable region comprising an amino acid sequence that is at least 90%, at least 95%, or at least 98% identical to the amino acid sequence of SEQ ID NO: 18; or
c) a heavy chain variable region comprising an amino acid sequence that is at least 90%, at least 95%, or at least 98% identical to the amino acid sequence of SEQ ID NO: 27 and a light chain variable region comprising an amino acid sequence that is at least 90%, at least 95%, or at least 98% identical to the amino acid sequence of SEQ ID NO: 28.

Embodiment 141. The anti-ST2 antibody of any one of embodiments 135 to 140, wherein the anti-ST2 antibody comprises:
a) a heavy chain variable region comprising the amino acid sequence of SEQ ID NO: 7 and a light chain variable region comprising the amino acid sequence of SEQ ID NO: 8;
b) a heavy chain variable region comprising the amino acid sequence of SEQ ID NO: 17 and a light chain variable region comprising the amino acid sequence of SEQ ID NO: 18; or
c) a heavy chain variable region comprising the amino acid sequence of SEQ ID NO: 27 and a light chain variable region comprising the amino acid sequence of SEQ ID NO: 28.

Embodiment 142. The anti-ST2 antibody of any one of embodiments 135 to 141, wherein the anti-ST2 antibody comprises a heavy chain variable region comprising the amino acid sequence of SEQ ID NO: 7 and a light chain variable region comprising the amino acid sequence of SEQ ID NO: 8.

Embodiment 143. The anti-ST2 antibody of any one of embodiments 135 to 142, wherein the anti-ST2 antibody comprises:
a) a heavy chain comprising an amino acid sequence that is at least 90%, at least 95%, or at least 98% identical to the amino acid sequence of SEQ ID NO: 9 or SEQ ID NO: 32 and a light chain comprising an amino acid sequence that is at least 90%, at least 95%, or at least 98% identical to the amino acid sequence of SEQ ID NO: 10;
b) a heavy chain comprising an amino acid sequence that is at least 90%, at least 95%, or at least 98% identical to the amino acid sequence of SEQ ID NO: 19 and a light chain comprising an amino acid sequence that is at least 90%, at least 95%, or at least 98% identical to the amino acid sequence of SEQ ID NO: 20; or
c) a heavy chain comprising an amino acid sequence that is at least 90%, at least 95%, or at least 98% identical to the amino acid sequence of SEQ ID NO: 29 and a light chain comprising an amino acid sequence that is at least 90%, at least 95%, or at least 98% identical to the amino acid sequence of SEQ ID NO: 30.

Embodiment 144. The anti-ST2 antibody of any one of embodiments 135 to 143, wherein the anti-ST2 antibody comprises:
a) a heavy chain comprising the amino acid sequence of SEQ ID NO: 9 or SEQ ID NO: 32 and a light chain comprising the amino acid sequence of SEQ ID NO: 10;
b) a heavy chain comprising the amino acid sequence of SEQ ID NO: 19 and a light chain comprising the amino acid sequence of SEQ ID NO: 20; or
c) a heavy chain comprising the amino acid sequence of SEQ ID NO: 29 and a light chain comprising the amino acid sequence of SEQ ID NO: 30.

Embodiment 145. The anti-ST2 antibody of any one of embodiments 135 to 144, wherein the anti-ST2 antibody comprises a heavy chain comprising the amino acid sequence of SEQ ID NO: 9 or SEQ ID NO: 32 and a light chain comprising the amino acid sequence of SEQ ID NO: 10.

### BRIEF DESCRIPTION OF THE DRAWINGS

FIG. 1 shows the distribution of the number of moderate-to-severe exacerbations among subjects by placebo and anti-ST2 treatment arms.

FIG. 2 shows the annualized exacerbation rate for all-comers for placebo or anti-ST2 treatment arm. For all-comers, the reduction in annualized rate of moderate/severe COPD exacerbations was 22% in those receiving astegolimab versus placebo.

FIG. 3A-3B show the annualized exacerbation rate per baseline blood eosinophil sub-groups for placebo and astegolimab treatment arms.

FIG. 4 shows the change from baseline of St. George's Respiratory Questionnaire-COPD (SGRQ-C) total score during the 48-weeks for all-comers. Astegolimab treatment demonstrated an improvement of SGRQ-C from baseline for placebo and anti-ST2 treatment arms.

FIG. 5A-5B show the SGRQ-C total scores based on the baseline blood eosinophil sub-groups for placebo and anti-ST2 treatment arms.

FIG. 6 shows the change from baseline in post-bronchodilator forced expiratory volume (post-BD FEV1) during the 48 weeks for all-comers for placebo and astegolimab treatment arms. Astegolimab treatment demonstrated a trend in improvement of FEV1.

FIG. 7A-7B show the baseline blood eosinophil sub-group analysis for post-BD FEV1 for placebo and anti-ST2 treatment ams.

FIG. 8A-8B show the change from baseline in blood eosinophil level over 48 weeks for placebo and anti-ST2 treatment arms.

FIG. 9 shows the change from baseline in % sputum eosinophil count for placebo and anti-ST2 treatment arms.

FIG. 10A-10C show frequency (FIG. 10A) or number per patient (FIG. 10B-10C) of certain adverse events, including serious adverse events, for treatment compared to placebo.

FIG. 11. Treatment effect (percent rate reduction, top) and annualized exacerbation rate per treatment arm (bottom) are plotted and faceted by IL1RL1 TIR domain tag SNP (rs10206753) genotype.

FIG. 12. ZENYATTA treatment effect (percent rate reduction, top) and annualized exacerbation rate per treatment arm (bottom) are plotted and faceted by pre-treatment categories of serum sST2 levels (< or ≥ median).

FIG. 13. ST2OP treatment effect (percent rate reduction, top) and annualized exacerbation rate per treatment arm (bottom) are plotted and faceted by pre-treatment categories of serum sST2 levels (< or ≥median).

FIG. 14. STEPP analysis was conducted by assessing the treatment effect for subpopulations defined by overlapping ranges of baseline serum sST2 as annotated in the plot margin. The annualized exacerbation rates are plotted.

FIG. 15. ST2OP treatment effect (percent rate reduction, top) and annualized exacerbation rate per treatment arm (bottom) are plotted and faceted by pre-treatment categories of baseline lung α-diversity (< or ≥median).

FIG. 16. STEPP analysis was conducted by assessing the treatment effect for subpopulations defined by overlapping ranges of baseline α-diversity as annotated in the plot margin. The annualized exacerbation rates are plotted.

### DETAILED DESCRIPTION OF THE CERTAIN EMBODIMENTS

ST2 is expressed on inflammatory cells including mast cells, basophils, innate lymphoid cells, T lymphocytes, and macrophages. IL33 is expressed at high levels in epithelial cells of mucosal tissues, particularly the lung, and acts as an ‘alarmin,’ being released upon inflammatory cell death, infection, or injury, to initiate innate immune responses. IL33 activity is elevated in multiple human respiratory diseases including asthma, COPD, IPF, and ARDS. Preclinical studies have shown that therapeutic IL33 inhibition is protective in pulmonary ARDS models, and ST2- or IL33-deficient mice exposed to cigarette smoke have decreased inflammatory responses in response to subsequent respiratory viral infections without compromising antiviral host defense.

An investigator initiated study (IIS) on the effect of an anti-ST2 antibody on chronic obstructive pulmonary disease showed that the anti-ST2 antibody was well tolerated and has potential efficacy in exacerbation reduction, lung function, and quality of life.

A Phase II study has been designed to investigate the effect of anti-ST2 antibody on chronic obstructive pulmonary disease. The study is a randomized, double-blind, placebo-controlled study. One study arm will be dosed with anti-ST2 antibody intravenously every two weeks. One study arm will be dosed with anti-ST2 antibody intravenously every four weeks. Another arm will receive placebo. All subjects will continue to receive the standard of care therapy that they were receiving at study entry.

### I. Definitions

Abbreviations that may be used in this description:

| Abbreviation | Definition |
|---|---|
| 5STS | five-repetition sit-to-stand (test) |
| ADA | anti-drug antibody |
| AER | annualized exacerbation rate |
| AERR | annualized exacerbation rate reduction |
| AHR | airway hyperreponsiveness |
| ATS | American Thoracic Society |
| AUC | area under the concentration-time curve |
| CAT ™ | COPD Assessment Test ™ |
| Cₘₐₓ | maximum concentration |
| COPD | chronic obstructive pulmonary disease |
| COVID-19 | coronavirus disease 2019 |
| CRO | contract research organization |
| DMC | Data Monitoring Committee |
| EC | Ethics Committee |
| eCRF | electronic Case Report Form |
| EDC | electronic data capture |
| eDiary | electronic diary |
| ERS | European Respiratory Society |
| E-RS ®:COPD | Evaluating Repiratory Symptoms in COPD |
| EXACT ® | EXAcerbations of Chronic Pulmonary Disease Tool |
| Fc | fragment crystallizable region |
| FEV1 | forced expiratory volume |
| FVC | forced vital capacity |
| HBA_{1C} | hemoglobin A_{1C} |
| HIPAA | Health Insurance Portability and Accountability Act |
| HRCT | high-resolution computed tomography |
| HRQoL | health-related quality of life |
| HV | healthy volunteer |
| ICH | International Council for Harmonisation |
| ICS | inhaled corticosteroid |
| iDMC | independent Data Monitoring Committee |
| IgG | immunoglobulin G |
| IIS | investigator-initiated study |
| IL-1 | interleukin-1 |
| IL-1RL1 | IL-1 receptor-like 1 |
| IL-1R AcP | interleukin 1 receptor accessory protein |
| IL-33 | interleukin-33 |
| IM | intramuscular |
| IMP | investigational medicinal product |
| IND | Investigational New Drug (Application) |
| IRB | Institutional Review Board |
| IV | Intravenous |
| LABA | long-acting beta-agonist |
| LAMA | long-activing muscarinic antagonist |
| LPLV | last patient last visit |
| MACE | major adverse cardiovascular event |
| MAD | multiple-ascending dose |
| mITT | modified intent-to-treat |
| mMRC | Modified Medical Research Council (dyspnea scale) |
| MN | mobile nursing |
| MTD | maximum tolerated dose |
| NGS | next-generation sequencing |
| NK | natural killer (cell) |
| OLE | open-label extension |
| PD | pharmacodynamic |
| PK | pharmacokinetic |
| PRO | patient-reported outcome |
| Q2W | every 2 weeks |
| Q4W | every 4 weeks |
| QTcF | QT interval corrected through use of Fridericia's formula |
| RBR | Research Biosample Repository |
| SAD | single-ascending dose |
| SAE | serious adverse event |
| SARS | severe acute respiratory syndrome |
| SARS-CoV-2 | SARS coronavirus-2 |
| SC | subcutaneous |
| SGRQ-C | St. George's Respiratory Questionnaire-COPD |
| SOC | standard of care |
| sST2 | secreted soluble form of ST2 |
| Th2 | T helper type 2 (cell) |
| ULN | upper limit of normal |
| WES | whole exome sequencing |
| WGS | whole genome sequencing |
| WHO | World Health Organization |

For the purposes herein “inflammation” refers to an immunological defense against infection, marked by increases in regional blood flow, immigration of white blood cells, and release of chemical toxins. Inflammation is one way the body uses to protect itself from infection. Clinical hallmarks of inflammation include redness, heat, swelling, pain, and loss of function of a body part. Systemically, inflammation may produce fevers, joint and muscle pains, organ dysfunction, and malaise.

The term “patient” herein refers to a human patient.

The term “chronic obstructive pulmonary disease,” abbreviated as COPD, refers to a pulmonary disorder characterized by coughing, sputum and dyspnea. COPD is a progressive disease, i.e., the severity of the symptoms usually increases with the duration of the disease. In most cases of COPD, especially in advanced cases, a cure is impossible. Therapy rather aims at slowing the progress of the disease and relief of the symptoms. In most cases, the exposure to inhaled toxins, often tobacco smoke, causes a chronic bronchitis. Chronic bronchitis leads to increased secretion of mucus, swelling of the mucosa and bronchospasm. The swelling associated with inflammation combined with the increased metabolic activity of the affected tissue lead to ischemia at the site of inflammation. The airflow in the lungs is, thus, obstructed. Occupational exposition to dust, isocyanates and fume from welding are also important causes of COPD. In non-smokers COPD may also be caused by a 1-antitrypsin deficiency.

In most cases COPD does not progress steadily, but rather shows periods of stable symptoms interrupted by periods of a sudden worsening of the disease, the acute exacerbations. Thus, in some embodiments, the COPD is accompanied by acute exacerbations.

In some embodiments, COPD is defined as FEV1/FVC ratio of <0.7 and bronchodilator response <12%.

The term “exacerbation” refers to episodes of new or progressive increase in shortness of breath, cough (changes in sputum production and/or sputum quality and/or cough frequency and/or increased dyspnea), wheezing, chest tightness, nocturnal awakenings ascribed to one of the symptoms above or a combination of these symptoms. The severity of an exacerbation ranges from mild to life-threatening and can be evaluated based on both symptoms and lung function. In some embodiments, an exacerbation is a COPD exacerbation, which is an acute worsening of respiratory symptoms that may result in additional therapy (GOLD 2021).

The term “acute exacerbation” refers to COPD exacerbations triggered by bacterial or viral infection of the airways or by environmental pollutants. Inflammation is increased during acute exacerbations. An acute exacerbation of COPD typically lasts for several days. Airway inflammation is increased during the exacerbation resulting in increased hyperinflation, reduced expiratory air flow and worsening of gas transfer.

The term “moderate exacerbation” refers to COPD exacerbations requiring treatment with corticosteroids and/or antibiotics.

The term “severe exacerbation” refers to COPD exacerbations requiring hospitalization or that lead to death.

The term “FEV1” refers to the volume of air exhaled in the first second of forced exhalation of air expellec from the lungs, starting from a position of maximum inspiration and with the subject making maximum effort. It is a measure of airway obstruction.

The term “FVC” or “forced vital capacity” refers to the total volume of air expelled from the lungs from a position of maximum inspiration with the subject making maximum effort.

The term “LABA” means long-acting beta-2 agonist, which agonist includes, for example, salmeterol, formoterol, bambuterol, albuterol, indacaterol, arforaioterol and cienbuterol.

The term “LAMA” means long-acting muscarinic antagonist, which agonists include, for example, tiotropium.

Examples of LABA/LAMA combinations include but are not limited to: olodaterol tiotropium (Boehringer Ingeiheim's) and indacaterol glycopyrronium (Novartis).

An “intravenous” or “iv” dose, administration, or formulation of a drug is one which is administered via a vein, e.g., by infusion.

A “subcutaneous” or “sc” dose, administration, or formulation of a drug is one which is administered under the skin, e.g., via a pre-filled syringe, auto-injector, or other device.

A “fixed dose” of a drug refers to a dose that is administered without regard to the patient's weight. In some embodiments, a fixed dose of an anti-ST2 antibody provided herein is 476 mg, 700 mg, 490 mg, 350 mg, or 280 mg dose.

For the purposes herein, “clinical status” refers to a patient's health condition. Examples include that the patient is improving or getting worse. In some embodiments, clinical status is based on an ordinal scale of clinical status. In some embodiments, clinical status is not based on whether or not the patient has a fever.

The term “patient-reported outcome” or “PRO” refers to instrument(s) completed to assess the treatment benefit and patient experience of Ab2. In some embodiments, the PRO includes SGRQ-C, mMRC, CAT, and/or EXACT.

The term “St. George's Respiratory Questionnaire-COPD” or “SGRQ-C” or “SGRQ” refers to a self-report questionnaire designed to measure the impact of COPD on health and well-being through the patient's perception of COPD-related experiences (Meguro et al. 2007). The SGRQ-C consists of 40 questions within 3 domains: symptoms (7 items), activity (13 items), and impacts (20 items). Each response has a unique, empirically derived weight. A total score is also produced. Lower scores of the SGRQ-C indicate better health-related quality of life. The SGRQ-C has no specific recall period, with the exception of one item that assesses the frequency of chest trouble attacks during the past year. The SGRQ-C takes approximately 10 minutes to complete.

The term “Modified Medical Research Council Dyspnea Scale” or “mMRC” refers to an assessment of activity-associated dyspnea via a single item that requires patients to select one of five statements best describing their level of breathlessness, with lower scores corresponding to less dyspnea impact. The mMRC has no specified recall period; it captures the patient's current status at the timepoint of administration.

The term “COPD Assessment Test” or “CAT” refers to a validated PRO that measures the impact of COPD on health status. The CAT is an 8-item questionnaire that includes items related to cough, phlegm, chest tightness, breathlessness ascending hills/stairs, activity limitation at home, confidence leaving home, sleep, and energy. The CAT uses a 6-point ordinal scale, ranging from 0 (no impairment) to 5 (maximum impairment), with a score range of 0-40 and higher scores indicating a greater disease impact. There is no specified recall period (questions are answered in reference to daily life); the questionnaire takes 1-2 minutes to complete.

The term “EXAcerbations of Chronic Pulmonary Disease Tool and Evaluating Respiratory Symptoms in COPD” or “EXACT” Questionnaire and the term “Evaluating Respiratory Symptoms in COPD” or “E-RS:COPD” Subset refer to a daily eDiary that assesses COPD exacerbations (Leidy et al. 2010). The 14-item questionnaire contains four domains: breathlessness (5 items), cough and sputum (3 items), chest symptoms (3 items), and additional attributes (3 items), which includes tiredness/weakness, sleep disturbance, and fear/worry. The EXACT has a recall period of “today”. The subset E-RS:COPD is composed of the breathlessness, cough and sputum, and chest symptoms domains of the EXACT (11 total items) and, as such, it specifically assesses COPD symptoms (Leidy et al. 2014). An E-RS:COPD total score is derived based on the three domains. The daily eDiary entry, including the EXACT plus a short-acting rescue medication question regarding rescue inhaler use, takes approximately 5 minutes to complete. Patients are asked to complete the diary every evening before going to bed.

An “ordinal scale” refers to a scale used to quantify outcomes which are non-dimensional. They can include an outcome at a single point in time or can examine change which has occurred between two points in time. In some embodiments, the two points of time are “Day 1” (when first dose of the ST2 antagonist is administered) compared with a later Day when the patient is evaluated and, optionally, at at a later Day when the patient is further evaluated. Ordinal scales include various “categories” which each evaluate patent status or outcome. In some embodiments, the ordinal scale is a “6-point ordinal scale”.

For the purposes herein, “standard of care” or “SOC” refers to treatments or drugs commonly used to treat patients with COPD including one of the following combinations of optimized, stable maintnenance therapy:
ICS≥500 mcg/day fluticasone propionate dose-equivalent plus long-acting betaagonist (LABA)
Long-acting muscarinic antagonist (LAMA) plus LABA
ICS≥500 mcg/day fluticasone propionate dose-equivalent plus LAMA plus LABA

In some embodiments, standard of care includes one of the following combinations of therapies: ICS+LABA, LAMA+LABA, or ICS+LAMA+LABA.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as methylprednisolone sodium succinate), dexamethasone or dexamethasone triamcinolone, hydrocortisone, and betamethasone. In some embodiments, the corticosteroid is selected from prednisone, methylprednisolone, hydrocortisone, and dexamethasone. In some embodiments, the corticosteroid is methylprednisolone. In some embodiments, the corticosteroid is “low-dose” glucocorticoid (e.g., ≤1-2 mg/kg/day methylprednisolone, e.g., for 3-5 days).

A nucleotide position in a genome at which more than one sequence is possible in a population is referred to herein as a “polymorphism” or “polymorphic site.” A polymorphic site may be a nucleotide sequence of two or more nucleotides, an inserted nucleotide or nucleotide sequence, a deleted nucleotide or nucleotide sequence, or a microsatellite, for example. A polymorphic site which is a single nucleotide in length is referred to herein as a single nucleotide polymorphism (SNP). When there are two, three or four alternative nucleotide sequences at a polymorphic site, each nucleotide sequence is referred to as a “polymorphic variant” or a “nucleic acid variant.” Each possible variant in the DNA sequence is referred to as an “allele.” Where two polymorphic variants exist, the polymorphic variant represented in a majority of samples from a population is referred to as a “prevalent allele,” or “major allele,” and the polymorpohic variant that is less prevalent in the population is referred to as an “uncommon allele” or “minor allele.”

The term “genotype” refers to a description of the alleles of a gene contained in an individual or a sample. In the context of this invention, no distinction is made between the genotype of an individual and the genotype of a sample originating from the individual.

Herein “human ST2” is a receptor also known as interleukin 1 receptor like 1 (IL1RL1). ST2 is expressed on inflammatory cells including mast cells, basophils, innate lymphoid cells, T lymphocytes, and macrophages, and its ligand, IL33, is expressed at high levels in epithelial cells of mucosal tissues, particularly the lung, and acts as an ‘alarmin,’ being released upon inflammatory cell death, infection, or injury, to initiate innate immune responses. Naturally occurring human ST2 variants are known and included in this definition. Human ST2 amino acid sequence information has been disclosed, see for example, UniProtKB/Swiss-Prot Q01638.4. Human IL-33 amino acid sequence has been disclosed, see for example, UniProtKB/Swiss-Prot: 095760.1.

An “ST2 antagonist” refers to agent that inhibits or blocks ST2 biological activity. In some embodiments, the ST2 antagonist inhibits or blocks ST2 biological activity via binding to human ST2 or human IL-33. In some embodiments, the ST2 antagonist is an antibody. In some embodiments, the ST2 antagonist is a monoclonal antibody that binds ST2. In some embodiments, the ST2 antagonist is a monoclonal antibody that binds IL-33.

A “neutralizing” anti-ST2 antibody herein is one which binds to ST2 and is able to inhibit, to a measurable extent, the ability of IL-33 to bind to and/or active ST2. Nonlimiting exemplary neutralizing anti-ST2 antibodies are provided herein.

A “native sequence” protein herein refers to a protein comprising the amino acid sequence of a protein found in nature, including naturally occurring variants of the protein. The term as used herein includes the protein as isolated from a natural source thereof or as recombinantly produced.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibody fragments” herein comprise a portion of an intact antibody which retains the ability to bind antigen. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example. Specific examples of monoclonal antibodies herein include chimeric antibodies, humanized antibodies, and human antibodies, including antigen-binding fragments thereof.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). Humanized antibodies herein specifically include “reshaped” antibodies as described in U.S. Pat. No. 5,795,965, expressly incorporated herein by reference.

A “human antibody” herein is one comprising an amino acid sequence structure that corresponds with the amino acid sequence structure of an antibody obtainable from a human B-cell, and includes antigen-binding fragments of human antibodies. Such antibodies can be identified or made by a variety of techniques, including, but not limited to: production by transgenic animals (e.g., mice) that are capable, upon immunization, of producing human antibodies in the absence of endogenous immunoglobulin production (see, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807)); selection from phage display libraries expressing human antibodies or human antibody fragments (see, for example, McCafferty et al., Nature 348:552-553 (1990); Johnson et al., Current Opinion in Structural Biology 3:564-571 (1993); Clackson et al., Nature, 352:624-628 (1991); Marks et al., J. Mol. Biol. 222:581-597 (1991); Griffith et al., EMBO J. 12:725-734 (1993); U.S. Pat. Nos. 5,565,332 and 5,573,905); generation via in vitro activated B cells (see U.S. Pat. Nos. 5,567,610 and 5,229,275); and isolation from human antibody producing hybridomas.

A “multispecific antibody” herein is an antibody having binding specificities for at least two different epitopes. Exemplary multispecific antibodies may bind to two different epitopes of ST2. Alternatively, an anti-ST2 binding arm may be combined with an arm that binds to a second antigen. Multispecific antibodies can be prepared as full-length antibodies or antibody fragments (e.g. F(ab′)₂ bispecific antibodies). Engineered antibodies with three or more (preferably four) functional antigen binding sites are also contemplated (see, e.g., US Appln. No. US 2002/0004587 A1, Miller et al.).

Antibodies herein include “amino acid sequence variants” with altered antigen-binding or biological activity. Examples of such amino acid alterations include antibodies with enhanced affinity for antigen (e.g. affinity matured antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al.); and/or increased or diminished serum half-life (see, for example, WO00/42072, Presta, L.).

The antibody herein may be conjugated with a “heterologous molecule” for example to increase half-life or stability or otherwise improve the antibody. For example, the antibody may be linked to one of a variety of non-proteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an exemplary embodiment of the invention.

The antibody herein may be a “glycosylation variant” such that any carbohydrate attached to the Fc region, if present, is altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) describing antibodies with modified glycosylation.

The term “hypervariable region” when used herein refers to the amino acid residues of an antibody that are responsible for antigen binding. In some embodiments, the hypervariable region comprises amino acid residues from a “complementarity determining region” or “CDR” (e.g. as determined by Kabat: residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (e.g. as determined by Chothia: residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some embodiments, the CDRs are determined according to IMGT (see, e.g., www.imgt.org/IMGTindex/CDR.php). “Framework” or “FR” residues are those variable domain residues other than the hypervariable region residues as herein defined.

A “full length antibody” is one which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof. Preferably, the full length antibody has one or more effector functions.

A “naked antibody” is an antibody (as herein defined) that is not conjugated to a heterologous molecule, such as a cytotoxic moiety, polymer, or radiolabel.

Antibody “effector functions” refer to those biological activities attributable to the Fc region (a native sequence Fc region or amino acid sequence variant Fc region) of an antibody. Examples of antibody effector functions include C1q binding, complement dependent cytotoxicity (CDC), Fc receptor binding, antibody-dependent cell-mediated cytotoxicity (ADCC), etc.

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgGl, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilis cells, etc.

As used herein, “specifically binding” or “binds specifically to” refers to an antibody selectively or preferentially binding to ST2 antigen. Preferably the binding affinity for antigen is of Kd value of 10⁻⁹ mol/l or lower (e.g. 10⁻¹⁰ mol/l), preferably with a Kd value of 10⁻¹⁰ mol/l or lower (e.g. 10⁻¹² mol/l). The binding affinity is determined with a standard binding assay, such as surface plasmon resonance technique (BIACORE®).

An “effective amount” or “therapeutically effective amount” of an agent, e.g., ST2 antagonist or a pharmaceutical formulation thereof, refers to an amount effective, at dosages and for periods of time necessary, to achieve the desired therapeutic or prophylactic result. For example, the expression “effective amount” in some embodiments refers to an amount of the ST2 antagonist that is effective for treating or preventing COPD. In some embodiments, an effective amount is 476 mg of an ST2 antagonist. In some embodiments, an effective amount is 476 mg SC every 2 weeks. In some embodiments, an effective amount is 476 SC every 4 weeks. In some embodiments, an effective amount is 490 mg SC every 4 weeks.

The term “pharmaceutical formulation” refers to a preparation which is in such form as to permit the biological activity of the active ingredient or ingredients to be effective, and which contains no additional components which are unacceptably toxic to a subject to which the formulation would be administered. Such formulations are sterile. In some embodiments, the formulation is for intravenous (iv) administration. In another embodiment, the formulation is for subcutaneous (sc) administration.

A “sterile” formulation is aseptic or free from all living microorganisms and their spores.

A “liquid formulation” or “aqueous formulation” according to the invention denotes a formulation which is liquid at a temperature of at least about 2 to about 8° C.

The term “lyophilized formulation” denotes a formulation which is dried by freezing the formulation and subsequently subliming the ice from the frozen content by any freeze-drying methods known in the art, for example commercially available freeze-drying devices. Such formulations can be reconstituted in a suitable diluent, such as water, sterile water for injection, saline solution etc., to form a reconstituted liquid formulation suitable for administration to a subject.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

An “elevated” level of a biomarker refers to an amount of that biomarker in the patient that is above the upper limit of normal (ULN).

### II. ST2 Antagonists

ST2 antagonists contemplated herein include antagonists that bind to ST2 or its ligand, IL-33.

In some embodiments, the ST2 antagonist is an antibody.

In some embodiments, the ST2 antagonist is an antibody that binds ST2.

In some embodiments, the ST2 antagonist blocks the IL-33/ST2 receptor complex.

In some embodiments, the ST2 antagonist blocks IL-33-mediated ST2 signaling.

Antibodies that bind ST2 include the human anti-ST2 antibodies described in WO 2013/173761 A2, which is incorporated by reference herein in its entirety for any purpose. Nonlimiting such antibodies include Ab2, Ab5, and Ab7, the sequences for which are provided in the Table of Certain Sequences herein. In some embodiments, the CDRs are determined according to Kabat. In some embodiments, the CDRs are determined according to IMGT.

Amino acid sequences of the heavy chain and light chain of anti-ST2 antibody Ab2 are shown in SEQ ID NO: 9 (or SEQ ID NO: 32, which lacks the C-terminal lysine) and SEQ ID NO: 10, respectively. The amino acid sequences of the heavy chain variable domain and light chain variable domain of anti-ST2 antibody Ab2 are shown in SEQ ID NOs: 7 and 8, respectively. The amino acid sequences of the heavy chain complementarity determining regions H-CDR1, H-CDR2, H-CDR3, and the light chain complementarity determining regions L-CDR1, L-CDR2, and L-CDR3 of anti-ST2 antibody Ab2 are shown in SEQ ID NOs: 1, 2 or 31, 3, 4, 5, and 6, respectively, for example, as determined by Kabat. The amino acid sequences of the heavy chain complementarity determining regions H-CDR1, H-CDR2, H-CDR3, and the light chain complementarity determining regions L-CDR1, L-CDR2, and L-CDR3 of anti-ST2 antibody Ab2 are shown in SEQ ID NOs: 35, 36, 37, 38, 39, and 40, respectively, for example, as determined by IMGT.

The amino acid sequences of the heavy chain and light chain of anti-ST2 antibody Ab5 are shown in SEQ ID NOs: 19 (or SEQ ID NO: 33, which lacks the C-terminal lysine) and SEQ ID NO: 20, respectively. The amino acid sequences of the heavy chain variable domain and light chain variable domain of anti-ST2 antibody Ab5 are shown in SEQ ID NOs: 17 and 18, respectively. The amino acid sequences of the heavy chain complementarity determining regions H-CDR1, H-CDR2, H-CDR3, and the light chain complementarity determining regions L-CDR1, L-CDR2, and L-CDR3 of anti-ST2 antibody Ab5 are shown in SEQ ID NOs: 11, 12, 13, 14, 15, and 16, respectively.

The amino acid sequences of the heavy chain and light chain of anti-ST2 antibody Ab7 are shown in SEQ ID NOs: 29 (or SEQ ID NO: 34, which lacks the C-terminal lysine) and SEQ ID NO: 30, respectively. The amino acid sequences of the heavy chain variable domain and light chain variable domain of anti-ST2 antibody Ab7 are shown in SEQ ID NOs: 27 and 28, respectively. The amino acid sequences of the heavy chain complementarity determining regions H-CDR1, H-CDR2, H-CDR3, and the light chain complementarity determining regions L-CDR1, L-CDR2, and L-CDR3 of anti-ST2 antibody Ab7 are shown in SEQ ID NOs: 21, 22, 23, 24, 25, and 26, respectively.

In some embodiments, the ST2 antagonist is Ab2. The amino acid sequence of the Ab2 light chain is as follows (SEQ ID NO: 10):

| | | | | | |
|---|---|---|---|---|---|
| DIQMTQSPSS | LSASVGDRVT | ITCQASQDIS | NYLNWYQQKP | GKAPKLLIYD | 50 |
| ASNLETGVPS | RESGSGSGTD | FTFTISSLQP | EDIATYYCQQ | DDNFPLTEGG | 100 |
| GTKVEIKRTV | AAPSVFIFPP | SDEQLKSGTA | SVVCLLNNFY | PREAKVQWKV | 150 |
| DNALQSGNSQ | ESVTEQDSKD | STYSLSSTLT | LSKADYEKHK | VYACEVTHQG | 200 |
| LSSPVTKSEN | RGEC. | | | | 214 |

The amino acid sequence of the Ab2 heavy chain is as follows (SEQ ID NO: 9):

| | | | | | |
|---|---|---|---|---|---|
| EVOLVQSGAE | VKKPGESLKI | SCKGSGYSFT | NYWIGWVRQM | PGKGLEWMGI | 50 |
| IYPGNSDTRF | SPSFQGQVTI | SADKSITTAY | LQWSSLKASD | TAMYYCARHG | 100 |
| TSSDYYGLDV | WGQGTTVTVS | SASTKGPSVE | PLAPCSRSTS | ESTAALGCLV | 150 |
| KDYFPEPVTV | SWNSGALTSG | VHTFPAVLOS | SGLYSLSSVV | TVPSSNEGTQ | 200 |
| TYTCNVDHKP | SNTKVDKTVE | RKCCVECPPC | PAPPVAGPSV | FLFPPKPKDT | 250 |
| LMISRTPEVT | CVVVDVSHED | PEVQFNWYVD | GVEVHNAKTK | PREEQENSTF | 300 |
| RVVSVLTVVH | QDWLNGKEYK | CKVSNKGLPA | PIEKTISKTK | GQPREPQVYT | 250 |
| LPPSREEMTK | NQVSLTCLVK | GFYPSDIAVE | WESNGQPENN | YKTTPPMLDS | 400 |
| DGSFFLYSKL | TVDKSRWQQG | NVFSCSVMHE | ALHNHYTOKS | LSLSPGK. | 447 |

An alternative amino acid sequence of the Ab2 heavy chain is as follows (SEQ ID NO: 32), which lacks the C-terminal lysine:

| | | | | | |
|---|---|---|---|---|---|
| EVQLVQSGAE | VKKPGESLKI | SCKGSGYSFT | NYWIGWVRQM | PGKGLEWMGI | 50 |
| IYPGNSDTRF | SPSFQGQVTI | SADKSITTAY | LQWSSLKASD | TAMYYCARHG | 100 |
| TSSDYYGLDV | WGQGTTVTVS | SASTKGPSVE | PLAPCSRSTS | ESTAALGCLV | 150 |
| KDYFPEPVTV | SWNSGALTSG | VHTFPAVLQS | SGLYSLSSVV | TVPSSNEGTQ | 200 |
| TYTCNVDHKP | SNTKVDKTVE | RKCCVECPPC | PAPPVAGPSV | FLFPPKPKDT | 250 |
| LMISRTPEVT | CVVVDVSHED | PEVQENWYVD | GVEVHNAKTK | PREEQENSTF | 300 |
| RVVSVLTVVH | QDWLNGKEYK | CKVSNKGLPA | PIEKTISKTK | GQPREPQVYT | 250 |
| LPPSREEMTK | NQVSLTCLVK | GFYPSDIAVE | WESNGQPENN | YKTTPPMLDS | 400 |
| DGSFFLYSKL | TVDKSRWQQG | NVFSCSVMHE | ALHNHYTQKS | LSLSPG. | 446 |

In some embodiments, the ST2 antagonist is a monoclonal antibody that binds IL-33.

In a preferred embodiment, the methods and articles of manufacture of the present invention use, or incorporate, an antibody that binds to human ST2. ST2 antigen to be used for production of, or screening for, antibodies may be, e.g., a soluble form of ST2 or a portion thereof (e.g. the extracellular domain), containing the desired epitope. Alternatively, or additionally, cells expressing ST2 at their cell surface can be used to generate, or screen for, antibodies. Other forms of ST2 useful for generating antibodies will be apparent to those skilled in the art.

In some embodiments, the antibody is an antibody fragment, various such fragments being disclosed above.

In another embodiment, the antibody is an intact or full-length antibody. Depending on the amino acid sequence of the constant domain of their heavy chains, intact antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known. In a preferred embodiment, the anti-ST2 antibody is an IgG2a antibody.

Techniques for generating antibodies are known and examples provided above in the definitions section of this document. In a preferred embodiment, the antibody is a chimeric, humanized, or human antibody or antigen-binding fragment thereof. Preferably the antibody is a humanized full-length antibody.

Various techniques are available for determining binding of the antibody to ST2. One such assay is an enzyme linked immunosorbent assay (ELISA) for confirming an ability to bind to human ST2. According to this assay, plates coated with ST2 (e.g. recombinant sST2) are incubated with a sample comprising the anti-ST2 antibody and binding of the antibody to the sST2 is determined.

Preferably, the anti-ST2 antibody is neutralizes IL-33 activity, e.g. by inhibiting binding of IL-33 to ST2. An exemplary method for evaluating such inhibition is disclosed in WO 2013/173761 A2, for example. According to this method, the ability of the antibody to compete with IL-33 to ST2 is evaluated. For example, a plate is coated with ST2 (e.g. recombinant ST2 or sST2), a sample comprising the anti-ST2 antibody with labeled IL-33 is added, and the ability of the antibody to block binding of the labeled IL-33 to ST2 is measured. Alternatively, or additionally, the ability of an anti-ST2 antibody to inhibit the IL-33-mediated association of ST2 with coreceptor AcP is determined. See WO 2013/173761 A2.

Non-limiting examples of anti-ST2 antibodies herein include Ab2, Ab5, and Ab7 (WO 2013/173761 A2).

The antibody herein is preferably recombinantly produced in a host cell transformed with nucleic acid sequences encoding its heavy and light chains (e.g. where the host cell has been transformed by one or more vectors with the nucleic acid therein). The preferred host cell is a mammalian cell, most preferably a Chinese Hamster Ovary (CHO) cells.

### III. Pharmaceutical Formulations

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

The formulation herein may also contain more than one active compound as necessary, preferably those with complementary activities that do not adversely affect each other. The type and effective amounts of such medicaments depend, for example, on the amount of antibody present in the formulation, and clinical parameters of the subjects. Exemplary such medicaments are discussed below.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

In some embodiments, the formulation is suitable for intravenous (iv) infusion. In some embodiments, an iv formulation is a sterile, clear, colorless to pale yellow, preservative-free solution for further dilution prior to intravenous infusion with a pH of approximately 6.5. In some embodiments, the iv formulation is supplied in a single-dose vial.

In some embodiments, the formulation is suitable for subcutaneous (sc) administration. In some embodiments, a sc formulation is a sterile, clear, colorless to slightly yellowish, preservative-free, histidine buffered solution for subcutaneous use with a pH of approximately 6.0. In some embodiments, a sc formulation is supplied in a ready-to-use, single-dose 0.9 mL prefilled syringe (PFS) with a needle safety device, or a ready-to-use, single-dose 0.9 mL autoinjector.

Preferably the formulation is isotonic.

### IV. Therapeutic Uses of ST2 Antagonists

The invention provides a method of treating chronic obstructive pulmonary disease (COPD) in a patient comprising administering 476 mg of an ST2 antagonist to the patient on Day 1 of a treatment period. In some embodiments, a method of reducing the frequency of moderate to severe exacerbations in a patient having COPD is provided, comprising administering 476 mg of an ST2 antagonist to the patient on Day 1 of a treatment period.

The invention provides a method of treating or preventing frequency of moderate to severe exacerbations in a patient having COPD comprising administering an effective amount of an ST2 antagonist to achieve a clinical improvement of at least 10%, at least 20%, at least 21%, at least 22%, at least 25%, at least 30%, at least 35%, at least 40% or at least 45% annualized exacerbation rate reduction than standard of care (SOC). In some embodiments, the clinical improvement is at least 25% reduction in the number of exacerbations than SOC. In some embodiments, the clinical improvement is at least 35% reduction in the number of exacerbations than SOC. In some embodiments, the clinical improvement is at least 45% reduction in the number of exacerbations than SOC. In some embodiments, the clinical improvement is between 25% and 75% reduction in the number of exacerbations than SOC. In some embodiments, the clinical improvement is between 25% and 50% reduction in the number of exacerbations than SOC. In some embodiments, a method of treating or preventing frequency of moderate to severe exacerbations in a patient having COPD is provided, comprising administering an ST2 antagonist to the patient in an amount effective to achieve a greater clinical improvement in the number of exacerbations than standard of care (SOC), said patient having a baseline blood eosinophil count <300 eosinophils/μL. In some embodiments, a method of treating or preventing frequency of moderate to severe exacerbations in a patient having COPD is provided, comprising administering an ST2 antagonist to the patient in an amount effective to achieve a greater clinical improvement in the number of exacerbations than SOC, said patient having a baseline blood eosinophil count ≤170 eosinophils/μL. In some embodiments, a method of treating or preventing frequency of moderate to severe exacerbations in a patient having COPD is provided, comprising administering an ST2 antagonist to the patient in an amount effective to achieve a greater clinical improvement in the number of exacerbations than SOC, said patient having a post-bronchodilator (post-BD) spirometry measurement of <0.7 as measured by forced expiratory volume in one second (FEV1) and/or forced vital capacity (FVC). In some embodiments, a method of treating or preventing frequency of moderate to severe exacerbations in a patient having COPD is provided, comprising administering an ST2 antagonist to the patient in an amount effective to achieve a greater clinical improvement in the number of exacerbations than SOC, said patient having a modified Medical Research Council (mMRC) dyspnea scale score ≥2 and a COPD assessment test score (CAT) of ≥10. In some embodiments, a method of treating or preventing COPD comprising is provided, comprising administering an ST2 antagonist to a patient in an amount effective to achieve a greater clinical improvement than SOC as measured by patient reported outcome (PRO), wherein the PRO is an improvement of at least about 1, at least about 2, at least about 3, or at least about 4 points from baseline in a St. George's Respiratory Questionnaire for COPD patients (SGRQ-C) at 4 weeks, 12 weeks, 24 weeks, 36 weeks, or 48 weeks from the start of treatment. In some embodiments, a method for maintaining and/or improving lung function in a patient having COPD comprising is provided, comprising administering an ST2 antagonist to the patient in an amount effective to achieve a greater clinical improvement in lung function than SOC, wherein clinical improvement is demonstrated by a mean difference compared to baseline of at least 0.04 L, 0.05 L, 0.06 L, 0.07 L, 0.08 L, or 0.09 L as measured by post-BD FEV1 at 4 weeks, 12 weeks, 24 weeks, 36 weeks, or 48 weeks from the start of treatment. In some embodiments, a method of improving baseline blood eosinophil count in a patient having COPD is provided, comprising administering an ST2 antagonist to the patient in an amount effective to reduce mean blood eosinophil count by at least about 25%, e.g., at least about 30%, at least about 35%, at least about 40%, at least about 45% compared to baseline, after about 4 weeks, 12 weeks, 24 weeks, 36 weeks, or 48 weeks following administration of a first dose of an ST2 antagonist. In some embodiments, a method of improving baseline blood eosinophil count in a patient having COPD is provided, comprising administering an ST2 antagonist to the patient in an amount effective to reduce mean blood eosinophil count by at least about 25%, e.g., at least about 30%, at least about 35%, at least about 40%, at least about 45% compared to baseline, after about 4 weeks following administration of a first dose of an ST2 antagonist. In some embodiments, a method of treating or preventing the frequency of moderate to severe exacerbations in a patient having COPD is provided, comprising administering an ST2 antagonist to the patient in an amount effective to achieve a reduction of at least about 25%, e.g., at least about 30%, at least about 35%, at least about 40%, or at least about 45% in the number of moderate to severe exacerbations at 50 weeks and/or 52 weeks from the start of treatment, as measured by annualized exacerbation rate as compared to SOC. In some embodiments, a method for maintaining and/or improving lung function in a patient having COPD is provided, comprising administering an ST2 antagonist to the patient in an amount effective to achieve a greater clinical improvement in lung function than SOC, wherein clinical improvement is demonstrated by a mean difference compared to baseline of at least about 5% as measured by post-BD FEV1 at 4 weeks, 12 weeks, 24 weeks, 36 weeks, or 48 weeks from the start of treatment. The invention provides a method of treating COPD in a patient, comprising administering an effective amount of an ST2 antagonist to the patient, wherein the patient is selected for treatment on the basis of level of sST2 in a sample derived from the patient is determined to be at or above a reference level of sST2. In some embodiments, a method of reducing the frequency of moderate to severe exacerbations in a patient having COPD is provided, comprising administering an effective amount of an ST2 antagonist to the patient, wherein the patient is selected for treatment on the basis of the level of sST2 in a sample derived from the patient is determined to be at or above a reference level of sST2. In some embodiments, the reference level of sST2 is at least 1 ng/mL, 5 ng/mL, 10 ng/mL, 15 ng/mL, or 19 ng/mL. In some embodiments, the reference level of sST2 is at least 1 ng/mL. In some embodiments, the reference level of sST2 is at least 5 ng/mL. In some embodiments, the reference level of sST2 is at least 10 ng/mL. In some embodiments, the reference level of sST2 is at least 15 ng/mL. In some embodiments, the reference level of sST2 is at least 19 ng/mL. In some embodiments, the reference level of sST2 is at least 19.1 ng/mL.

The invention provides a method of treating COPD in a patient comprising administering an effective amount of an ST2 antagonist to the patient, wherein the patient is selected for treatment on the basis of the genotype of the patient determined to comprise a TT allele or CT allele at polymorphism rs10206753. In some embodiments, a method of reducing the frequency of moderate to severe exacerbations in a patient having COPD is provided, comprising administering an effective amount of an ST2 antagonist to the patient, wherein the patient is selected for treatment on the basis of the genotype of the patient determined to comprise a TT allele or CT allele at polymorphism rs10206753.

The invention provides a method of treating COPD in a patient comprising administering an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient. In some embodiments, a method of reducing the frequency of moderate to severe exacerbations in a patient having COPD is provided, comprising administering an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient.

The invention provides a method of treating COPD in a patient comprising administering an effective amount of an ST2 antagonist to the patient, wherein the patient is selected for treatment on the basis of the level of baseline α-diversity in a sample derived from the patient is determined to be below a reference level of alpha-diversity index. In some embodiments, a method of reducing the frequency of moderate to severe exacerbations in a patient having COPD is provided, comprising administering an effective amount of an ST2 antagonist to the patient, wherein the patient is selected for treatment on the basis of the level of baseline α-diversity in a sample derived from the patient is determined to be below a reference level of alpha-diversity. In some embodiments, the reference level of baseline α-diversity is an α-diversity index of about 3.4, as calculated by Shannon-Weaver method. In some embodiments, the reference level of baseline α-diversity is an α-diversity index is in the range of about 0 to 5 as calculated by Shannon-Weaver method. In some embodiments, the reference level of alpha-diversity is an α-diversity index is 10 as calculated by Shannon-Weaver method.

In various embodiments, the dose is 476 mg of the ST2 antagonist.

In various embodiments, the dose of the ST2 antagonist is administered every 4 weeks. In some embodiments, the dose of the ST2 antagonist is administered every 2 weeks.

In various embodiments, the dose is 476 mg of the ST2 antagonist every 4 weeks. In some embodiments, the dose is 476 mg of the ST2 antagonist every 2 weeks.

In some embodiments, the dose is 490 mg of the ST2 antagonist. In some embodiments, the dose is 490 mg of the ST2 antagonist every 4 weeks.

The invention provides methods of treating COPD with an ST2 antagonist, which achieves a greater improvement in clinical outcome than standard of care.

Methods for confirming the improvement in clinical outcome compared with SOC include, without limitation, reduction in COPD exacerbation frequency. In some embodiments, improvement comprises reduction in the frequency of moderate-to-severe exacerbations (health care utilisation resulting in treatment with systemic corticosteroids and/or antibiotics or hospitalisation or death due to COPD, respectively) in 48 weeks as add-on to standard of care. In some embodiments, an improvement comprises improvement in annualized rate of moderate and severe COPD exacerbations over the 52-week treatment period.

In some embodiments, methods for confirming the improvement in clinical outcome compared with SOC include, without limitation:
Time to first moderate or severe COPD exacerbation during the 52-week treatment Period
Absolute change from baseline in health-related quality of life (HRQoL), e.g., at Week 52, as assessed through the St. George's Respiratory Questionnaire-COPD (SGRQ-C) total score
Proportion of patients with improvement in HRQoL, defined as a decrease from baseline of ≥4 points in SGRQ-C total score, e.g., at Week 52
Absolute change from baseline in post-bronchodilator FEV1 (liters), e.g., at Week 52
Absolute change from baseline in Evaluating Respiratory Symptoms in COPD (E-RS®:COPD) total score, e.g., at Week 52
Annualized rate of severe COPD exacerbations, e.g., over the 52-week treatment period
Absolute change from baseline in five-repetition sit-to-stand test (5STS) time (seconds), e.g., at Week 52

In some embodiments, methods for confirming the improvement in clinical outcome compared with SOC include, without limitation:
Annualized rate of EXAcerbations of Chronic Pulmonary Disease Tool (EXACT®)-defined exacerbation events, e.g., over the 52-week treatment period
An EXACT exacerbation event is defined as an EXACT total score of ≥12 points sustained for 2 days or ≥9 points sustained for 3 days.
Proportion of patients with HRQoL improvement, defined as a decrease from baseline of ≥4 points in SGRQ-C total score, e.g., at Week 12 and Week 24
Proportion of patients with symptom improvement, defined as a decrease of ≥2 points from baseline in E-RS:COPD total score, e.g., at Week 24 and Week 52
Absolute change from baseline in post-bronchodilator FEV1 (liters), e.g., at Weeks 12, 24, and 36
Absolute change from baseline in 5STS time (seconds), e.g., at Week 24
Annualized rate of moderate COPD exacerbations, e.g., over the 52-week treatment period
Duration of hospital stay for severe COPD exacerbations
Proportion of severe COPD exacerbations requiring hospital readmission, e.g., within 30 days
Absolute change from baseline in residual volume/total lung capacity ratio, e.g., at Week 52
Absolute change from baseline in daily step count, e.g., at Weeks 12, 24, and 52
Absolute change from baseline in time in moderate and vigorous physical activity, e.g., at Weeks 12, 24, and 52
Absolute change from baseline in COPD Assessment Test (CAT) score, e.g., at Week 52
Annualized rate of moderate and severe COPD exacerbations, e.g., over the blinded treatment period

In some embodiments, the method of treatment with the ST2 antagonist is associated with acceptable safety outcome compared with standard of care. Exemplary safety outcomes include any one or more of:
Incidence and severity of adverse events, with severity determined according to the Division of AIDS Table for Grading the Severity of Adult and Pediatric Adverse Events, Version 2.1 (DAIDS Table v2.1) toxicity scale
Change from baseline in targeted vital signs
Change from baseline in targeted clinical laboratory test results and ECGs

In another embodiment of any of the methods herein, the patient is treated with SOC along with the ST2 antagonist. SOC is disclosed above, and includes, for example, combination with inhaled corticosteroids. In some embodiments, standard of care includes ICS≥500 mcg/day fluticasone propionate dose-equivalent. In some embodiments, standard of care includes ICS plus long-acting betaagonist (LABA). In some embodiments, standard of care includes ICS≥500 mcg/day fluticasone propionate dose-equivalent plus LABA. In some embodiments, standard of care includes Long-acting muscarinic antagonist (LAMA) plus LABA. In some embodiments, standard of care includes ICS plus LAMA plus LABA. In some embodiments, standard of care includes ICS≥500 mcg/day fluticasone propionate dose-equivalent plus LAMA plus LABA.

In some embodiments, the ST2 antagonist binds ST2. In some embodiments, the ST2 antagonist binds IL-33. In some embodiments, the ST2 antagonist is an anti-ST2 antibody.

In some embodiments, the ST2 antagonist is Ab2, Ab5, or Ab7.

In another embodiment, the invention provides a method of treating COPD in a patient comprising administering an ST2 antagonist (e.g., an anti-ST2 antibody such as Ab2, Ab5, or Ab7) to the patient.

These additional drugs as set forth herein are generally used in the same dosages and with administration routes as used hereinbefore or about from 1 to 99% of the heretofore-employed dosages. If such additional drugs are used at all, preferably, they are used in lower amounts than if the first medicament were not present, especially in subsequent dosings beyond the initial dosing with the first medicament, so as to eliminate or reduce side effects caused thereby.

The combined administration of an additional drug includes co-administration (concurrent administration), using separate formulations or a single pharmaceutical formulation, and consecutive administration in either order, wherein preferably there is a time period while both (or all) active agents (medicaments) simultaneously exert their biological activities.

### V. Articles of Manufacture

In another embodiment of the invention, articles of manufacture containing materials useful for the treatment of COPD described above are provided.

The article of manufacture optionally further comprises a package insert with instructions for treating COPD in a subject, wherein the instructions indicate that treatment with the antibody as disclosed herein treats the COPD.

Further details of the invention are illustrated by the following non-limiting Examples. The disclosures of all citations in the specification are expressly incorporated herein by reference.

### Example 1: A Randomised Placebo-Controlled Trial of Anti-ST2 in COPD (COPD-ST2OP)

This is a single-centre, double-blinded, placebo-controlled, parallel group, randomized controlled trial comparing MSTT1041A (astegolimab, Ab2, anti-ST2 antibody) versus placebo in COPD. MSTT1041A 490 mg subcutaneous (s/c) or matched placebo dosed every 4 weeks for a total of 12 doses. Patients will be followed up for 60 weeks (i.e. 48 week treatment period and 12 week follow-up), with secondary outcome measures at baseline, 4, 12, 24, 36, 48 and 60 weeks and at exacerbations events presenting prior to treatment initiation. The dose and dosing interval has been derived from an earlier PK/PD modelling and is the highest dose included in an ongoing phase 2b asthma study. The primary outcome measure is exacerbation frequency. Exacerbation events are relatively infrequent and can be affected by season, therefore we have chosen a 48 week treatment duration with follow-up out to 12 months.

### Primary Objective

We hypothesise that anti-ST2 will impact on airway inflammation in COPD and consequently will reduce COPD exacerbation frequency.

The primary objective of the trial is to evaluate the efficacy of anti-ST2 versus placebo on frequency of moderate-to-severe exacerbations (health care utilisation resulting in treatment with systemic corticosteroids and/or antibiotics or hospitalisation or death due to COPD, respectively) in 48 weeks as add-on to standard of care.

### Secondary Objectives

Another key objective is to assess the safety and tolerability of subcutaneous doses of anti-ST2 compared to placebo in adult patients with moderate to very severe COPD.

Additionally, to assess the effects of anti-ST2 versus placebo both during stable visits and at the exacerbation events on the following:
1. Symptoms
2. Health status
3. Lung function
4. Sputum airway inflammation
5. Upper airway inflammation
6. Systemic inflammation
7. Airway infection and ecology
8. Breath volatile organic compound profiling
9. Airway morphometry and lung densitometry
10. Pharmacogenomics
11. Pharmacokinetics and ADA level

### Outcome Measures

### Primary Outcome

The primary outcome is: Frequency of moderate to severe exacerbation (defined as requiring treatment with systemic corticosteroids and/or antibiotics in the community or hospital or hospitalisation) in 48 weeks.

Where a COPD exacerbation is defined by symptomatic worsening of COPD requiring:
Use of systemic corticosteroids for at least 3 days; a single depot injectable dose of corticosteroids will be considered equivalent to a 3-day course of systemic corticosteroids; and/or
Use of antibiotics; and/or
An inpatient hospitalisation or death due to COPD

### Secondary Outcomes

1. AE event rate per year in the 48 weeks of the trial from first dose
2. SAE event rate per year in the 48 weeks of the trial from first dose
3. Laboratory measures
4. Vital signs (pulse, BP, temp, Oxygen saturation (O2 sats))
5. Cardiac function:
   Echocardiogram (ECHO)
   12 lead Electrocardiogram (ECG)

(If, in the opinion of investigator, the ECG is grossly abnormal (e.g. Left bundle branch block (LBBB), prolonged QTc), it will be compared to old ECGs. If there are no old ECGs for comparison, the investigator will make a clinical judgement about the suitability of the patients to take part in trial.)
6. Lung function:
   Whole body plethysmography (body box) (to be done anytime between screening and week 12, unless patient has had the test within 12 months prior to screening visit)
   Pre and post BD Spirometry
   Post BD Forced Expiratory Volume in 1 Second (FEV1)
   Transfer factor
7. Sputum airway inflammation
   sputum cytology
   mediator profiling (biomarkers)
8. Upper airway inflammation:
   nasosorption
   nasal epithelial sampling
9. Systemic inflammation:
   blood inflammatory cell differentials
   mediators
   cell subset analysis including but not restricted to exploration of ILC2 cells
   urine biomarkers of inflammation
10. Airway infection and ecology:
targeted qPCR (bacteria and viruses) for common airway pathogens
   microbiomics
11. Breath volatile organic compound (VOC) profiling (PTRMS & ADVION)—breathomics
12. Airway morphometry and lung densitometry:
   Thoracic CT-derived outcomes (non-contrast CT Scan)
   Chest X-Ray (CXR)
13. Pharmacogenomics-response analysis in subgroups determined by SNPs for alleles associated with the IL33/ST2 axis.
14. Pharmacokinetics PK and ADA level
15. Questionnaires and Scores:
   SGRQ-c and CAT—to evaluate Health status
   mMRC Dyspnoea Scale—to evaluate respiratory symptoms
   Visual analogue score for dyspnoea, cough sputum production (100 mm)—to evaluate respiratory symptoms
   sputum purulence colour card—to evaluate respiratory symptoms
16. Blood Tests:
   Full Blood Count (FBC)
   Urea & electrolytes (U&Es)
   Liver Function Tests (LFTs)
   C-Reactive Protein (CRP)
   RNA (PAXgene)
   DNA (PAXgene)
   Total IgE & RAST (HDM, pollen, cat, dog)
   Serum/plasma for inflammatory biomarkers
   Lipid profile
   N-terminal pro b-type natriuretic peptide (NTproBNP)
   HbA1c
   Pharmacokinetics (PK) and anti-drug antibody (ADA)
   (Pk samples should be taken pre-dose at dosing visits)

### Trial Design

This is a single-centre, double-blind, placebo-controlled, parallel group, randomised controlled trial to assess the efficacy and safety of anti-ST2 compared to placebo, in patients with moderate to very severe COPD (GOLD II-IV). Anti-ST2 will be administered via subcutaneous injection once every 4 weeks (Week 0, 4, 8, 12, 16, 20, 24, 28, 32, 36, 40, and 44) during the 48-week treatment period. The treatment period will be followed by a 12-week follow-up period (i.e washout period).

After signing the informed consent at the initial visit, patients will enter a screening period within 7-14 days of randomisation. Patients who qualify to participate in the trial will be randomized into a 48-week treatment period in which they will receive either 490 mg anti-ST2 or a matching placebo. Patients will be evaluated for an additional 12 weeks following completion of the randomised treatment period. An interim analysis is planned when the last patient completes the 48-week treatment period. Treatment groups will remain blinded until the 48-week followup period is completed, and trial database is locked.

### Patient Eligibility Criteria

### Inclusion Criteria

1. Symptoms typical of COPD when stable (baseline mMRC dyspnoea score ≥2)
2. GOLD COPD stage 2-4
3. Smoking pack years ≥10 years
4. Age >40 years
5. Receiving standard-of-care drug therapy as per BTS guidance for COPD
6. A history of ≥2 moderate-to-severe exacerbations in the last 12 months.
7. Be able to give valid written consent; compliant with trial procedures and trial visits.
8. Able to understand written and spoken English

### Exclusion Criteria

1. Significant known respiratory disorders other than COPD that in the view of the investigator will affect the trial
2. Patients whose treatment is considered palliative (life expectancy <12 months)
3. Known hypersensitivity to the active substance of IMP or any of the excipients
4. Known history of anaphylaxis
5. Patients with a COPD exacerbation and/or pneumonia within the 4 weeks prior to visit 1
6. Have, in the opinion of investigator, uncontrolled co-morbid conditions, such as diabetes mellitus, hypertension and heart failure [e.g. NYHA class III (e.g. less than ordinary activity causes fatigue, palpitation, or dyspnoea) patients will be excluded if they have had exacerbation of their HF in previous 6 months, and class IV (e.g. Symptoms of heart failure at rest)] that will affect the trial.
7. Myocardial infarction, unstable angina or stroke within 12 month prior to screening
8. Diagnosis of malignancy within 5 years of visit 1 (except for excised localised carcinoma of skin not including malignant melanoma)
9. Clinically significant ECG changes, which in the opinion of investigator warrants further investigations
10. Laboratory abnormalities, which in the opinion of investigator warrants further investigations
11. Have, in the opinion of the investigator, evidence of alcohol, drug or solvent abuse.
12. Pregnant, breastfeeding, or lactating women. Women of child-bearing potential must have a negative blood serum pregnancy test performed at the screening visit and must agree to use two methods of birth control, (one of which must be a barrier method).
13. Participation in an interventional clinical trial within 3 months of visit 1 or receipt of any investigational medicinal product within 3 months or 5 half-lives.
14. Upon questioning the patient has blood born infection (e.g. HIV, hepatitis B or C).

### Formulation

Anti-ST2 is presented as sterile, clear, and colourless to slightly yellow liquid. Each sterile vial is filled with a 1 mL deliverable volume of 70 mg/mL. It is formulated with 15 mM sodium acetate, 9.0% (w/v) sucrose, 0.01% (w/v) polysorbate 20, pH 5.2.

Placebo for Anti-ST2 (MSTT1041A) is formulated with 10 mM sodium acetate, 9.0% (w/v) sucrose, 0.004% (w/v) polysorbate 20, pH 5.2, and is supplied in an identical vial configuration.

### Results and Conclusions

81 participants were randomised to the COPD-ST2OP trial. 39 participants were allocated to the placebo arm. 42 were allocated to the anti-ST2 (astegolimab) arm. All 81 patients received at least one dose according to their allocated treatment. In total 67 participants completed all the dosing visits (12 in total).

Patients presented with the characteristics shown in Tables 1-4.

TABLE 1
Clinical characteristics - demographics & exacerbation history

| | Astegolimab, | Placebo, | Total, |
|---|---|---|---|
| Characteristics | n = 42 | n = 39 | n = 81 |
| Age- year | 67.6 ± 8.2 | 70.8 ± 6.2 | 69.1 ± 7.5 |
| Male sex- no. (%) | 25 (59.5) | 26 (66.7) | 51 (63.0) |
| White race- no. (%) | 41 (97.6) | 39 (100.0) | 80 (98.8) |
| BMI | 27.5 ± 6.1 | 26.1 ± 4.9 | 26.8 ± 5.5 |
| Current | 10 (23.8) | 6 (15.4) | 16 (19.8) |
| Ex-smoker | 32 (76.2) | 33 (84.6) | 65 (80.2) |
| Cigarette consumption- pack | 57.8 ± 30 | 52.1 ± 23.3 | 55.1 ± 27 |
| year | | | |
| No. of exacerbations in previous | 3.1 ± 1.7 | 3.1 ± 1.5 | 3.1 ± 1.6 |
| 12 months | | | |
| 2 Exacerbations in previous 12 | 20 (47.6) | 20 (51.3) | 40 (49.4) |
| months, (%) | | | |
| 3 Exacerbations in previous 12 | 10 (23.8) | 7 (17.9) | 17 (21.0) |
| months, (%) | | | |
| ≥4 Exacerbations in previous | 12 (28.6) | 12 (30.8) | 24 (29.6) |
| 12 months, (%) | | | |

TABLE 2
Clinical characteristics - patient reported outcomes (PRO)

| | Astegolimab, | Placebo, | Total, |
|---|---|---|---|
| PROS | n = 42 | n = 39 | n = 81 |
| SGQR-c total score | 60.1 ± 13.7 | 58.4 ± 17.6 | 59.3 ± 15.6 |
| mMRC score | 2.2 ± 0.9 | 2.3 ± 0.8 | 2.2 ± 0.9 |
| CAT score | 22.1 ± 6.6 | 22.6 ± 5.7 | 22.4 ± 6.2 |
| VAS dyspnoea score (mm) | 52.5 ± 20.3 | 53.5 ± 22.1 | 53.0 ± 21.0 |
| VAS cough score (mm) | 49.7 ± 23.7 | 47.5 ± 22.7 | 48.7 ± 23.1 |
| VAS sputum score (mm) | 40.5 ± 25.5 | 42.8 ± 24.5 | 41.6 ± 24.9 |

TABLE 3
Clinical characteristics - lung function

| | Astegolimab, | Placebo, | Total, |
|---|---|---|---|
| Lung function | n = 42 | n = 39 | n = 81 |
| Post-BD FEV1, litre | 1.20 ± 0.5 | 1.10 ± 0.5 | 1.20 ± 0.5 |
| Post-BD FEV1, % predicted | 48.20 ± 17.9 | 44.90 ± 15.6 | 46.60 ± 16.8 |
| Post-BD FEV1/FVC ratio, % | 45.00 ± 12.5 | 43.70 ± 11.9 | 44.40 ± 12.1 |

TABLE 4
Clinical characteristics - inflammation

| | Astegolimab, | Placebo, | Total, |
|---|---|---|---|
| Inflammatory markers | n = 42 | n = 39 | n = 81 |
| Blood eosinophils | 220 | 160 | 170 |
| cells/μL, median [IQR] | [110, 300] | [120, 310] | [120, 300] |
| Blood neutrophils | 5.5 | 5.4 | 5.4 |
| cells/10⁹/L, median | [4.2, 7.4] | [4.0, 6.0] | [4.2, 6.3] |
| [IQR] | | | |
| Sputum eosinophils, % | 0.8 | 2.0 | 1.0 |
| median [IQR] | (0.0, 3.0) | (0.5, 4.0) | (0.3, 3.0) |
| Sputum neutrophils, % | 83.5 | 84.3 | 84.0 |
| median [IQR] | (55.5, 95.3) | (70.0, 92.0) | (69.0, 93.5) |

The primary outcome is frequency of moderate to severe exacerbation (defined as requiring treatment with systemic corticosteroids and/or antibiotics in the community or hospital or hospitalisation) in 48 weeks. Astegolimab treatment demonstrated numerical reductions in COPD exacerbations.

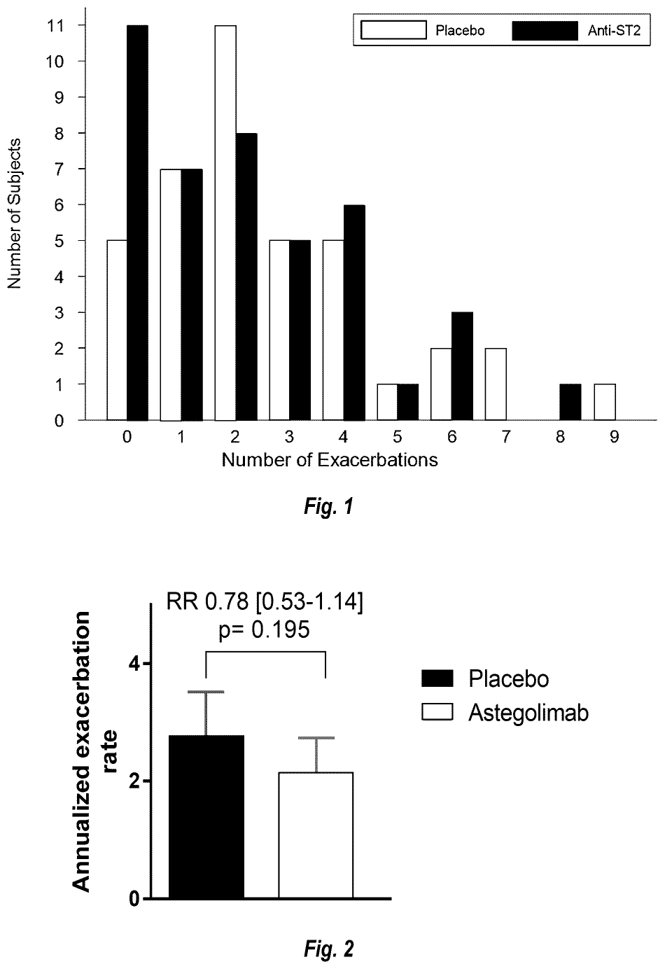

FIG. 1 shows the distribution of the number of moderate-to-severe exacerbations among subjects by treatment arm.

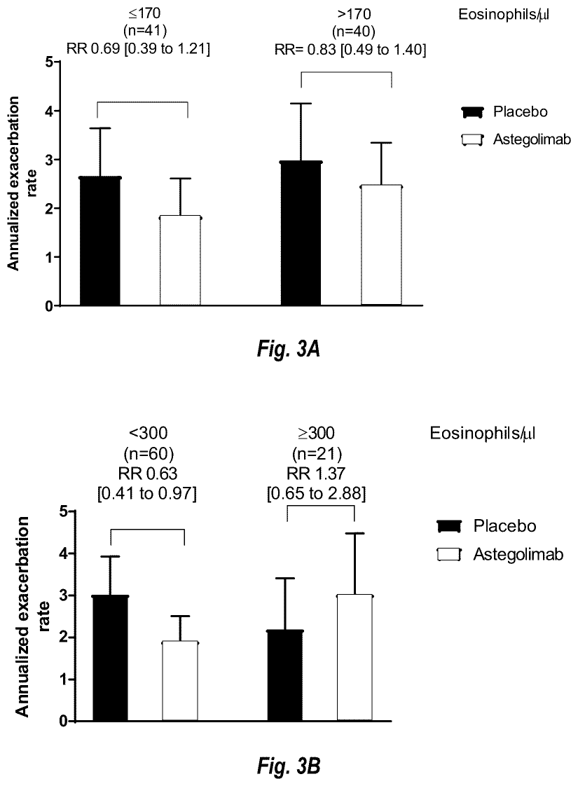

FIG. 2 shows the annualized exacerbation rate for all-comers, where the mean number of annualized moderate/severe COPD exacerbation for placebo and astegolimab over 48 weeks were 2.81 [2.05 to 3.58] and 2.18 [1.59 to 2.78], respectively. For all-comers, the reduction in annualized rate of moderate/severe COPD exacerbations was 22% in those receiving astegolimab versus placebo. FIGS. 3A-3B demonstrate the annualized exacerbation rate per baseline blood eosinophil sub-groups, having, respectively, baseline blood eosinophil counts of <170 eosinophils/μL and >170 eosinophils/μL, or <300 eosinophils/μL and ≥300 eosinophils/μL. As shown in FIG. 3A, the sub-group having baseline blood eosinophil ≤170 eosinophils/μL showed an AERR of 31% and the sub-group having baseline blood eosinophil >170 eosinophils/μL showed an AERR of 17%. FIG. 3B shows an AERR of 37% for the subgroup having a baseline blood eosinophil count of <300 eosinophils/μL.

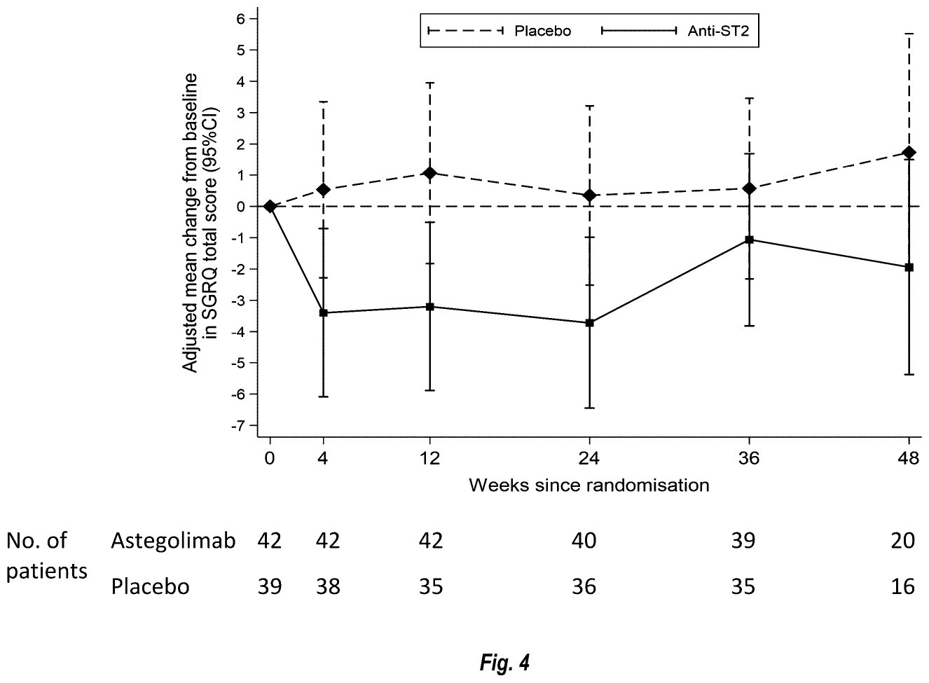

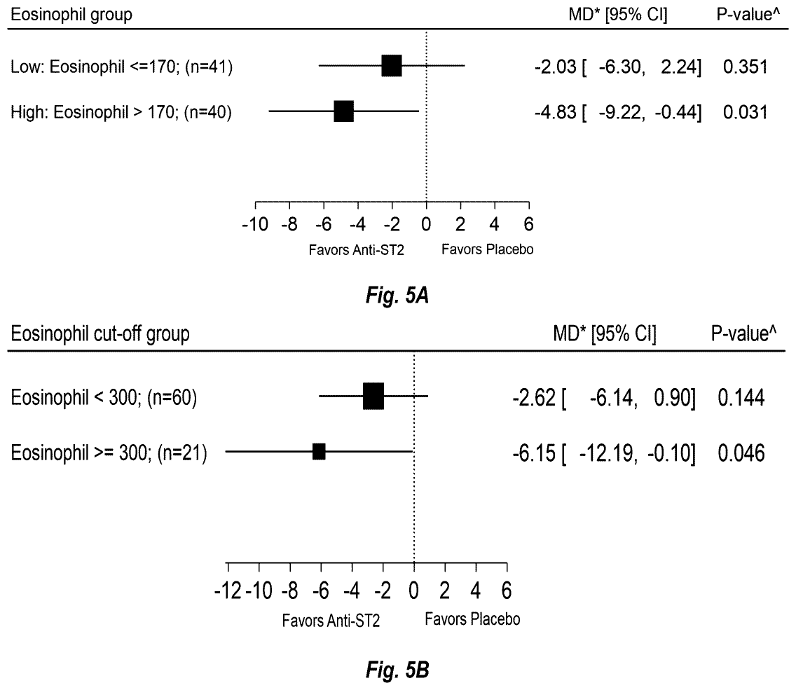

Astegolimab treatment also demonstrated an improvement of SGRQ-C from baseline. FIG. 4 shows the change from baseline in SGRQ total score during the 48-weeks for all-comers, where the adjusted mean SGRQ-C total score difference comparing astegolimab to placebo over 48 weeks=−3.3 (95% CI, −6.4 to −0.2; p=0.039). FIGS. 5A-5B show the SGRQ total scores based on the baseline blood eosinophil sub-groups for, respectively, eosinophil group having low (≤) or high (>) 170 eosinophils/μL (FIG. 5A) or < or ≥300 eosinophils/μL (FIG. 5B).

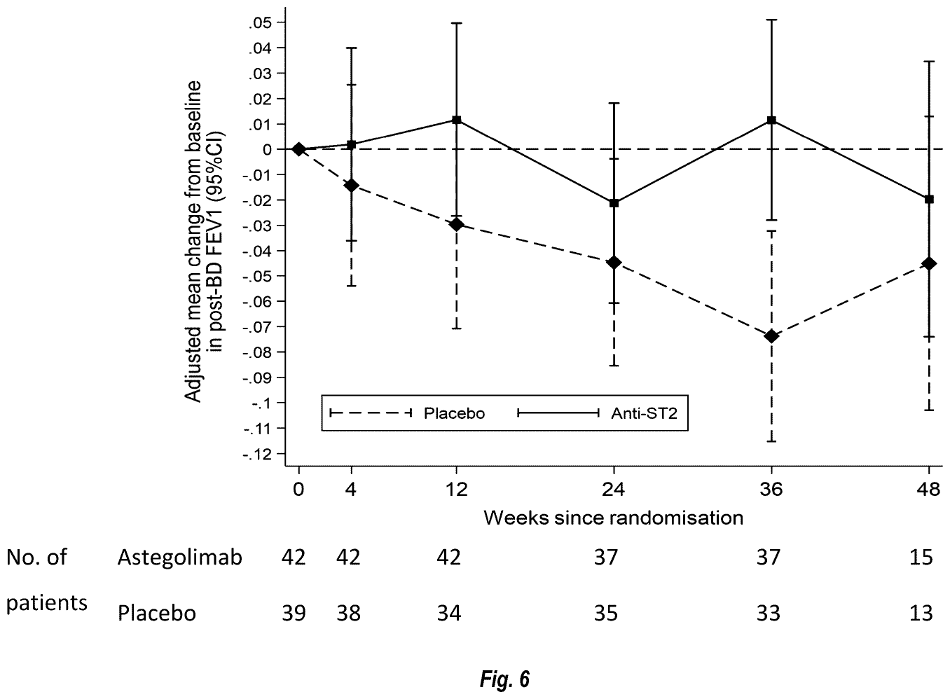

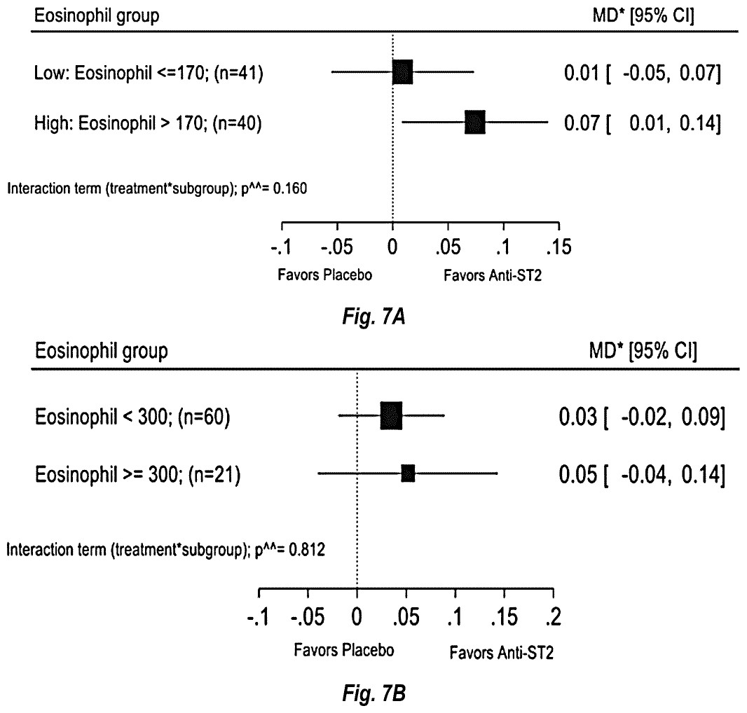

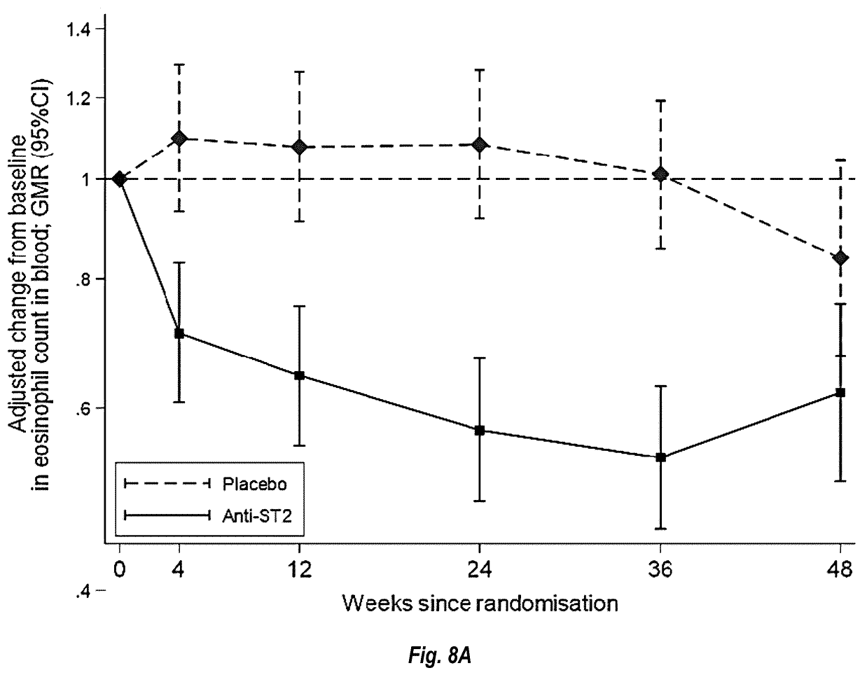

FIG. 6 shows the change from baseline in post-BD FEV1 during the 48 weeks for all-comers. The adjusted mean difference FEV1 (L) over 48 weeks comparing astegolimab to placebo was 0.04 L (95% CI, −0.01 to 0.09; p=0.094). FIGS. 7A-7B show the changes in post-BD FEV1 for baseline blood eosinophil sub-groups having, respectively, low (≤) or high (>) 170 eosinophils/μL (FIG. 7A) or < or ≥300 eosinophils/μL (FIG. 7B).

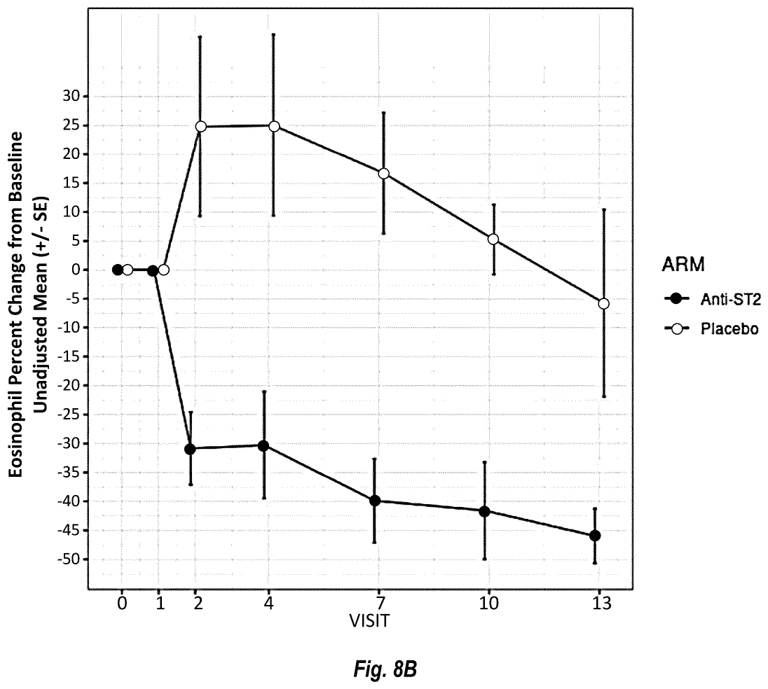

FIGS. 8A-8B show the change from baseline in blood eosinophil level over 48 weeks. The median blood eosinophil count was 170 cells/μL. The geometric mean ratio for the blood eosinophil count compared to placebo from baseline to week 48 was 0.61 (0.50 to 0.73; p<0.001).

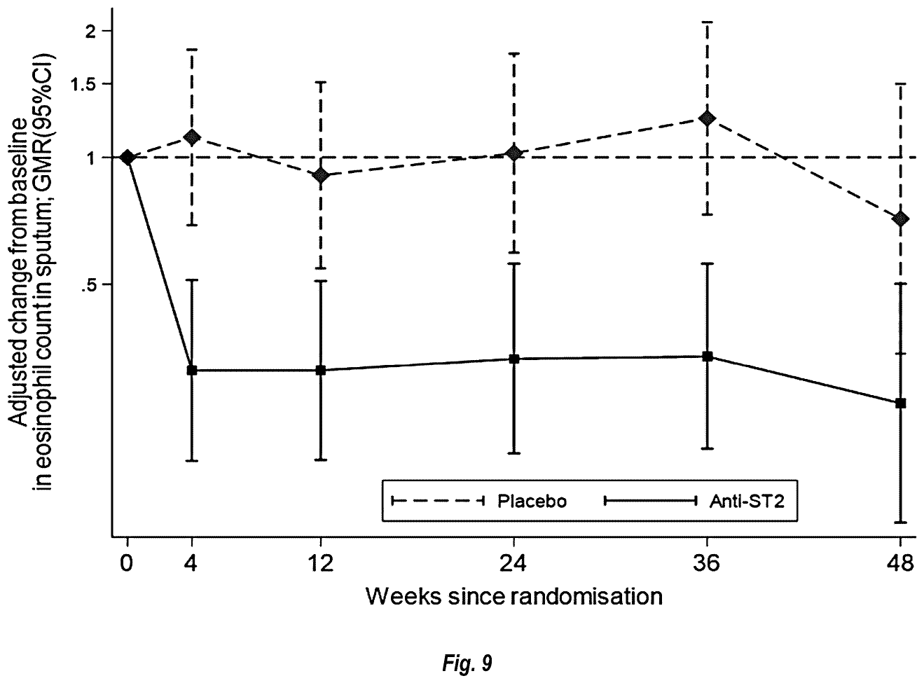

FIG. 9 shows the change from baseline in % sputum eosinophil count over 48 weeks.

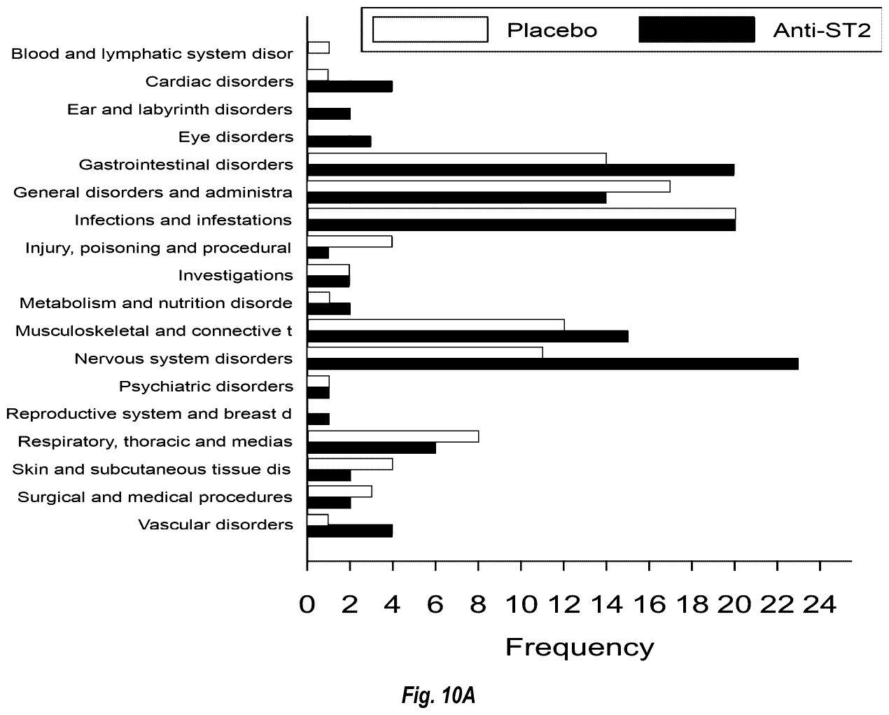

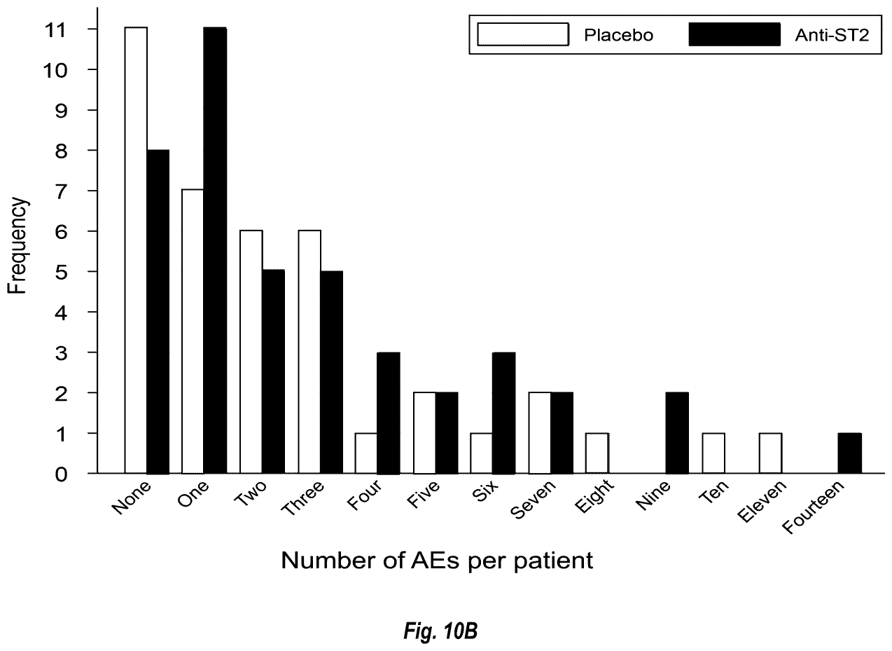

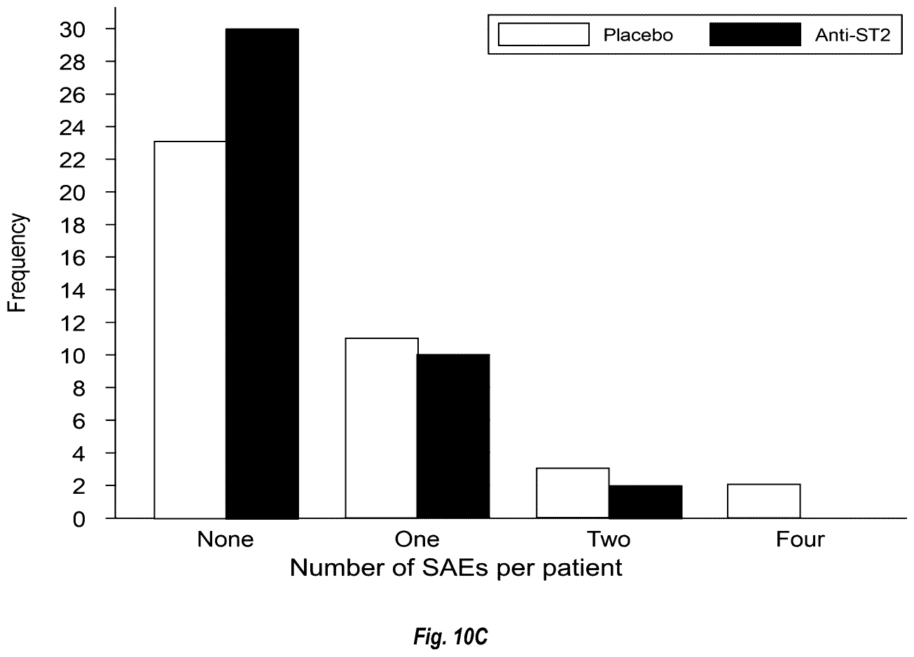

FIG. 10A-10C shows certain adverse events for treatment compared to placebo. FIG. 10A provides the frequency of certain adverse events (AEs), while FIG. 10B-10C provides number of adverse events or number of serious adverse events (SAEs) per patient.

In summary, numerical reductions of annualized exacerbation rate were observed in this study, as well as statistically significant improvement in SGRQ. There was also a trend in improvement of FEV1. The reduction of blood eosinophils seen as early as week 4 provides a pharmacodynamic biomarker. The incidence of treatment-emergent adverse events and serious adverse events were similar between groups and did not alter the safety profile of astegolimab from other studies.

### Example 2: A Randomized, Double-Blind, Placebo-Controlled, Multicenter Study to Evaluate the Safety and Efficacy of Astegolimab in Patients with Chronic Obstructive Pulmonary Disease

This is a Phase II, randomized, double-blind, placebo-controlled, multicenter study to evaluate the efficacy, safety and pharmacokinetics of astegolimab in combination with standard of care compared with placebo in combination with SOC, in patients with COPD who are former or current smokers and have a history of frequent exacerbations. Approximately 930 patients who meet the entry criteria in centers will be treated. Specific objectives and corresponding endpoints for the study are outlined below.

### Primary Efficacy Objective

The primary efficacy objective for this study is to evaluate the efficacy of astegolimab compared with placebo on the basis of the following endpoint:
Annualized rate of moderate and severe COPD exacerbations over the 52-week treatment period
   A moderate COPD exacerbation is defined as new or increased COPD symptoms (e.g., dyspnea, sputum volume, and sputum purulence) that lead to treatment (duration ≥3 days) with systemic corticosteroids (oral, IV, or intramuscular [IM]) at a dose of >10 mg/day prednisolone equivalent and/or antibiotics.
   A severe COPD exacerbation is defined as new or increased COPD symptoms that lead to hospitalization (duration >24 hours) or lead to death.

### Secondary Efficacy Objective

The secondary efficacy objective for this study is to evaluate the efficacy of astegolimab compared with placebo on the basis of the following endpoints:
Time to first moderate or severe COPD exacerbation during the 52-week treatment Period
Absolute change from baseline in health-related quality of life (HRQoL) at Week 52, as assessed through the St. George's Respiratory Questionnaire-COPD (SGRQ-C) total score
Proportion of patients with improvement in HRQoL, defined as a decrease from baseline of ≥4 points in SGRQ-C total score, at Week 52
Absolute change from baseline in post-bronchodilator FEV1 (liters) at Week 52
Absolute change from baseline in Evaluating Respiratory Symptoms in COPD (E-RS®:COPD) total score at Week 52
Annualized rate of severe COPD exacerbations over the 52-week treatment period
Absolute change from baseline in five-repetition sit-to-stand test (5STS) time (seconds) at Week 52

### Additional Efficacy Objective

The further efficacy objective for this study is to evaluate the efficacy of astegolimab compared with placebo on the basis of the following endpoints:
Annualized rate of EXAcerbations of Chronic Pulmonary Disease Tool (EXACT@)-defined exacerbation events over the 52-week treatment period
   An EXACT exacerbation event is defined as an EXACT total score of ≥12 points sustained for 2 days or ≥9 points sustained for 3 days.
Proportion of patients with HRQoL improvement, defined as a decrease from baseline of ≥4 points in SGRQ-C total score, at Week 12 and Week 24
Proportion of patients with symptom improvement, defined as a decrease of ≥2 points from baseline in E-RS:COPD total score, at Week 24 and Week 52
Absolute change from baseline in post-bronchodilator FEV1 (liters) at Weeks 12, 24, and 36
Absolute change from baseline in 5STS time (seconds) at Week 24
Annualized rate of moderate COPD exacerbations over the 52-week treatment period
Duration of hospital stay for severe COPD exacerbations
Proportion of severe COPD exacerbations requiring hospital readmission within 30 days
Absolute change from baseline in residual volume/total lung capacity ratio at Week 52
Absolute change from baseline in daily step count at Weeks 12, 24, and 52
Absolute change from baseline in time in moderate and vigorous physical activity at Weeks 12, 24, and 52
Absolute change from baseline in COPD Assessment Test™ (CAT™) score at Week 52
Annualized rate of moderate and severe COPD exacerbations over the blinded treatment period
Absolute change from baseline in short-acting rescue inhaler use
Absolute change in nightly total sleep time, as measured by actigraphy

### Safety Objective

The safety objective for this study is to evaluate the safety of astegolimab compared with placebo on the basis of the following endpoints:
Incidence and severity of adverse events, with severity determined according to the Division of AIDS Table for Grading the Severity of Adult and Pediatric Adverse Events, Version 2.1 (DAIDS Table v2.1) toxicity scale
Change from baseline in targeted vital signs
Change from baseline in targeted clinical laboratory test results and ECGs

### Pharmacokinetic Objectives

The pharmacokinetic (PK) objective for this study is to characterize the astegolimab PK profile on the basis of the following endpoint:
Serum concentration of astegolimab at specified timepoints The exploratory PK objectives for this study are as follows:
To evaluate potential relationships between drug exposure and the efficacy and safety of astegolimab on the basis of the following endpoints:
   Relationship between serum concentration or PK parameters for astegolimab and efficacy endpoints
   Relationship between serum concentration or PK parameters for astegolimab and safety endpoints
To evaluate potential relationships between selected covariates and exposure to astegolimab on the basis of the following endpoint:
   Relationship between selected covariates and serum concentration or PK parameters for astegolimab

### Immunogenicity Objectives

The immunogenicity objective for this study is to evaluate the immune response to astegolimab on the basis of the following endpoint:
Prevalence of anti-drug antibodies (ADAs) at baseline and incidence of ADAs during the study

The exploratory immunogenicity objective for this study is to evaluate potential effects of ADAs on the basis of the following endpoint:
Relationship between ADA status and efficacy, safety, or PK endpoints

### Biomarker Objective

The exploratory biomarker objective for this study is to identify and/or evaluate biomarkers that are predictive of response to astegolimab (i.e., predictive biomarkers), are associated with progression to a more severe disease state (i.e., prognostic biomarkers), can provide evidence of astegolimab activity (i.e., pharmacodynamic biomarkers), or can increase the knowledge and understanding of disease biology and drug safety, on the basis of the following endpoint:
Relationship between biomarkers in blood, plasma, serum, and sputum and Nasosorption™ samples and efficacy, safety, PK, immunogenicity, or other biomarker endpoints

Exploratory biomarkers include, but are not limited to, the analysis of eosinophils, IL-33 pathway markers (e.g., sST2), inflammatory proteins (e.g., fibrinogen, C-reactive protein), and single nucleotide polymorphisms of selected genes (e.g., IL1RL1, IL33 and other genes related to COPD).

### Interleukin-33 and ST2

Astegolimab (also known as MSTT1041A or Ab2) is a fully human, IgG2 monoclonal antibody that binds with high affinity to the IL-33 receptor, ST2, thereby blocking the signaling of interleukin-33 (IL-33), an inflammatory cytokine of the interleukin-1 (IL-1) family and member of the “alarmin” class of molecules. Astegolimab has subnanomolar affinity and potency, is active in blood, and lacks agonistic activity.

IL-33 is considered an “alarmin” or a damage-associated molecular pattern molecule that is constitutively expressed on epithelial cells and released upon cell injury or stress from exposure to such exogenous stimuli as allergens, toxins, or infections. IL-33 is a member of the IL-1 family of cytokines (Sims and Smith 2010) with potential as a target in the treatment of asthma, COPD, and atopic dermatitis.

High levels of IL-33 are found in stromal cells, particularly at barrier surfaces such as the lung and gastrointestinal tract. Within the lung, IL-33 is detected in multiple cell types, including epithelial cells, endothelial cells, and fibroblasts (Liew et al. 2016). IL-33 bioavailability is tightly regulated, and under homeostatic conditions, this protein is sequestered in the nuclei of these cells. Cellular damage caused by injury, mechanical stress, or death leads to the release of bioactive IL-33 into circulation, where it initiates and propagates innate and adaptive immune responses. The receptor for IL-33, ST2, is expressed on multiple cell types implicated in pulmonary inflammation and disease, including mast cells, eosinophils, basophils, innate lymphoid cells, T lymphocytes, macrophages, and endothelial cells.

IL-33 is also linked to the function of type-2 innate lymphocytes that accumulate in the lung and promote T helper type 2 (Th2) cell inflammation, even in the absence of antigen stimulation (Scanlon and McKenzie 2012). In some settings, IL-33 will also promote type 1 responses, such as interferon (IFN)-γ production from natural killer (NK) cells or NK T cells. As such, IL-33 may be involved in multiple inflammatory pathways implicated in COPD.

IL-33 activates these various immune cells through its receptor ST2, also known as Il-1 receptor-like 1 (IL-1RL1) (Nabe 2014). The binding of IL-33 to ST2 promotes association with the shared IL-1 family subunit, IL-1RAcP, to form the active IL-33 receptor. Intracellular signaling induced by IL-33 promotes expression of inflammatory genes. A secreted soluble form of ST2 (sST2) arises from alternative splicing, is elevated in settings of inflammation, and acts as a decoy to bind and inhibit released IL-33 (Hayakawa et al. 2007).

Excess extracellular IL-33 is highly inflammatory in pulmonary tissue and is capable of triggering local inflammation that can lead to airway hyperresponsiveness (AHR) and mucus production, important components of exacerbations. Airway administration of IL-33 in mice leads to an infiltration of inflammatory cells in bronchoalveolar lavage fluid, including eosinophils and neutrophils, as well as elevated interleukin-5, interleukin-13, eotaxin, and thymus and activation-regulated chemokine (also known as TARC/CCL17) (Louten et al. 2011). Given the diverse nature of signals that lead to IL-33 release and the wide range of target cells, IL-33 is implicated in a number of pathological pathways. IL-33 release can trigger acute exacerbations and/or disease progression in asthma, COPD, idiopathic pulmonary fibrosis, and acute respiratory distress syndrome. IL-33 activity is elevated following viral infections, and inhibition of this pathway reduces virus-induced exacerbations in rodent models of asthma and COPD (Werder et al. 2018; Ravanetti et al. 2019). ST2- or IL-33-deficient mice exposed to cigarette smoke have decreased inflammatory responses in response to subsequent respiratory viral infections, without compromising anti-viral host defense (Kearley et al. 2015). The absence of the IL-33 pathway significantly reduced viral-induced leukocyte migration into the lung, inflammatory cytokine expression, and subsequent pulmonary pathology. For these reasons, inhibiting ST2 is hypothesized to confer clinical benefit to patients with COPD by limiting excessive inflammatory sequelae.

### Overview of Study Design

This is a Phase IIb, randomized, double-blind, placebo-controlled, multicenter study to evaluate the efficacy, safety and pharmacokinetics of astegolimab in combination with standard of care (SOC) compared with placebo in combination with SOC, in patients with COPD who are former or current smokers and have a history of frequent exacerbations. Approximately 930 patients with COPD are expected to be enrolled globally.

Following a screening period of at least 7 days to up to 4 weeks, patients will be randomized in a 1:1:1 ratio to one of three treatment arms to receive blinded treatment with either astegolimab or placebo. Randomization will be stratified by smoking status at screening (former smoker vs. current smoker) and region.

The first dose of study drug (astegolimab or placebo) will be administered on Day 1; treatment will continue through at least Week 50, followed by a 12-week safety follow-up period. The treatment regimens for each arm are as follows:
Astegolimab 476 mg SC every 2 weeks (Q2W)
Astegolimab 476 mg SC every 4 weeks (Q4W)
   To ensure that all study patients undergo the same visit schedule, patients randomized to the Q4W dosing arm will alternate between injections of astegolimab and placebo every 2 weeks (beginning with astegolimab on Day 1), thus receiving astegolimab every 4 weeks.
Placebo SC Q2W

Patients will return to the clinic every 2 weeks through the treatment completion visit at Week 52 (or the end of an additional treatment period, as described below, if applicable). The primary endpoint analyses will be conducted using the 52-week treatment period data for all patients.

### Formulation

Advantageously, 476 mg of astegolimab can be administered via a single pre-filled syringe for subcutaneous administration. Astegolimab and placebo will be supplied as a sterile liquid in 2.25 mL pre-filled syringes with a needle safety device, providing 238 mg/1.7 mL of astegolimab or placebo.

### Target Product Profile

The population is adult patients with COPD and a history of ≥2 exacerbations despite treatment with ICS therapy plus LABA and/or LAMA for ≥12 months prior to visit 1. COPD is defined as FEV1/FVC ratio <0.7, bronchodilator response <12%. FEV1 20-80%; current and former smokers with ≥10 pack-years of tobacco use, ≥1 exacerbation in the previous year, optimized on standard-of-care therapy to include ≥2 long-acting inhalers.

The primary endpoint of annual exacerbation (moderate and severe) rate reduction (AERR) at 52 weeks is >25% in all-comers, >35% in all-comers, or >45% in all-comers.

A secondary endpoint is improvement in health-related quality of life as measured by the SGRQ of 4 points.

A secondary endpoint is 5% improvement in FEV1.

### Rationale for Patient Population

Frequent exacerbations significantly impact symptoms, health-related quality of life, physical function, disease progression, healthcare utilization, and mortality in COPD (Anzueto 2010). Patients with COPD who have two or more moderate to severe exacerbations within a 12-month period are at the greatest continued risk of future exacerbations (Han 2017). Therefore, it is in this group of patients that the most benefit is expected from a reduction in exacerbations. History of prior exacerbations is a stronger predictor of future exacerbation risk than severity of airflow obstruction, inflammatory markers, and functional or clinical markers (Hurst 2010). While the frequency of exacerbations increases with severity of airflow obstruction, a significant proportion of patients with moderate airflow obstruction experience a high frequency of exacerbations (Decramer 2009). This study will therefore enroll patients with moderate to very severe airflow obstruction who have a history of frequent exacerbations (defined as an annual rate of two or more exacerbations).

### Rationale for Control Group

The study will compare the efficacy, safety and pharmacokinetics of astegolimab in combination with SOC compared with placebo in combination with SOC in patients with COPD. Treatment with SOC ensures that all patients receive therapy for COPD; the placebo control group takes into account safety, efficacy, and ethical considerations for studying the effects of astegolimab.

### Rationale for Biomarker Assessments

COPD is a heterogeneous disease, and IL33 and sST2 expression has been shown to vary among patients. Therefore, all patients may not be equally likely to benefit from treatment with astegolimab. Biomarker assessments, before and at various timepoints after treatment, will be used to provide evidence of the biologic activity of astegolimab in patients, identify biomarkers that may be predictive of response to astegolimab, define PK and/or PD relationships, advance the understanding of the mechanism of action of astegolimab in patients, support selection of a recommended dose regimen, and increase the knowledge and understanding of disease biology. Exploratory biomarker analysis may include, but will not be limited to, analysis of eosinophils, IL-33 pathway markers (e.g., sST2), and inflammatory mediators (e.g., fibrinogen and C-reactive protein). For example, patients may be stratified by eosinophil count, such as a baseline blood eosinophil count <300 eosinophils/μL, a baseline blood eosinophil count ≤170 eosinophils/μL, or a baseline blood eosinophil count ≤150 eosinophils/μL.

A blood sample will be collected for DNA extraction to enable identification of specific germline mutations in IL1RL and IL33 and other genes related to COPD that may be predictive of response to study drug, are associated with disease progression, or can increase the knowledge and understanding of disease biology.

Exploratory research on safety biomarkers may be conducted to support future drug development. Research may include further characterization of a safety biomarker or identification of safety biomarkers that are associated with susceptibility to developing adverse events or can lead to improved adverse event monitoring or investigation. Adverse event reports will not be derived from safety biomarker data by the Sponsor, and safety biomarker data will not be included in the formal safety analyses for this study. In addition, safety biomarker data will not inform decisions on patient management.

### Patients

Approximately 930 patients with COPD and a history of frequent exacerbations will be enrolled in this study.

### Inclusion Criteria

Patients must meet the following criteria for study entry:
Signed Informed Consent Form
Age 40-90 years at time of signing Informed Consent Form
Ability to comply with the study protocol
Documented physician diagnosis of COPD made at least 12 months prior to screening
History of frequent exacerbations, defined as having had two or more moderate or severe exacerbations occurring within a 12-month period in the 24 months prior to screening
   Exacerbations should have been treated with systemic corticosteroids and/or antibiotics.
   A moderate COPD exacerbation is defined as new or increased COPD symptoms (e.g., dyspnea, sputum volume, and sputum purulence) that lead to treatment (duration ≥3 days) with systemic corticosteroids (oral, IV, or IM) at a dose of >10 mg/day prednisolone equivalent and/or antibiotics. Prior use of antibiotics alone does not qualify as a moderate exacerbation, unless the use was specifically for the treatment of worsening symptoms of COPD.
   A severe COPD exacerbation is defined as new or increased COPD symptoms that lead to hospitalization (duration >24 hours) or lead to death.
Post-bronchodilator FEV1 ≥20 and <80% of predicted normal value at screening, as verified by over-reader
Post-bronchodilator FEV1/FVC<0.70 at screening, as verified by over-reader
mMRC score ≥2 at screening
Ability to perform 5STS within 60 seconds at screening
Current smoker or former smoker with a minimum of 10 pack-year history (e.g., 20 cigarettes/day for 10 years)
   A former smoker is defined as meeting the criteria above but has not used inhaled tobacco products or inhaled marijuana within 6 months prior to screening, through use of cigarettes, cigars, electronic cigarettes, vaporizing devices, or pipe.
   Note that at screening, patients who meet the protocol definition of current smoker will receive smoking cessation counseling.
History of one of the following combinations of optimized, stable, standard of care COPD maintenance therapy for at least 4 weeks prior to screening, with no anticipated changes in therapy prior to initiation of study drug and throughout the study:
   Inhaled corticosteroid (ICS) ≥500 mcg/day fluticasone propionate dose-equivalent plus long-acting beta-agonist (LABA)
   Long-acting muscarinic antagonist (LAMA) plus LABA
   ICS≥500 mcg/day fluticasone propionate dose-equivalent plus LAMA plus LABA
Demonstrated ability to use and comply with electronic diary (eDiary) requirements, defined as completion of all questions on at least 5 out of 7 consecutive days within the 14 days after the screening visit
   Patients unable to demonstrate compliance with the eDiary within the first 2 weeks of screening will be screen failed. Patients will have the opportunity to demonstrate eDiary compliance if re-screened.
For women of childbearing potential: agreement to remain abstinent (refrain from heterosexual intercourse) or use contraception, as defined below:
   Women must remain abstinent or use contraceptive methods with a failure rate of <1% per year during the treatment period and for 12 weeks after the final dose of astegolimab.
   A woman is considered to be of childbearing potential if she is postmenarchal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices. The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.
For men: agreement to remain abstinent (refrain from heterosexual intercourse) or use a condom, and agreement to refrain from donating sperm, as defined below:
   With a female partner of childbearing potential or pregnant female partner, men must remain abstinent or use a condom during the treatment period and for 12 weeks after the final dose of astegolimab to avoid exposing the embryo. Men must refrain from donating sperm during this same period.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of preventing drug exposure. If required per local guidelines or regulations, information about the reliability of abstinence will be described in the local Informed Consent Form.
For patients enrolling in the Airway Biomarker Substudy: ability to provide at least 1 mL of induced sputum at screening

### Exclusion Criteria

Patients who meet any of the following criteria will be excluded from study entry:
Pregnant or breastfeeding, or intending to become pregnant during the study or within 12 weeks after the final dose of study drug.
   Women of childbearing potential must have a negative serum pregnancy test result at screening and a negative urine pregnancy test on Day 1 prior to initiation of study drug.
Current documented diagnosis of asthma according to the Global Initiative for Asthma guidelines or other accepted guidelines within 5 years prior to screening
History of clinically significant pulmonary disease other than COPD (e.g., pulmonary fibrosis, sarcoidosis, chronic pulmonary embolism or primary pulmonary hypertension, alpha-1-antitrypsin deficiency)
Clinically significant abnormalities requiring clinical follow-up as indicated by chest X-ray or chest CT scan performed within 6 months prior to screening
   Chest X-ray must be performed at screening if results from a chest X-ray or chest CT scan performed within 6 months prior to screening are not available.
Presence of risk factors for aspiration pneumonia (e.g., neurologic disease such as uncontrolled epilepsy) in the opinion of the investigator
History of long-term treatment with oxygen at >4.0 liters/minute
   While breathing supplemental oxygen, patient should demonstrate an oxyhemoglobin saturation of ≥89%.
History of a severe allergic reaction or anaphylactic reaction to a biologic agent or known hypersensitivity to any component of the study drug
Lung volume reduction surgery or procedure within 12 months prior to screening
Participation in or planned participation in a new pulmonary rehabilitation program within 4 weeks prior to screening and throughout the study treatment period
   Patients who are in the maintenance phase of a rehabilitation program are eligible.
History of lung transplant
Occurrence of moderate or severe COPD exacerbation, COVID-19, upper or lower respiratory infection, pneumonia, or hospitalization of ≥24 hours duration within 4 weeks prior to initiation of study drug
Any prior treatment with astegolimab
Treatment with oral, IV, or IM corticosteroids (>10 mg/day prednisolone equivalent) within 4 weeks prior to initiation of study drug
Treatment with investigational therapy within 3 months or 5 drug-elimination half-lives (whichever is longer) prior to screening
Treatment with a licensed biologic agent (e.g., omalizumab, dupilumab, and/or anti-IL-5 therapies) within 3 months or 5 drug-elimination half-lives (whichever is longer) prior to screening
Initiation of a methylxanthine preparation, maintenance macrolide therapy, and/or PDE4 inhibitor within 4 weeks prior to screening
Initiation of or change in non-biologic immunomodulatory or immunosuppressive therapy within 3 months prior to screening
Treatment that is considered palliative (e.g., for life expectancy <12 months)
Use of any of the following treatments within 4 weeks prior to screening, or any condition that, in the opinion of the investigator, is likely to require such treatment during the course of the study, unless deemed acceptable in consultation with the Medical Monitor:
   Treatment with immunoglobulin or blood products.
   Treatment with any live or attenuated vaccine (including any approved, live SARS-CoV-2 vaccine) within 4 weeks prior to screening or during the screening period, or anticipated need for live, attenuated vaccine during the course of the study, unless the vaccine is deemed medically necessary and no inactivated vaccine alternatives are available.
Administration of non-live SARS-CoV-2 vaccine (with full marketing authorization or temporary), including those delivered by non-replicating viral vectors, within 7 days prior to screening
Planned surgical intervention during the study
Positive hepatitis C virus (HCV) antibody test result accompanied by a positive HCV RNA test at screening
Unacceptable test results for hepatitis B surface antigen (HBsAg), hepatitis B surface antibody (HBsAb), and total hepatitis B core antibody (HBcAb) at screening, defined as meeting either of the following criteria:
   Positive HBsAg test at screening.
   Negative HBsAg test at screening, with negative HBsAb test accompanied by positive total HBcAb test, followed by quantitative hepatitis B virus (HBV) DNA ≥20 IU/mL. Inability to perform HBV DNA test is exclusionary.
   Patients with a negative HBsAg test and positive HBsAb test are eligible.
Known immunodeficiency including, but not limited to, HIV infection
Known evidence of active or untreated latent tuberculosis
Substance abuse, as determined by the investigator, within 12 months prior to screening
History of malignancy within 5 years prior to screening, with the exception of malignancies with a negligible risk of metastasis or death (e.g., 5-year overall survival rate >90%), such as adequately treated carcinoma in situ of the cervix, non-melanoma skin carcinoma, localized prostate cancer, or ductal carcinoma in situ
Any other serious medical condition or abnormality in clinical laboratory tests that, in the investigator's judgment, precludes the patient's safe participation in and completion of the study
Unstable cardiac disease, myocardial infarction, or New York Heart Association Class III or IV heart failure within 12 months prior to screening
History or presence (as verified by over-reader) of an abnormal ECG that is deemed clinically significant by the investigator, including complete left bundle branch block or second- or third-degree atrioventricular heart block
QT interval corrected through use of Fridericia's formula (QTcF) (as verified by over-reader) >450 ms if patient is male or QTcF>470 ms if patient is female
   For male or female patients with QRS>120: QTcF>480 ms.
History of ventricular dysrhythmias or risk factors for ventricular dysrhythmias such as structural heart disease (e.g., severe left ventricular systolic dysfunction, significant left ventricular hypertrophy with strain), or family history of sudden unexplained death or long QT syndrome

### Standard of Care COPD Maintenance Therapy

All patients must be on one of the following combinations of optimized, stable, standard of care COPD maintenance therapy for at least 4 weeks prior to screening, with no anticipated changes in therapy prior to initiation of study drug and throughout the study:
ICS≥500 mcg/day fluticasone propionate dose-equivalent plus long-acting betaagonist (LABA)
Long-acting muscarinic antagonist (LAMA) plus LABA
ICS≥500 mcg/day fluticasone propionate dose-equivalent plus LAMA plus LABA

At screening, patients will be instructed on the proper technique recommended for administration of their ICS and inhaled bronchodilator therapy. From 4 weeks prior to screening through completion of the study, the doses of background COPD medications should remain stable. If changes to the background COPD medications are unavoidable, the patient may be switched to another brand or formulation that is equivalent to the medication that the patient was receiving at the time of study entry. Proposed changes should be discussed with the Medical Monitor. All changes to a patient's background medications should be documented in the Concomitant Medications eCRF.

### COPD Exacerbation Assessment

At each study visit, an assessment will be conducted to determine if the patient experienced any protocol-defined acute COPD exacerbation since the previous study visit.

An acute COPD exacerbation is defined as an exacerbation that meets the criteria for either moderate or severe exacerbation as follows:
A moderate COPD exacerbation is defined as new or increased COPD symptoms (e.g., dyspnea, sputum volume, and sputum purulence) that lead to treatment (duration ≥3 days) with systemic corticosteroids (oral, IV, or IM) at a dose of >10 mg/day prednisolone equivalent and/or antibiotics.
A severe COPD exacerbation is defined as new or increased COPD symptoms that lead to hospitalization (duration >24 hours) or lead to death.

An acute COPD exacerbation occurring ≤7 days after the final dose of systemic corticosteroids (oral, IM, or IV) and/or antibiotic therapy indicated for a previous exacerbation will be captured as a single exacerbation event.

Given that the annualized rate of COPD exacerbations is the primary endpoint in this study, a dedicated eCRF will be used to record information regarding protocol-defined acute exacerbation events. An acute COPD exacerbation must also be reported as an adverse event (or serious adverse event as applicable). Sites should record all medications used for treatment of any COPD exacerbation in the appropriate eCRF.

### Results and Conclusions

It is anticipated that the treatment herein with Ab2 will achieve any one or more of the primary, secondary, or additional endpoints, while having acceptable toxicity according to the safety endpoints specified herein.

### Example 3: Prognostic and Predictive Biomarkers for COPD Exacerbations and Treatment Response

Post-hoc analysis of ST2OP study (Example 1) was conducted to assess the prognostic and predictive performance of certain biomarkers for COPD.

### Genotyping and Sample-Level Genetic Estimates

Response analysis in subgroups determined by SNPs for alleles associated with the IL33/ST2 axis was conducted. Known amino acid changing SNPs that have an impact on function (Ramirez-Carrozzi V JACI 2014) were considered for association to patient response to aST2. Determination of the genotype of a patient involve procedures well known in the field of molecular genetics. Here, patient samples, collected prior to administration of ST2 antagonist, were genotyped on the Global Screening Array (GSA) and additional variants were imputed using BEAGLE v5.0 (Browning et al., Am J Hum Genet, 2018) with 1000 Genomes reference haplotypes (The 1000 Genomes Project Consortium Nature, 2015).

Sample relatedness was estimated using PLINK v1.90b3.42 (Chang et al GigaScience, 2015) with the “--genome” argument of the plink function. Genetic ancestry estimates per individual were derived using ADMIXTURE v1.3.0 (Alexander et al BMC Bioinformatics, 2011). Principal components (PCs) for samples with fraction European ancestry >0.7 were estimated using EIGENSOFT v 6.1.4 (Price et al Nature Genetics, 2006). PCs were inferred on a subset of unrelated individuals, while PCs for remaining related individuals were estimated by projecting genetic data onto the inferred PCs.

ST2OP pharmacogenetic analysis of functional IL1RL1 TIR domain tagging SNPs (Ramirez-Carrozzi V. JACI 2014) was conducted by mixed effect negative binomial regression of exacerbation counts, including genotype, study stratification terms (treatment arm and history of exacerbations), PC1, PC2, and 1st degree family membership as a random effect. The time at risk was included as an offset term to account for variability of the placebo controlled period for study participants. Only subjects who were among the Intent To Treat (ITT) population and were of majority European ancestry (fraction European ancestry estimated by ADMIXTURE >0.7) were included in this analysis. The placebo corrected treatment effect was estimated by linear contrasts extracted from the regression model.

Serum Soluble ST2 (sST2)

Soluble ST2 (sST2) was measured in serum using an ELISA from R&D Systems (Catalog No. DST200, Quantikine).

The predictive biomarker effect of soluble ST2 (sST2) was conducted by negative binomial regression of exacerbation counts, including screening visit sST2 status (< or > screening visit sST2 median), study stratification terms (treatment arm and history of exacerbations) and gender. The time at risk was included as an offset term to account for variability of the placebo controlled period for study participants. Only subjects who were among the Intent To Treat (ITT) population were included in this analysis. The placebo corrected treatment effect was estimated by linear contrasts extracted from the regression model.

ZENYATTA Soluble ST2 Predictive Biomarker Analyses The predictive biomarker effect of soluble ST2 (sST2) was conducted by negative binomial regression of exacerbation counts, including screening visit sST2 status (< or > screening visit sST2 median), study stratification terms (treatment arm, history of exacerbations, ICS dose, and region) and gender. The time at risk was included as an offset term to account for variability of the placebo controlled period for study participants. Only subjects who were among the Intent To Treat (ITT) population were included in this analysis. The placebo corrected treatment effect was estimated by linear contrasts extracted from the regression model.

### Alpha-Diversity Microbiome

Alpha-diversity, or α-diversity, is a measure of ecological diversity and can be employed for estimating microbiome diversity in a particular sample using high dimensional microbiome assays, e.g. sequencing. Sputum 16s rRNA v4 amplicon sequencing was employed for microbiome analysis and negative binomial regression including study stratification factors and baseline α-diversity dichotomized at the observed median as model terms to estimate treatment effect and annualized exacerbation rate. Model estimates are represented as lsmeans [5% confidence intervals]. Shannon-Weaver method was used to calculate the α-diversity index (Hurlbert, S. H. Ecology 1971).

### Statistics

Statistical analysis was conducted using the statistical programming environment R (available at r-project.org). 95% confidence intervals are presented for all analyses.

### Results and Discussion

### Single Nucleotide Polymorphisms (SNPs) Tagging IL1RL1 TIR Domain Functional Variants are Predictive of Response to Targeted Therapies for IL-33 Mediated Disorders.

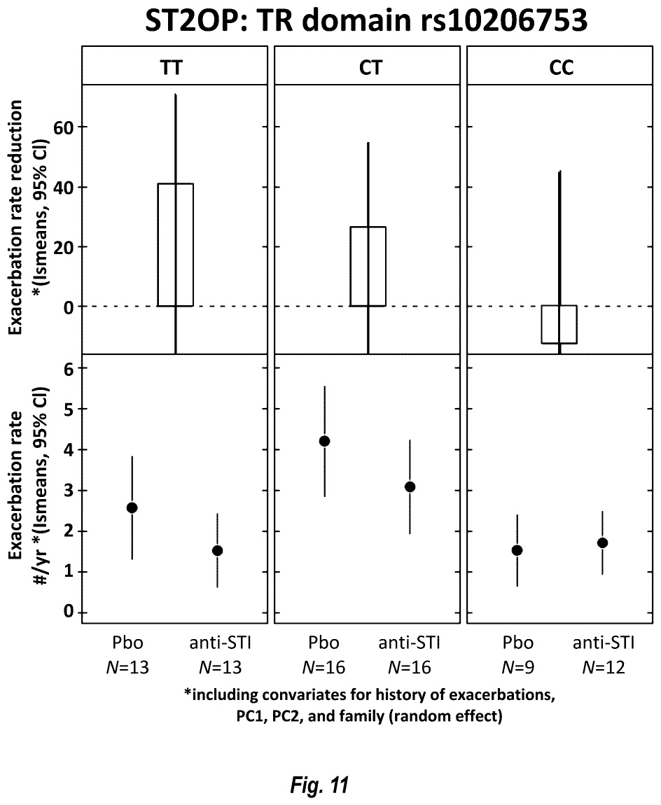

Toll/IL-1R (TIR) domain functional variants have been previously described as impacting IL-33 signaling strength and are in linkage disequilibrium (LD) with asthma risk loci (Ramirez-Carrozzi, 2014). These functional variants which are in LD could identify patients with enhanced IL-33 mediated disease and therefore may benefit from IL-33/ST2 pathway inhibition. The pharmacogenetic effect ofILIRLI TIR domain functional variants were assessed in a placebo controlled interventional study of COPD patients treated with anti-ST2 (astegolimab) (ST2OP, Example 1) for the primary outcome of the study. Polymorphism rs10206753 (SEQ ID NO: 41) is in linkage disequilibrium with common, functional IL1RL1 TIR domain variants (Ramirez-Carrozzi, 2014) and was utilized as a tag SNP for this haplotype. Consistent with our hypothesis, homozygous carriers of the alternate allele (CC), associated with diminished IL-33 signaling, derived the least efficacy (−12.2 [−8.0, 29.8] %); in contrast, homozygous carriers of the common allele (TT), associated with enhanced IL-33 signaling, derived maximal clinical benefit among genotypes (FIG. 11) (41.1 [6.4, 62.9]%). Heterozygous carriers (CT) derived and intermediate degree of efficacy (26.6 [−19.1, 54.8]%). Taken together, these data suggest that this is an additive model of a pharmacogenetic effect.

### Peripheral Blood Soluble ST2 Levels are Predictive of Response to Targeted Therapies for IL-33 Mediated Disorders.

The receptor for IL-33, ST2-L and the decoy soluble ST2 (sST2) are expressed by IL1RL1 and their expression is determined by alternative promoter usage and splicing. sST2 expression can be induced by IL-33 signaling as well as other mediators activating the NFκB and MAPKK signaling pathways, therefore sST2 levels may be a biomarker of activation of that pathway (Ho JE J CI 2013). We hypothesized that serum levels of sST2 may reflect extent of IL-33 mediated disease and therefore may predict response to IL-33/ST2 pathway inhibitors. To test this, the predictive effect of pre-treatment serum sST2 levels in a placebo controlled interventional study of asthma (ZENYATTA, described elsewhere) and COPD (ST2OP, Example 1) patients treated with anti-ST2 (astegolimab) for the primary outcome of the respective studies was assessed. The patient population was categorized on the basis of having low or high levels of sST2 (< or >median). The median sST2 level at baseline was 19.1 ng/mL.

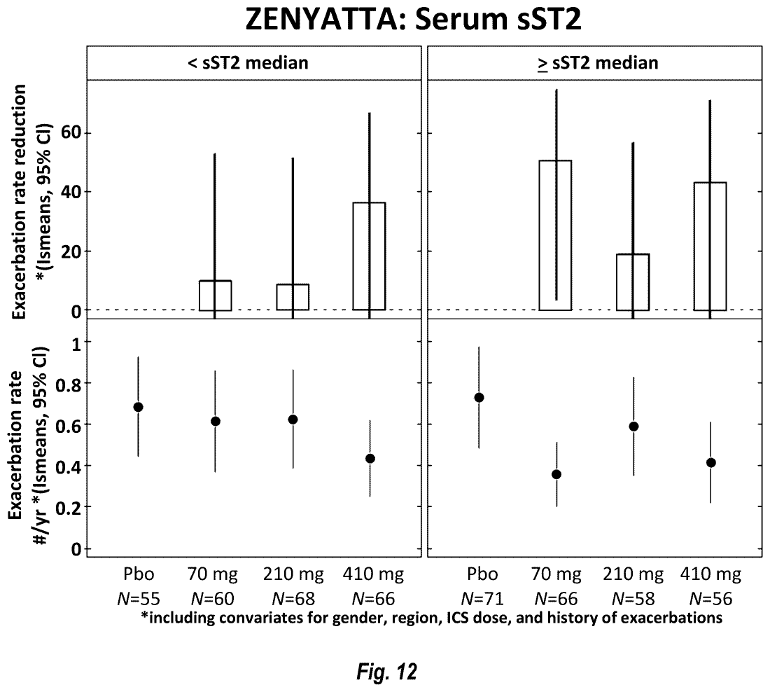

Consistent with the hypothesis, in ZENYATTA (FIG. 12), subjects who had high levels of serum sST2 (>median) derived enhanced treatment benefit with anti-ST2 (70 mg 51%, 210 mg 19%, 490 mg 43%) as compared to subjects with low levels (<median) (70 mg 10%, 210 mg 9%, 490 mg 36%).

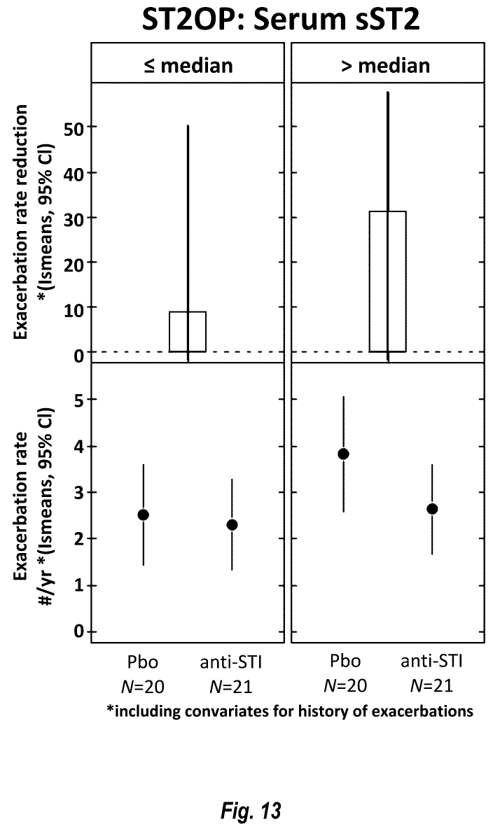

Confirming the observation in ZENYATTA, ST2OP participants having high levels of serum sST2 (>median) derived enhanced treatment benefit with anti-ST2 (31.1 [−12.3, 57.7]%) as compared to subjects with low levels (<median) (FIG. 13) (8.6 [−67.5, 50.1]%).

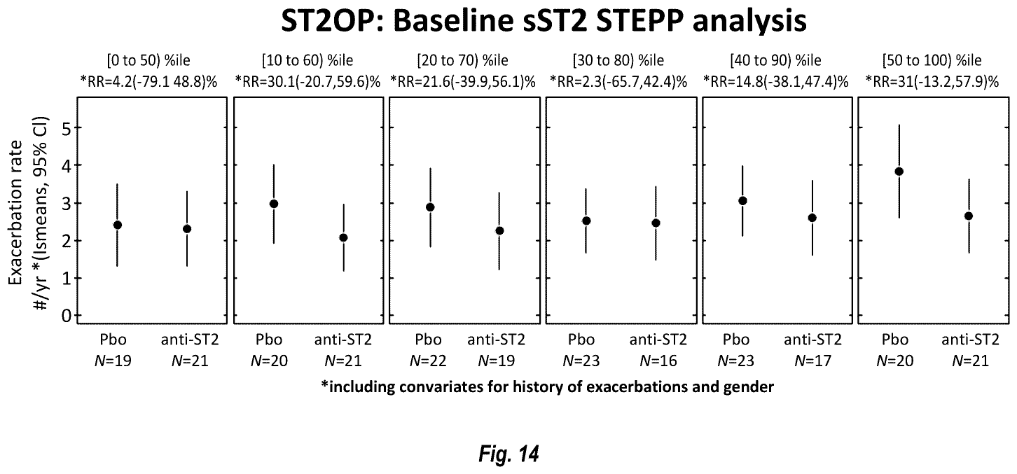

As serum sST2 is a continuous as opposed to a categorical biomarker without any established reference ranges, STEPP analysis (Lazar A A, J. Clin. Oncol. 2010 Oct. 10; 28(29):4539-44) was performed to better understand the relationship between pre-treatment sST2 levels and treatment effect (FIG. 14). STEPP analysis supports that increasing range of sST2 levels is associated with greater treatment effect and prognostic for exacerbations for placebo treated subjects.

### Airway Microbiome Diversity is Prognostic for COPD Exacerbations and Treatment Response to Anti-ST2 Therapy.

COPD exacerbations are heterogenous with respect to the airway host inflammatory phenotype and associated microbial profiles. Exacerbations marked by elevated airway innate cytokine levels, i.e. IL-1β and TNFα, are associated with bacterial infection, neutrophilic inflammation and lung microbial dysbiosis (Ghebre M A JACI 2018). As this exacerbation subtype is preceded by lung microbial dysbiosis during stable disease (Chakrabarti A ERJ OR 2021), we hypothesized that lung microbiome α-diversity would predict outcomes in a randomized, placebo controlled study of COPD patients treated with anti-ST2.

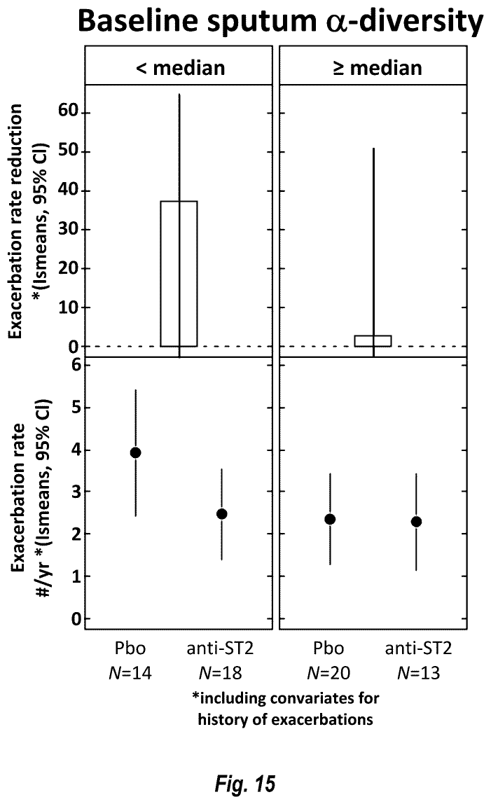

Baseline sputum 16s rRNA sequencing data was obtained for 65 of 81 participants of the ST2OP study. Annualized exacerbation rate for placebo treated subjects with baseline α-diversity below the median was higher than placebo subjects with baseline α-diversity greater than or equal to the median (3.9 [2.4, 5.4] versus 2.3 [1.3, 3.4] exacerbations per year) (FIG. 15). Furthermore, subjects with baseline α-diversity below the median derived greater placebo adjusted treatment benefit (37.1 [−12.1, 64.7] versus 2.5 [−93.7, 50.9] percent relative reduction) (FIG. 15). The median baseline α-diversity index in the study was 3.42.

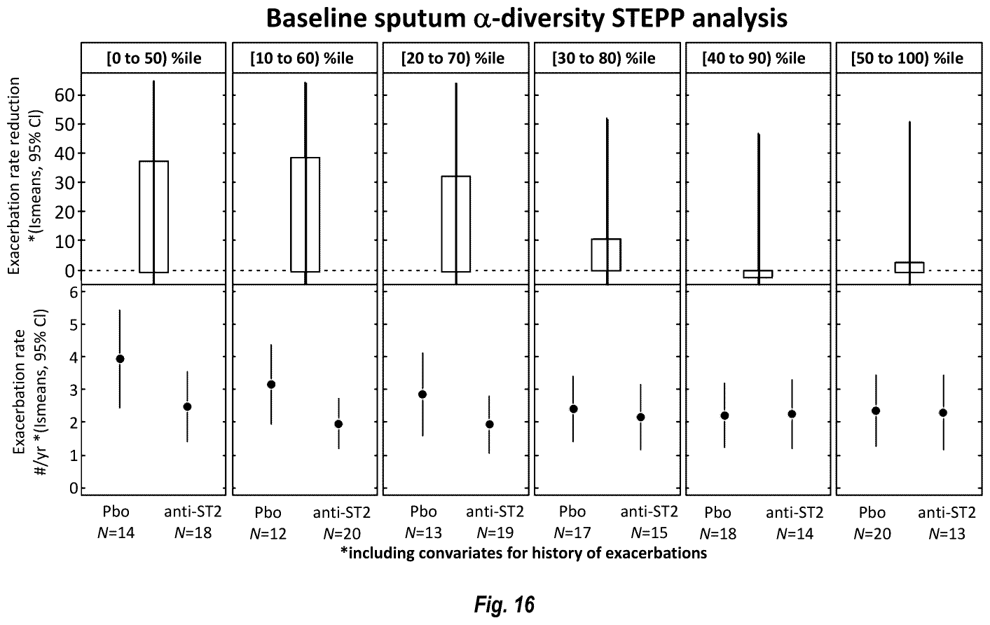

As baseline α-diversity is a continuous as opposed to a categorical biomarker without any established reference ranges, STEPP analysis (Lazar A A, J. Clin. Oncol. 2010 Oct. 10; 28(29):4539-44) was performed to better understand the relationship between pre-treatment α-diversity levels and treatment effect (FIG. 16). STEPP analysis supports that increasing range of α-diversity levels is associated with greater treatment effect and prognostic for exacerbations for placebo treated subjects.

Thus, the pretreatment lung microbial α-diversity is prognostic for increased COPD exacerbations and treatment benefit from anti-ST2 therapy. These data highlight that molecular pathways and factors underlying COPD exacerbations are heterogeneous and therapeutic strategies targeting IL-33 biology alone may have the greatest efficacy subjects with lung dysbiosis.

TABLE of
Certain Sequences

| SEQ | | |
|---|---|---|
| ID | | |
| NO | Name | Sequence |
| 1 | Ab2 heavy chain | NYWIG |
| | complementarity | |
| | determining region 1 | |
| | (H-CDR1); Kabat | |
| 2 | Ab2 H-CDR2; Kabat | IIYPGNSDTRFSPSFQ |
| 31 | Ab2 H-CDR2 | IIYPGNSDTRESPSFQG |
| | alternative; Kabat | |
| 3 | Ab2 H-CDR3; Kabat | HGTSSDYYGLDV |
| 4 | Ab2 light chain CDR1 | QASQDISNYLN |
| | (L-CDR1); Kabat | |
| 5 | Ab2 L-CDR2; Kabat | DASNLET |
| 6 | Ab2 L-CDR3; Kabat | QQDDNFPLT |
| 7 | Ab2 heavy chain | EVOLVQSGAEVKKPGESLKISCKGSGYSFTNYWIGWVRQMPGKGLEWMGI |
| | variable domain | IYPGNSDTRFSPSFQGQVTISADKSITTAYLQWSSLKASDTAMYYCARHG |
| | | TSSDYYGLDVWGQGTTVTVSS |
| 8 | Ab2 light chain | DIQMTQSPSSLSASVGDRVTITCQASQDISNYLNWYQQKPGKAPKLLIYD |
| | variable domain | ASNLETGVPSRFSGSGSGTDFTFTISSLQPEDIATYYCQQDDNFPLTFGG |
| | | GTKVEIKR |
| 9 | Ab2 heavy chain | EVOLVQSGAE VKKPGESLKI SCKGSGYSFT NYWIGWVRQM |
| | | PGKGLEWMGI IYPGNSDTRF SPSFQGQVTI SADKSITTAY |
| | | LOWSSLKASD TAMYYCARHG TSSDYYGLDV WGQGTTVTVS |
| | | SASTKGPSVF PLAPCSRSTS ESTAALGCLV KDYFPEPVTV |
| | | SWNSGALTSG VHTFPAVLOS SGLYSLSSVV TVPSSNFGTQ |
| | | TYTCNVDHKP SNTKVDKTVE RKCCVECPPC PAPPVAGPSV |
| | | FLFPPKPKDT LMISRTPEVT CVVVDVSHED PEVQFNWYVD |
| | | GVEVHNAKTK PREEQFNSTF RVVSVLTVVH QDWLNGKEYK |
| | | CKVSNKGLPA PIEKTISKTK GOPREPQVYT LPPSREEMTK |
| | | NQVSLTCLVK GFYPSDIAVE WESNGOPENN YKTTPPMLDS |
| | | DGSFFLYSKL TVDKSRWQQG NVFSCSVMHE ALHNHYTOKS |
| | | LSLSPGK |
| 32 | Ab2 heavy chain | EVQLVQSGAE VKKPGESLKI SCKGSGYSFT NYWIGWVROM |
| | lacking C-terminal | PGKGLEWMGI IYPGNSDTRF SPSFQGQVTI SADKSITTAY |
| | lysine | LQWSSLKASD TAMYYCARHG TSSDYYGLDV WGQGTTVTVS |
| | | SASTKGPSVF PLAPCSRSTS ESTAALGCLV KDYFPEPVTV |
| | | SWNSGALTSG VHTFPAVLQS SGLYSLSSVV TVPSSNFGTQ |
| | | TYTCNVDHKP SNTKVDKTVE RKCCVECPPC PAPPVAGPSV |
| | | FLFPPKPKDT LMISRTPEVT CVVVDVSHED PEVQFNWYVD |
| | | GVEVHNAKTK PREEQFNSTF RVVSVLTVVH QDWLNGKEYK |
| | | CKVSNKGLPA PIEKTISKTK GQPREPQVYT LPPSREEMTK |
| | | NQVSLTCLVK GFYPSDIAVE WESNGQPENN YKTTPPMLDS |
| | | DGSFFLYSKL TVDKSRWQQG NVFSCSVMHE ALHNHYTQKS |
| | | LSLSPG |
| 10 | Ab2 light chain | DIQMTQSPSS LSASVGDRVT ITCQASQDIS NYLNWYQQKP |
| | | GKAPKLLIYD ASNLETGVPS RFSGSGSGTD FTFTISSLOP |
| | | EDIATYYCQQ DDNFPLTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSEN RGEC |
| 35 | Ab2 H-CDR1; IMGT | GYSFTNYW |
| 36 | Ab2 H-CDR2; IMGT | IYPGNS |
| 37 | Ab2 H-CDR3; IMGT | YCARHGTSSDYYGLDV |
| 38 | Ab2 L-CDR1; IMGT | QDISNY |
| 39 | Ab2 L-CDR2; IMGT | DAS |
| 40 | Ab2 L-CDR3; IMGT | YCQQDDNFPLT |
| 11 | Ab5 H-CDR1; Kabat | SYDMY |
| 12 | Ab5 H-CDR2; Kabat | GIDTVGDTYYPDSVKG |
| 13 | Ab5 H-CDR3; Kabat | GIYGDFYYYGLDV |
| 14 | Ab5 L-CDR1; Kabat | RSSQSLLYSDGNNYLD |
| 15 | Ab5 L-CDR2; Kabat | LGSNRAS |
| 16 | Ab5 L-CDR3; Kabat | MQALQTLT |
| 17 | Ab5 heavy chain | EVQLVESGGGWVQPGGSLRLSCAASGFTFSSYDMYWVRQATGKGLEWVSG |
| | variable domain | IDTVGDTYYPDSVKGRFTISRENAKNSVYLQMNTLRAGDTAVYYCVRGIY |
| | | GDFYYYGLDVWGHGTTVTVS |
| 18 | Ab5 light chain | DIVMTQSPLSLPVTPGEPASISCRSSQSLLYSDGNNYLDWYLQKPGQSPH |
| | variable domain | LLIYLGSNRASGVPDRFSGSGSGTDFTLKISRVEADDVGVYYCMQALQTL |
| | | TFGGGTKVEIKR |
| 19 | Ab5 heavy chain | EVQLVESGGGWVQPGGSLRLSCAASGFTFSSYDMYWVRQATGKGLEWVSG |
| | | IDTVGDTYYPDSVKGRFTISRENAKNSVYLOMNTLRAGDTAVYYCVRGIY |
| | | GDFYYYGLDVWGHGTTVTVSSASTKGPSVFPLAPCSRSTSESTAALGCLV |
| | | KDYFPEPVTVSWNSGALTSGVHTFPAVLOSSGLYSLSSVVTVPSSNFGTQ |
| | | TYTCNVDHKPSNTKVDKTVERKCCVECPPCPAPPVAGPSVFLFPPKPKDT |
| | | LMISRTPEVTCVVVDVSHEDPEVQFNWYVDGVEVHNAKTKPREEQFNSTF |
| | | RVVSVLTVVHQDWLNGKEYKCKVSNKGLPAPIEKTISKTKGQPREPQVYT |
| | | LPPSREEMTKNQVSLTCLVKGFYPSDIAVEWESNGQPENNYKTTPPMLDS |
| | | DGSFFLYSKLTVDKSRWQQGNVFSCSVMHEALHNHYTQKSLSLSPGK |
| 33 | Ab5 heavy chain | EVOLVESGGGWVQPGGSLRLSCAASGFTFSSYDMYWVRQATGKGLEWVSG |
| | lacking C-terminal | IDTVGDTYYPDSVKGRFTISRENAKNSVYLOMNTLRAGDTAVYYCVRGIY |
| | lysine | GDFYYYGLDVWGHGTTVTVSSASTKGPSVFPLAPCSRSTSESTAALGCLV |
| | | KDYFPEPVTVSWNSGALTSGVHTFPAVLOSSGLYSLSSVVTVPSSNFGTQ |
| | | TYTCNVDHKPSNTKVDKTVERKCCVECPPCPAPPVAGPSVFLFPPKPKDT |
| | | LMISRTPEVTCVVVDVSHEDPEVQFNWYVDGVEVHNAKTKPREEQFNSTF |
| | | RVVSVLTVVHQDWLNGKEYKCKVSNKGLPAPIEKTISKTKGQPREPQVYT |
| | | LPPSREEMTKNQVSLTCLVKGFYPSDIAVEWESNGQPENNYKTTPPMLDS |
| | | DGSFFLYSKLTVDKSRWQQGNVFSCSVMHEALHNHYTQKSLSLSPG |
| 20 | Ab5 light chain | DIVMTQSPLSLPVTPGEPASISCRSSQSLLYSDGNNYLDWYLOKPGQSPH |
| | | LLIYLGSNRASGVPDRFSGSGSGTDFTLKISRVEADDVGVYYCMQALQTL |
| | | TFGGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACEV |
| | | THQGLSSPVTKSENRGEC |
| 21 | Ab7 H-CDR1; Kabat | TYDMH |
| 22 | Ab7 H-CDR2; Kabat | AIDLAGDTYYPGSVKG |
| 23 | Ab7 H-CDR3; Kabat | GGDGYNYDYYGIDV |
| 24 | Ab7 L-CDR1; Kabat | RSSQSLLHSDGYHYLD |
| 25 | Ab7 L-CDR2; Kabat | LGSNRAS |
| 26 | Ab7 L-CDR3; Kabat | MQALQTLT |
| 27 | Ab7 heavy chain | EVOLVESGGGLVQPGGSLRLSCAASGFTFSTYDMHWVRQTTGKGLEWVSA |
| | variable domain | IDLAGDTYYPGSVKGRFTISREDAKNSLYLQMNSLRAGDTAVYYCARGGD |
| | | GYNYDYYGIDVWGQGTTVTVSS |
| 28 | Ab7 light chain | DIVMTQSPLSLPVTPGEPASISCRSSQSLLHSDGYHYLDWYLQKPGQSPQ |
| | variable domain | LLIYLGSNRASGVPDRFSGSGSGTDFTLNISRVEAEDVGVYYCMQALQTL |
| | | TFGGGTKVEIKR |
| 29 | Ab7 heavy chain | EVOLVESGGGLVQPGGSLRLSCAASGFTFSTYDMHWVRQTTGKGLEWVSA |
| | | IDLAGDTYYPGSVKGRFTISREDAKNSLYLQMNSLRAGDTAVYYCARGGD |
| | | GYNYDYYGIDVWGQGTTVTVSSASTKGPSVFPLAPCSRSTSESTAALGCL |
| | | VKDYFPEPVTVSWNSGALTSGVHTFPAVLQSSGLYSLSSVVTVPSSNFGT |
| | | QTYTCNVDHKPSNTKVDKTVERKCCVECPPCPAPPVAGPSVFLFPPKPKD |
| | | TLMISRTPEVTCVVVDVSHEDPEVQFNWYVDGVEVHNAKTKPREEQFNST |
| | | FRVVSVLTVVHQDWLNGKEYKCKVSNKGLPAPIEKTISKTKGQPREPQVY |
| | | TLPPSREEMTKNQVSLTCLVKGFYPSDIAVEWESNGQPENNYKTTPPMLD |
| | | SDGSFFLYSKLTVDKSRWQQGNVFSCSVMHEALHNHYTQKSLSLSPGK |
| 34 | Ab7 heavy chain | EVQLVESGGGLVQPGGSLRLSCAASGFTFSTYDMHWVRQTTGKGLEWVSA |
| | lacking C-terminal | IDLAGDTYYPGSVKGRFTISREDAKNSLYLQMNSLRAGDTAVYYCARGGD |
| | lysine | GYNYDYYGIDVWGQGTTVTVSSASTKGPSVFPLAPCSRSTSESTAALGCL |
| | | VKDYFPEPVTVSWNSGALTSGVHTFPAVLQSSGLYSLSSVVTVPSSNFGT |
| | | QTYTCNVDHKPSNTKVDKTVERKCCVECPPCPAPPVAGPSVFLFPPKPKD |
| | | TLMISRTPEVTCVVVDVSHEDPEVQFNWYVDGVEVHNAKTKPREEQFNST |
| | | FRVVSVLTVVHQDWLNGKEYKCKVSNKGLPAPIEKTISKTKGQPREPQVY |
| | | TLPPSREEMTKNQVSLTCLVKGFYPSDIAVEWESNGQPENNYKTTPPMLD |
| | | SDGSFFLYSKLTVDKSRWQQGNVFSCSVMHEALHNHYTQKSLSLSPG |
| 30 | Ab7 light chain | DIVMTQSPLSLPVTPGEPASISCRSSOSLLHSDGYHYLDWYLQKPGOSPQ |
| | | LLIYLGSNRASGVPDRFSGSGSGTDFTLNISRVEAEDVGVYYCMQALQTL |
| | | TFGGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACEV |
| | | THQGLSSPVTKSENRGEC |
| 41 | rs10206753 | AGAAAGGCCTCTAGTTTGACTCCCT[C/T]GGCTGCCCAGAAGCAATAGT |
| | | GCCTG |

### REFERENCES

Abston E D, Baron J G, Cihakova D, et al. IL-33 independently induces eosinophilic pericarditis and cardiac dilation: ST2 improves cardiac function. Circ Heart Fail 2012; 5:366-75.
AbuDagga A, Sun S X, Tan H, et al. Healthcare utilization and costs among chronic bronchitis patients treated with maintenance medications from a US managed care population. J Med Econ 2013; 16:421-9.
Anzueto A. Impact of exacerbations on COPD. Eur Respri Rev 2010; 19:113-18.
Byers D E, Alexander-Brett J, Patel A C, et al. Long-term IL-33-producing epithelial progenitor cells in chronic obstructive lung disease. J Clin Invest 2013; 123:3967-82.
Campbell R L, Li J T C, Nicklas R A, et al. Emergency department diagnosis and treatment of anaphylaxis: a practice parameter. Ann Allergy Asthma Immunol 2014; 113:599-608.
Cayrol, C. and J. P. Girard, Interleukin-33 (IL-33): A nuclear cytokine from the IL-1 family. Immunol Rev, 2018. 281(1): p. 154-168.
Cayrol, C. and J. P. Girard, IL-33: an alarmin cytokine with crucial roles in innate immunity, inflammation and allergy. Curr Opin Immunol, 2014. 31: p. 31-7.
Cazzola M, Ora J, Puxeddu E. Dual bronchodilation and exacerbations of COPD. J Thorac Dis 2016; 8:2383-6.
Celli B R, Thomas N E, Anderson J A, et al. Effect of pharmacotherapy on rate of decline of lung function in chronic obstructive pulmonary disease: results from TORCH study. Am J Respir Crit Care Med 2008; 178:332-8.
Corominas M, Gastaminza G, Lobera T. Hypersensitivity reactions to biological drugs. J Investig Allergol Clin Immunol 2014; 24:212.
Decramer M, Celli B, Kesten S, et al. Effect of tiotropium on outcomes in patients with moderate chronic obstructive pulmonary disease (UPLIFT): a prespecified subgroup analysis of a randomised controlled trial. Lancet 2009; 374:1171-8.
Demyanets S, Konya V, Kastl S P, et al. Interleukin-33 induces expression of adhesion molecules and inflammatory activation in human endothelial cells and in human atherosclerotic plaques. Arterioscler Thromb Vase Biol 2011; 31:2080-89.
Diette G B, Dalal A A, D'Souza A O, et al. Treatment patterns of chronic obstructive pulmonary disease in employed adults in the United States. Int J Chron Obstruct Pulmon Dis 2015; 415-22.
Ford, E. S., et al., COPD surveillance—United States, 1999-2011. Chest, 2013. 144(1): p. 284-305.
The global burden of disease. [Report] [cited 2017 Dec. 12, 2017]; Available on the Internet at www.who.int/respiratory/copd/en/.
[GOLD 2017] Global Strategy for the Diagnosis, Management and Prevention of COPD, Global Initiative for Chronic Obstructive Lung Disease (GOLD). 2017 [cited 2017 Feb. 10, 2017]; Available from: http://goldcopd.org.
[GOLD 2021] Global Initiative for Chronic Obstructive Lung Disease. Global strategy for the diagnosis, management, and prevention of chronic obstructive pulmonary disease, 2021 report. 2021 [cited 11 Feb. 2021]. Available from on the Internet at goldcopd.org/wp-content/uploads/2020/11/GOLD-REPORT-2021-v1.1-25Nov20 WMV.pdf.
Graham B L, Steenbruggen I, Miller M R, et al. Standardization of Spirometry 2019 Update. An Official American Thoracic Society and European Respiratory Society Technical Statement. Am J Respir Crit Care Med 2019; 200:e70-e88.
Guralnik J M, Simonsick E M, Ferrucci L. A short physical performance battery assessing lower extremity function: association with self-reported disability and prediction of mortality and nursing home admission. J Gerontol 1994; 49:M85-94.
Halpin D M G, Decramer M, Celli B, et al. Exacerbation frequency and course of COPD. Int J Chron Obstruct Pulmon Dis 2012; 7:653-61.
Han M K, Quibrera P M, Carretta E E, et al. Frequency of exacerbations in patients with chronic obstructive pulmonary disease: an analysis of the SPIROMICS cohort. Lancet Respir Med 2017; 5:619-26.
Hayakawa H, Hayakawa M, Kume A, et al. Soluble ST2 blocks interleukin-33 signaling in allergic airway inflammation. J Biol Chem 2007; 282:26369-380.
Hogg J C, Chu F, Utokaparch S, et al. The nature of small-airway obstruction in chronic obstructive pulmonary disease. N Engl J Med 2004; 350:2645-53.
Hurlbert, S. H. (1971). The nonconcept of species diversity: a critique and alternative parameters. Ecology 52, 577-586.
Hurst J R, Vestbo J, Anzueto A, et al. Susceptibility to exacerbation in chronic obstructive pulmonary disease. N Engl J Med 2010; 363:1128-38.
Jackson, D. J., et al., IL-33-dependent type 2 inflammation during rhinovirus-induced asthma exacerbations in vivo. Am J Respir Crit Care Med, 2014. 190(12): p. 1373-82.
Jones P W, Harding G, Berry P, et al. Development and first validation of the COPD Assessment Test. Eur Respir J 2009; 34:648-54.
Jones S E, Kon S S C, Canavan J L, et al. The five-repetition sit-to-stand test as a functional outcome measure in COPD. Thorax 2013; 68:1015-20.
Kaur, D., et al., IL-33 drives airway hyper-responsiveness through IL-13-mediated mast cell: airway smooth muscle crosstalk. Allergy, 2015. 70(5): p. 556-67.
Kearley J, Silver J S, Sanden C, et al. Cigarette smoke silences innate lymphoid cell function and facilitates an exacerbated type I interleukin-33-dependent response to infection. Immunity 2015; 42:566-79.
Landis S H, Muellerova H, Mannino D M, et al. Continuing to Confront COPD International Patient Survey: methods, COPD prevalence, and disease burden in 2012-2013. Int J Chron Obstruct Pulmon Dis 2014; 9:597-611.
Leidy N K, Wilcox T K, Jones P W, et al., EXACT-PRO Study Group. Development of the EXAcerbations of Chronic Obstructive Pulmonary Disease Tool (EXACT): a patient-reported outcome (PRO) measure. Value Health 2010; 13:965-75.
Leidy N K, Murray L T, Monz B U, et al. Measuring respiratory symptoms of COPD: performance of the EXACT-Respiratory Symptoms Tool (E-RS) in three clinical trials. Respir Res 2014; 15:124.
Liew F Y, Girard J-P, Turnquist H R. Interleukin-33 in health and disease. Nat Rev Immunol 2016; 16:676-89.
Louten J, Rankin A L, Li Y, et al. Endogenous IL-33 enhances Th2 cytokine production and T-cell responses during allergic airway inflammation. Int Immunol 2011; 23:307-15.
Martin P, Palmer G, Rodriquez E, et al. Atherosclerosis severity is not affected by a deficiency in IL-33/ST2 signaling. Immun Inflamm Dis 2015; 3:239-46.
McLaren J E, Michael D R, Salter R C, et al. IL-33 reduces macrophage foam cell formation. J Immunol 2010; 185:1222-29.
Meguro M, Barley E A, Spencer S, et al. Development and validation of an improved, COPD-specific version of the St. George Respiratory Questionnaire. Chest 2007; 132:456-63.
Miller A M, Xu D, Asquith D L, et al. IL-33 reduces the development of atherosclerosis. J Exp Med 2008; 205:339-46.
Miravitlles M, Ferrer M, Pont A, et al. Effect of exacerbations on quality of life in patients with chronic obstructive pulmonary disease: a 2 year follow up study. Thorax 2004; 59:387-95.
Molofsky A B, Savage A K, Locksley R M. Interleukin-33 in tissue homeostasis, injury, and inflammation. Immunity 2015; 42:1005-19.
Nabe T. Interleukin (IL)-33: new therapeutic target for atopic diseases. J Pharmacol Sci 2014; 126:85-91.
Patalano F, Banerji D, D'Andrea P D, et al. Addressing unmet needs in the treatment of COPD. Eur Respir Rev 2014; 23:333-44.
Ramirez-Carrozzi V, Dressen A, Lupardus P, Yaspan B, Pappu R. Functional analysis of protective IL1RL1 variants associated with asthma risk. J Allergy Clin Immunol. 2014; 135(4):1080-1083.e3.
Ravanetti L, Dijkhuis A, Dekker T. IL-33 drives influenza-induced asthma exacerbations by halting innate and adaptive antiviral immunity; J Allergy Clin Immunol 2019; 143:1355-70.
Sabatine M S, Morrow, DA, Higgins L J, et al. Complementary roles for biomarkers of biomechanical strain ST2 and N-terminal prohormone B-type natriuretic peptide in patients with ST-elevation myocardial infarction. Circulation 2008; 117:1936-44.
Sampson H A, Munoz-Furlong A, Campbell R L, et al. Second symposium on the definition and management of anaphylaxis: summary report. Second National Institute of Allergy and Infectious Disease/Food Allergy and Anaphylaxis Network symposium. J Allergy Clin Immunol. 2006; 117:391-7.
Sanada S, Hakuno D, Higgins L J, et al. IL-33 and ST2 comprise a critical biomechanically induced and cardioprotective signaling system. J Clin Invest 2007; 117:1538-49.
Scanlon S T, McKenzie A N. Type 2 innate lymphoid cells: new players in asthma and allergy. Curr Opin Immunol 2012; 24:707-12.
Seki K, Sanada S, Kudinova A Y, et al. Interleukin-33 prevents apoptosis and improves survival after experimental myocardial infarction through ST2 signaling. Circ Heart Fail 2009; 2:684-91.
Shah R V, Chen-Toumoux A A, Picard M H, et al. Serum levels of the interleukin-1 receptor family member ST2, cardiac structure and function, and long-term mortality in patients with acute dyspnea. Circ Heart Fail 2009; 2:311-19.
Sims J E, Smith D E. The IL-1 family: regulators of immunity. Nat Rev Immunol 2010; 10:89-102.
Solem C T, Sun S X, Sudharshan L, et al. Exacerbation-related impairment of quality of life and work productivity in severe and very severe chronic obstructive pulmonary disease. Int J Chron Obstruct Pulmon Dis 2013; 8:641-52.
Wasserman A, Ben-Shoshan J, Entin-Meer M, et al. Interleukin-33 augments Treg cell levels: a flaw mechanism in atherosclerosis. Isr Med Assoc J 2012; 14:620-23.
Weir R A, Miller A M, Murphy G E J, et al. Serum soluble ST2: a potential novel mediator in left ventricular and infarct remodeling after acute myocardial infarction. J Am Coll Cardiol 2010; 55:243-50.
Werder R B, Zhang V, Lynch J P. Chronic IL-33 expression predisposes to virus-induced asthma exacerbations by increasing type 2 inflammation and dampening antiviral immunity. J Allergy Clin Immunol 2018; 141:1607-19.
[WHO] World Health Organization. The top 10 causes of death the top ten causes of death [resource on the Internet]. 2020 [cited 30 Mar. 2021]. Available on the Internet at www.who.int/news-room/fact-sheets/detail/the-top-10-causes-of-death.
[WHO] World Health Organization. Burden of COPD [resource on the Internet]. 2021 [cited 30 Mar. 2021]. Available on the Internet at: www.who.int/respiratory/copd/burden/en.

1. A method of treating chronic obstructive pulmonary disease (COPD) in a patient comprising administering:
a) 476 mg of an ST2 antagonist to the patient on Day 1 of a treatment period,
b) an effective amount of an ST2 antagonist to the patient, wherein the patient is selected for treatment on the basis of the genotype of the patient determined to comprise a TT allele or CT allele at polymorphism rs10206753;
c) an effective amount of an ST2 antagonist to the patient, wherein the patient is selected for treatment on the basis of the level of sST2 in a sample derived from the patient is determined to be at or above a reference level of sST2;
d) an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient; or
e) an effective amount of an ST2 antagonist to the patient, wherein the patient is selected for treatment on the basis of the level of baseline α-diversity in a sample derived from the patient is determined to be below a reference level of alpha-diversity index.

2. A method of reducing the frequency of moderate to severe exacerbations in a patient having COPD comprising administering:
a) 476 mg of an ST2 antagonist to the patient on Day 1 of a treatment period;
b) an effective amount of an ST2 antagonist to achieve a clinical improvement of at least 10%, at least 20%, at least 21%, at least 22%, at least 25%, at least 30%, at least 35%, at least 40% or at least 45% annualized exacerbation rate reduction than standard of care (SOC);
c) an ST2 antagonist to the patient in an amount effective to achieve a greater clinical improvement in the number of exacerbations than standard of care (SOC), said patient having a baseline blood eosinophil count <300 eosinophils/μL;
d) an ST2 antagonist to the patient in an amount effective to achieve a greater clinical improvement in the number of exacerbations than SOC, said patient having a baseline blood eosinophil count ≤170 eosinophils/μL;
e) an ST2 antagonist to the patient in an amount effective to achieve a greater clinical improvement in the number of exacerbations than SOC, said patient having a post-bronchodilator (post-BD) spirometry measurement of <0.7 as measured by forced expiratory volume in one second (FEV1) and/or forced vital capacity (FVC);
f) an ST2 antagonist to the patient in an amount effective to achieve a greater clinical improvement in the number of exacerbations than SOC, said patient having a modified Medical Research Council (mMRC) dyspnea scale score ≥2 and a COPD assessment test score (CAT) of ≥10;
g) an ST2 antagonist to the patient in an amount effective to achieve a reduction of at least about 25%, e.g., at least about 30%, at least about 35%, at least about 40%, or at least about 45% in the number of moderate to severe exacerbations at 50 weeks and/or 52 weeks from the start of treatment, as measured by annualized exacerbation rate as compared to SOC;
h) an effective amount of an ST2 antagonist to the patient, wherein the patient is selected for treatment on the basis of the genotype of the patient determined to comprise a TT allele or CT allele at polymorphism rs10206753;
i) an effective amount of an ST2 antagonist to the patient, wherein the patient is selected for treatment on the basis of the level of sST2 in a sample derived from the patient is determined to be at or above a reference level of sST2;
j) an effective amount of an ST2 antagonist to the patient, wherein the patient is selected for treatment on the basis of the level of one or more biomarkers selected from eosinophils, IL-33 pathway markers, inflammatory proteins (e.g., fibrinogen, C-reactive protein) and single nucleotide polymorphisms (SNPs) of genes related to COPD (e.g., IL1RL1, IL33) in a sample derived from the patient; or
k) an effective amount of an ST2 antagonist to the patient, wherein the patient is selected for treatment on the basis of the level of baseline α-diversity in a sample derived from the patient is determined to be below a reference level of alpha-diversity.

3.-7. (canceled)

8. A method of treating or preventing COPD comprising administering an ST2 antagonist to a patient in an amount effective to achieve a greater clinical improvement than SOC as measured by patient reported outcome (PRO), wherein the PRO is an improvement of at least about 1, at least about 2, at least about 3, or at least about 4 points from baseline in a St. George's Respiratory Questionnaire for COPD patients (SGRQ-C) at 4 weeks, 12 weeks, 24 weeks, 36 weeks, or 48 weeks from the start of treatment.

9. A method for maintaining and/or improving lung function in a patient having COPD comprising administering an ST2 antagonist to the patient in an amount effective to achieve a greater clinical improvement in lung function than SOC, wherein clinical improvement is demonstrated by a mean difference compared to baseline of at least 0.04 L, 0.05 L, 0.06 L, 0.07 L, 0.08 L, or 0.09 L as measured by post-BD FEV1 at 4 weeks, 12 weeks, 24 weeks, 36 weeks, or 48 weeks from the start of treatment or wherein clinical improvement is demonstrated by a mean difference compared to baseline of at least about 5% as measured by post-BD FEV1 at 4 weeks, 12 weeks, 24 weeks, 36 weeks, or 48 weeks from the start of treatment.

10. A method of improving baseline blood eosinophil count in a patient having COPD comprising administering an ST2 antagonist to the patient in an amount effective to reduce mean blood eosinophil count by at least about 25%, e.g., at least about 30%, at least about 35%, at least about 40%, at least about 45% compared to baseline, after about 4 weeks, 12 weeks, 24 weeks, 36 weeks, or 48 weeks following administration of a first dose of an ST2 antagonist.

11.-22. (canceled)

23. The method of claim 1, wherein the reference level of baseline α-diversity is an α-diversity index of about 3.4, as calculated by Shannon-Weaver method; or wherein the reference level of baseline α-diversity is an α-diversity index is in the range of about 0 to 5 as calculated by Shannon-Weaver method.

24. (canceled)

25. The method ofany one of claim 1, wherein the sample is a blood, serum, plasma, or urine sample.

26. (canceled)

27. The method of claim 1, comprising administering 476 mg of the ST2 antagonist to the patient on Day 1 of a treatment period.

28. The method of claim 1, comprising administering the ST2 antagonist every 2 weeks, or every 4 weeks.

29. (canceled)

30. The method of claim 1, comprising administering 476 mg of the ST2 antagonist every 2 weeks, or every 4 weeks.

31. (canceled)

32. (canceled)

33. The method of claim 1, comprising administering 490 mg of the ST2 antagonist every 2 weeks, or every 4 weeks.

34. (canceled)

35. (canceled)

36. The method of claim 1, wherein the patient:
a) has had two or more moderate-to-severe exacerbations within a 12-month period prior to treatment;
b) has a mMRC dyspnea score ≥2;
c) has post-bronchodilator FEV1≥20 and <80% of predicted normal value; or
d) has post-bronchodilator FEV1/FVC<0.7.

37. (canceled)

38. (canceled)

39. (canceled)

40. The method of claim 1, which achieves a greater improvement in clinical outcome compared to standard of care (SOC).

41. The method of claim 1, which reduces the number of moderate to severe exacerbations:
a) as measured by annualized exacerbation rate reduction (AERR) as compared to SOC at 4 weeks, 12 weeks, 24 weeks, 36 weeks, 48 weeks, 50 weeks, or 52 weeks from the start of treatment; or
as measured by AERR by at least about 25%, at least about 30%, at least about 35%, at least about 40%, or at least about 45% as compared to SOC.

42. (canceled)

43. The method of claim 1, which increases the time to first moderate or severe COPD exacerbation as compared to SOC.

44. The method of claim 1, which improves absolute change from baseline in health-related quality of life (HRQoL):
a) as assessed through a St. George's Respiratory Questionnaire for COPD patients (SGRQ-C) total score as compared to SOC at 4 weeks, 12 weeks, 24 weeks, 36 weeks, 48 weeks, 50 weeks, or 52 weeks from the start of treatment; or
b) as defined as a decrease from baseline of ≥4 points in SGRQ-C total score at 4 weeks, 12 weeks, 24 weeks, 36 weeks, 48 weeks, 50 weeks, or 52 weeks from the start of treatment.

45. (canceled)

46. The method of claim 1, which improves:
a) a absolute change from baseline post-bronchodilator in forced expiratory volume in one second (FEV1) (liters) at 4 weeks, 12 weeks, 24 weeks, 36 weeks, 48 weeks, 50 weeks, or 52 weeks from the start of treatment,
b) absolute change from baseline in Evaluating Respiratory Symptoms in COPD (ERS:COPD) total score from baseline at 4 weeks, 12 weeks, 24 weeks, 36 weeks, 48 weeks, 50 weeks, or 52 weeks from the start of treatment;
c) the annualized rate of severe COPD exacerbations at 4 weeks, 12 weeks, 24 weeks, 36 weeks, 48 weeks, 50 weeks, or 52 weeks from the start of treatment;
d) absolute change from baseline in five-repetition sit-to-stand test (5STS) time (seconds) at 4 weeks, 12 weeks, 24 weeks, 36 weeks, 48 weeks, 50 weeks, or 52 weeks from the start of treatment;
e) the annualized rate of EXAcerbations of Chronic Pulmonary Disease Tool and Evaluating Respiratory Symptoms in COPD (EXACT)-defined exacerbation events from baseline at 4 weeks, 12 weeks, 24 weeks, 36 weeks, 48 weeks, 50 weeks, or 52 weeks from the start of treatment;
f) an EXACT exacerbation event; or
g) at least one non-E-RS COPD domain from baseline at 4 weeks, 12 weeks, 24 weeks, 36 weeks, 48 weeks, 50 weeks, or 52 weeks from the start of treatment.

47.-52. (canceled)

53. The method of claim 46, wherein the non-E-RS COPD domain is tiredness/weakness, sleep disturbance, or fear/worry.

54. The method of claim 1, which improves the proportion of patients with HRQoL improvement, defined as a decrease from baseline of ≥4 points in SGRQ-C total score, at 4 weeks, 12 weeks, 24 weeks, 36 weeks, 48 weeks, 50 weeks, or 52 weeks from the start of treatment; or improves the proportion of patients with symptom improvement, defined as decrease from baseline of ≥2 points from baseline in E-RS:COPD total score, at 4 weeks, 12 weeks, 24 weeks, 36 weeks, 48 weeks, 50 weeks, or 52 weeks from the start of treatment.

55. (canceled)

56. The method of claim 1, which results in symptom improvement in the patient, defined as decrease from baseline of ≥2 points from baseline in E-RS:COPD total score, at 4 weeks, 12 weeks, 24 weeks, 36 weeks, 48 weeks, 50 weeks, or 52 weeks from the start of treatment.

57. The method of claim 1, which:
a) improves the E-RS:COPD cough and sputum domain from baseline at 4 weeks, 12 weeks, 24 weeks, 36 weeks, 48 weeks, 50 weeks, or 52 weeks from the start of treatment;
b) improves the E-RS:COPD breathlessness domain from baseline at 4 weeks, 12 weeks, 24 weeks, 36 weeks, 48 weeks, 50 weeks, or 52 weeks from the start of treatment;
c) improves the E-RS:COPD chest symptom domain from baseline at 4 weeks, 12 weeks, 24 weeks, 36 weeks, 48 weeks, 50 weeks, or 52 weeks from the start of treatment;
d) improves the absolute change from baseline in post-bronchodilator FEV1 (liters) at 4 weeks, 12 weeks, 24 weeks, 36 weeks, 48 weeks, 50 weeks, or 52 weeks from the start of treatment;
e) improves the annualized rate of moderate COPD exacerbations at 4 weeks, 12 weeks, 24 weeks, 36 weeks, 48 weeks, 50 weeks, or 52 weeks from the start of treatment;
f) improves the duration of hospital stay for severe COPD exacerbations;
g) reduces healthcare utilization for severe COPD exacerbations;
h) improves the proportion of severe COPD exacerbations requiring hospital readmission within 30 days;
i) improves the absolute change from baseline in residual volume/forced lung capacity ratio from baseline at 4 weeks, 12 weeks, 24 weeks, 36 weeks, 48 weeks, 50 weeks, or 52 weeks from the start of treatment;
j) improves the absolute change from baseline in daily step count at 4 weeks, 12 weeks, 24 weeks, 36 weeks, 48 weeks, 50 weeks, or 52 weeks from the start of treatment;
k) improves the absolute change from baseline in time in moderate and vigorous physical activity at 4 weeks, 12 weeks, 24 weeks, 36 weeks, 48 weeks, 50 weeks, or 52 weeks from the start of treatment;
l) improves the absolute change from baseline in COPD Assessment Test (CAT) score at 4 weeks, 12 weeks, 24 weeks, 36 weeks, 48 weeks, 50 weeks, or 52 weeks from the start of treatment;
m) improves the annualized rate of moderate and severe COPD exacerbations over a blinded treatment period;
n) improves health-related quality of life as measured by patient reported outcome (PRO) as compared to SOC;
o) improves PRO as assessed through SGRQ-C by at least about 1, at least about 2, at least about 3, or at least about 4 points from baseline at 4 weeks, 12 weeks, 24 weeks, 36 weeks, 48 weeks, 50 weeks, or 52 weeks from the start of treatment;
p) improves FEV1 by at least 5% from baseline at 4 weeks, 12 weeks, 24 weeks, 36 weeks, 48 weeks, 50 weeks, or 52 weeks from the start of treatment;
q) improves ERS:COPD total score from baseline by a decrease of at least about 2 points, at 4 weeks, 12 weeks, 24 weeks, 36 weeks, 48 weeks, 50 weeks, or 52 weeks from the start of treatment;
r improves the absolute change from baseline in rescue inhaler use at 4 weeks, 12 weeks, 24 weeks, 36 weeks, 48 weeks, 50 weeks, or 52 weeks from the start of treatment; or
s) improves the absolute change from baseline in nightly total sleep time at 4 weeks, 12 weeks, 24 weeks, 36 weeks, 48 weeks, 50 weeks, or 52 weeks from the start of treatment.

58.-75. (canceled)

76. The method of claim 1, wherein the ST2 antagonist is administered to the patient in combination with:
a) SOC;
b) inhaled corticosteroids (ICS);
c) ICS≥500 mcg/day fluticasone propionate dose-equivalent;
d) ICS plus long-acting betaagonist (LABA);
e) ICS≥500 mcg/day fluticasone propionate dose-equivalent plus LABA;
f) Long-acting muscarinic antagonist (LAMA) plus LABA;
g) ICS plus LAMA plus LABA; or
h) ICS≥500 mcg/day fluticasone propionate dose-equivalent plus LAMA plus LABA.

77. (canceled)

78. (canceled)

79. (canceled)

80. (canceled)

81. (canceled)

82. (canceled)

83. (canceled)

84. The method of claim 1, which is associated with acceptable safety outcome compared with standard of care.

85. The method of claim 84, wherein the safety outcome is selected from any one or more of: incidence and severity of adverse events, with severity determined according to the Division of AIDS Table for Grading the Severity of Adult and Pediatric Adverse Events, Version 2.1 (DAIDS Table v2.1) toxicity scale; change from baseline in targeted vital signs; and/or change from baseline in targeted clinical laboratory test results and ECGs.

86. The method of claim 1, wherein the patient:
a is a former smoker;
b) is a current smoker; or
c) has a baseline blood eosinophil count<300 eosinophils/μL.

87. (canceled)

88. (canceled)

89. The method of claim 1, wherein the ST2 antagonist:
a) is an inhibitor of ST2 biological activity;
b) binds to human ST2 or to human IL-33; or
c) is an anti-ST2 antibody.

90. (canceled)

91. (canceled)

92. The method of claim 89, wherein the ST2 antagonist is astegolimab.

93. The method of claim 89, wherein the anti-ST2 antibody is a human antibody.

94. The method of claim 93, wherein the anti-ST2 antibody comprises:
a) heavy chain complementarity determining region (H-CDR) 1 comprising an amino acid sequence that is at least 90% identical to the amino acid sequence of SEQ ID NO: 1, H-CDR2 comprising an amino acid sequence that is at least 90% identical to the amino acid sequence of SEQ ID NO: 2 or SEQ ID NO: 31, H-CDR3 comprising an amino acid sequence that is at least 90% identical to the amino acid sequence of SEQ ID NO: 3, light chain complementarity determining region (L-CDR) 1 comprising an amino acid sequence that is at least 90% identical to the amino acid sequence of SEQ ID NO: 4, L-CDR2 comprising an amino acid sequence that is at least 90% identical to the amino acid sequence of SEQ ID NO: 5, and L-CDR3 comprising an amino acid sequence that is at least 90% identical to the amino acid sequence of SEQ ID NO: 6;
b) heavy chain complementarity determining region (H-CDR) 1 comprising an amino acid sequence that is at least 90% identical to the amino acid sequence of SEQ ID NO: 35, H-CDR2 comprising an amino acid sequence that is at least 90% identical to the amino acid sequence of SEQ ID NO: 36, H-CDR3 comprising an amino acid sequence that is at least 90% identical to the amino acid sequence of SEQ ID NO: 37, light chain complementarity determining region (L-CDR) 1 comprising an amino acid sequence that is at least 90% identical to the amino acid sequence of SEQ ID NO: 38, L-CDR2 comprising an amino acid sequence that is at least 90% identical to the amino acid sequence of SEQ ID NO: 39, and L-CDR3 comprising an amino acid sequence that is at least 90% identical to the amino acid sequence of SEQ ID NO: 40;
c) heavy chain complementarity determining region (H-CDR) 1 comprising an amino acid sequence that is at least 90% identical to the amino acid sequence of SEQ ID NO: 11, H-CDR2 comprising an amino acid sequence that is at least 90% identical to the amino acid sequence of SEQ ID NO: 12, H-CDR3 comprising an amino acid sequence that is at least 90% identical to the amino acid sequence of SEQ ID NO: 13, light chain complementarity determining region (L-CDR) 1 comprising an amino acid sequence that is at least 90% identical to the amino acid sequence of SEQ ID NO: 14, L-CDR2 comprising an amino acid sequence that is at least 90% identical to the amino acid sequence of SEQ ID NO: 15, and L-CDR3 comprising an amino acid sequence that is at least 90% identical to the amino acid sequence of SEQ ID NO: 16; or
d) heavy chain complementarity determining region (H-CDR) 1 comprising an amino acid sequence that is at least 90% identical to the amino acid sequence of SEQ ID NO: 21, H-CDR2 comprising an amino acid sequence that is at least 90% identical to the amino acid sequence of SEQ ID NO: 22, H-CDR3 comprising an amino acid sequence that is at least 90% identical to the amino acid sequence of SEQ ID NO: 23, light chain complementarity determining region (L-CDR) 1 comprising an amino acid sequence that is at least 90% identical to the amino acid sequence of SEQ ID NO: 24, L-CDR2 comprising an amino acid sequence that is at least 90% identical to the amino acid sequence of SEQ ID NO: 25, and L-CDR3 comprising an amino acid sequence that is at least 90% identical to the amino acid sequence of SEQ ID NO: 26.

95. The method of claim 93, wherein the anti-ST2 antibody comprises:
a) heavy chain complementarity determining region (H-CDR) 1 comprising the amino acid sequence of SEQ ID NO: 1, H-CDR2 comprising the amino acid sequence of SEQ ID NO: 2 or SEQ ID NO: 31, H-CDR3 comprising the amino acid sequence of SEQ ID NO: 3, light chain complementarity determining region (L-CDR) 1 comprising the amino acid sequence of SEQ ID NO: 4, L-CDR2 comprising the amino acid sequence of SEQ ID NO: 5, and L-CDR3 comprising the amino acid sequence of SEQ ID NO: 6;
b) heavy chain complementarity determining region (H-CDR) 1 comprising the amino acid sequence of SEQ ID NO: 35, H-CDR2 comprising the amino acid sequence of SEQ ID NO: 36, H-CDR3 comprising the amino acid sequence of SEQ ID NO: 37, light chain complementarity determining region (L-CDR) 1 comprising the amino acid sequence of SEQ ID NO: 38, L-CDR2 comprising the amino acid sequence of SEQ ID NO: 39, and L-CDR3 comprising the amino acid sequence of SEQ ID NO: 40;
c) heavy chain complementarity determining region (H-CDR) 1 comprising the amino acid sequence of SEQ ID NO: 11, H-CDR2 comprising the amino acid sequence of SEQ ID NO: 12, H-CDR3 comprising the amino acid sequence of SEQ ID NO: 13, light chain complementarity determining region (L-CDR) 1 comprising the amino acid sequence of SEQ ID NO: 14, L-CDR2 comprising the amino acid sequence of SEQ ID NO: 15, and L-CDR3 comprising the amino acid sequence of SEQ ID NO: 16; or
d) heavy chain complementarity determining region (H-CDR) 1 comprising the amino acid sequence of SEQ ID NO: 21, H-CDR2 comprising the amino acid sequence of SEQ ID NO: 22, H-CDR3 comprising the amino acid sequence of SEQ ID NO: 23, light chain complementarity determining region (L-CDR) 1 comprising the amino acid sequence of SEQ ID NO: 24, L-CDR2 comprising the amino acid sequence of SEQ ID NO: 25, and L-CDR3 comprising the amino acid sequence of SEQ ID NO: 26.

96. The method of claim 93, wherein the anti-ST2 antibody comprises (a) heavy chain complementarity determining region (H-CDR) 1 comprising the amino acid sequence of SEQ ID NO: 1, H-CDR2 comprising the amino acid sequence of SEQ ID NO: 2 or SEQ ID NO: 31, H-CDR3 comprising the amino acid sequence of SEQ ID NO: 3, light chain complementarity determining region (L-CDR) 1 comprising the amino acid sequence of SEQ ID NO: 4, L-CDR2 comprising the amino acid sequence of SEQ ID NO: 5, and L-CDR3 comprising the amino acid sequence of SEQ ID NO: 6; or (b) heavy chain complementarity determining region (H-CDR) 1 comprising the amino acid sequence of SEQ ID NO: 35, H-CDR2 comprising the amino acid sequence of SEQ ID NO: 36, H-CDR3 comprising the amino acid sequence of SEQ ID NO: 37, light chain complementarity determining region (L-CDR) 1 comprising the amino acid sequence of SEQ ID NO: 38, L-CDR2 comprising the amino acid sequence of SEQ ID NO: 39, and L-CDR3 comprising the amino acid sequence of SEQ ID NO: 40.

97. The method of claim 93, wherein the anti-ST2 antibody comprises:
a) a heavy chain variable region comprising an amino acid sequence that is at least 90%, at least 95%, or at least 98% identical to the amino acid sequence of SEQ ID NO: 7 and a light chain variable region comprising an amino acid sequence that is at least 90%, at least 95%, or at least 98% identical to the amino acid sequence of SEQ ID NO: 8;
b) a heavy chain variable region comprising an amino acid sequence that is at least 90%, at least 95%, or at least 98% identical to the amino acid sequence of SEQ ID NO: 17 and a light chain variable region comprising an amino acid sequence that is at least 90%, at least 95%, or at least 98% identical to the amino acid sequence of SEQ ID NO: 18; or
c) a heavy chain variable region comprising an amino acid sequence that is at least 90%, at least 95%, or at least 98% identical to the amino acid sequence of SEQ ID NO: 27 and a light chain variable region comprising an amino acid sequence that is at least 90%, at least 95%, or at least 98% identical to the amino acid sequence of SEQ ID NO: 28.

98. The method of claim 93, wherein the anti-ST2 antibody comprises:
a) a heavy chain variable region comprising the amino acid sequence of SEQ ID NO: 7 and a light chain variable region comprising the amino acid sequence of SEQ ID NO: 8;
b) a heavy chain variable region comprising the amino acid sequence of SEQ ID NO: 17 and a light chain variable region comprising the amino acid sequence of SEQ ID NO: 18; or
c) a heavy chain variable region comprising the amino acid sequence of SEQ ID NO: 27 and a light chain variable region comprising the amino acid sequence of SEQ ID NO: 28.

99. The method of claim 93, wherein the anti-ST2 antibody comprises a heavy chain variable region comprising the amino acid sequence of SEQ ID NO: 7 and a light chain variable region comprising the amino acid sequence of SEQ ID NO: 8.

100. The method of claim 93, wherein the anti-ST2 antibody comprises:
a) a heavy chain comprising an amino acid sequence that is at least 90%, at least 95%, or at least 98% identical to the amino acid sequence of SEQ ID NO: 9 or SEQ ID NO: 32 and a light chain comprising an amino acid sequence that is at least 90%, at least 95%, or at least 98% identical to the amino acid sequence of SEQ ID NO: 10;
b) a heavy chain comprising an amino acid sequence that is at least 90%, at least 95%, or at least 98% identical to the amino acid sequence of SEQ ID NO: 19 and a light chain comprising an amino acid sequence that is at least 90%, at least 95%, or at least 98% identical to the amino acid sequence of SEQ ID NO: 20; or
c) a heavy chain comprising an amino acid sequence that is at least 90%, at least 95%, or at least 98% identical to the amino acid sequence of SEQ ID NO: 29 and a light chain comprising an amino acid sequence that is at least 90%, at least 95%, or at least 98% identical to the amino acid sequence of SEQ ID NO: 30.

101. The method of claim 93, wherein the anti-ST2 antibody comprises:
a) a heavy chain comprising the amino acid sequence of SEQ ID NO: 9 or SEQ ID NO: 32 and a light chain comprising the amino acid sequence of SEQ ID NO: 10;
b) a heavy chain comprising the amino acid sequence of SEQ ID NO: 19 and a light chain comprising the amino acid sequence of SEQ ID NO: 20; or
c) a heavy chain comprising the amino acid sequence of SEQ ID NO: 29 and a light chain comprising the amino acid sequence of SEQ ID NO: 30.

102. The method of claim 93, wherein the anti-ST2 antibody comprises a heavy chain comprising the amino acid sequence of SEQ ID NO: 9 or SEQ ID NO: 32 and a light chain comprising the amino acid sequence of SEQ ID NO: 10.

103. A kit comprising an ST2 antagonist and instructions to administer the ST2 antagonist to a patient in accordance with the method of claim 1.

104.-145. (canceled)


SEQUENCE LISTING (41 sequences)

SEQ ID NO: 1 - protein, 5 aa, synthetic construct
NYWIG

SEQ ID NO: 2 - protein, 16 aa, synthetic construct
IIYPGNSDTRFSPSFQ

SEQ ID NO: 3 - protein, 12 aa, synthetic construct
HGTSSDYYGLDV

SEQ ID NO: 4 - protein, 11 aa, synthetic construct
QASQDISNYLN

SEQ ID NO: 5 - protein, 7 aa, synthetic construct
DASNLET

SEQ ID NO: 6 - protein, 9 aa, synthetic construct
QQDDNFPLT

SEQ ID NO: 7 - protein, 121 aa, synthetic construct
EVQLVQSGAEVKKPGESLKISCKGSGYSFTNYWIGWVRQMPGKGLEWMGIIYPGNSDTRFSPSFQGQVTISADKSITTAYLQWSSLKASDTAMYYCARHGTSSDYYGLDVWGQGTTVTVSS

SEQ ID NO: 8 - protein, 108 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDDNFPLTFGGGTKVEIKR

SEQ ID NO: 9 - protein, 447 aa, synthetic construct
EVQLVQSGAEVKKPGESLKISCKGSGYSFTNYWIGWVRQMPGKGLEWMGIIYPGNSDTRFSPSFQGQVTISADKSITTAYLQWSSLKASDTAMYYCARHGTSSDYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDDNFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SYDMY

SEQ ID NO: 12 - protein, 16 aa, synthetic construct
GIDTVGDTYYPDSVKG

SEQ ID NO: 13 - protein, 13 aa, synthetic construct
GIYGDFYYYGLDV

SEQ ID NO: 14 - protein, 16 aa, synthetic construct
RSSQSLLYSDGNNYLD

SEQ ID NO: 15 - protein, 7 aa, synthetic construct
LGSNRAS

SEQ ID NO: 16 - protein, 8 aa, synthetic construct
MQALQTLT

SEQ ID NO: 17 - protein, 120 aa, synthetic construct
EVQLVESGGGWVQPGGSLRLSCAASGFTFSSYDMYWVRQATGKGLEWVSGIDTVGDTYYPDSVKGRFTISRENAKNSVYLQMNTLRAGDTAVYYCVRGIYGDFYYYGLDVWGHGTTVTVS

SEQ ID NO: 18 - protein, 112 aa, synthetic construct
DIVMTQSPLSLPVTPGEPASISCRSSQSLLYSDGNNYLDWYLQKPGQSPHLLIYLGSNRASGVPDRFSGSGSGTDFTLKISRVEADDVGVYYCMQALQTLTFGGGTKVEIKR

SEQ ID NO: 19 - protein, 447 aa, synthetic construct
EVQLVESGGGWVQPGGSLRLSCAASGFTFSSYDMYWVRQATGKGLEWVSGIDTVGDTYYPDSVKGRFTISRENAKNSVYLQMNTLRAGDTAVYYCVRGIYGDFYYYGLDVWGHGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 218 aa, synthetic construct
DIVMTQSPLSLPVTPGEPASISCRSSQSLLYSDGNNYLDWYLQKPGQSPHLLIYLGSNRASGVPDRFSGSGSGTDFTLKISRVEADDVGVYYCMQALQTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
TYDMH

SEQ ID NO: 22 - protein, 16 aa, synthetic construct
AIDLAGDTYYPGSVKG

SEQ ID NO: 23 - protein, 14 aa, synthetic construct
GGDGYNYDYYGIDV

SEQ ID NO: 24 - protein, 16 aa, synthetic construct
RSSQSLLHSDGYHYLD

SEQ ID NO: 25 - protein, 7 aa, synthetic construct
LGSNRAS

SEQ ID NO: 26 - protein, 8 aa, synthetic construct
MQALQTLT

SEQ ID NO: 27 - protein, 122 aa, synthetic construct
EVQLVESGGGLVQPGGSLRLSCAASGFTFSTYDMHWVRQTTGKGLEWVSAIDLAGDTYYPGSVKGRFTISREDAKNSLYLQMNSLRAGDTAVYYCARGGDGYNYDYYGIDVWGQGTTVTVSS

SEQ ID NO: 28 - protein, 112 aa, synthetic construct
DIVMTQSPLSLPVTPGEPASISCRSSQSLLHSDGYHYLDWYLQKPGQSPQLLIYLGSNRASGVPDRFSGSGSGTDFTLNISRVEAEDVGVYYCMQALQTLTFGGGTKVEIKR

SEQ ID NO: 29 - protein, 448 aa, synthetic construct
EVQLVESGGGLVQPGGSLRLSCAASGFTFSTYDMHWVRQTTGKGLEWVSAIDLAGDTYYPGSVKGRFTISREDAKNSLYLQMNSLRAGDTAVYYCARGGDGYNYDYYGIDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 218 aa, synthetic construct
DIVMTQSPLSLPVTPGEPASISCRSSQSLLHSDGYHYLDWYLQKPGQSPQLLIYLGSNRASGVPDRFSGSGSGTDFTLNISRVEAEDVGVYYCMQALQTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 17 aa, synthetic construct
IIYPGNSDTRFSPSFQG

SEQ ID NO: 32 - protein, 446 aa, synthetic construct
EVQLVQSGAEVKKPGESLKISCKGSGYSFTNYWIGWVRQMPGKGLEWMGIIYPGNSDTRFSPSFQGQVTISADKSITTAYLQWSSLKASDTAMYYCARHGTSSDYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 33 - protein, 446 aa, synthetic construct
EVQLVESGGGWVQPGGSLRLSCAASGFTFSSYDMYWVRQATGKGLEWVSGIDTVGDTYYPDSVKGRFTISRENAKNSVYLQMNTLRAGDTAVYYCVRGIYGDFYYYGLDVWGHGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 34 - protein, 447 aa, synthetic construct
EVQLVESGGGLVQPGGSLRLSCAASGFTFSTYDMHWVRQTTGKGLEWVSAIDLAGDTYYPGSVKGRFTISREDAKNSLYLQMNSLRAGDTAVYYCARGGDGYNYDYYGIDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 35 - protein, 8 aa, synthetic construct
GYSFTNYW

SEQ ID NO: 36 - protein, 6 aa, synthetic construct
IYPGNS

SEQ ID NO: 37 - protein, 16 aa, synthetic construct
YCARHGTSSDYYGLDV

SEQ ID NO: 38 - protein, 6 aa, synthetic construct
QDISNY

SEQ ID NO: 39 - skipped

SEQ ID NO: 40 - protein, 11 aa, synthetic construct
YCQQDDNFPLT

SEQ ID NO: 41 - DNA, 51 bp, synthetic construct
  source 1..51: mol_type = other DNA
agaaaggcctctagtttgactccctyggctgcccagaagcaatagtgcctg